(19) United States
(12) Patent Application Publication
(10) Pub. No.: US 2020/0002323 A1
(43) Pub. Date: Jan. 2, 2020

(54) COMPOUNDS

(71) Applicant: GLAXOSMITHKLINE INTELLECTUAL PROPERTY DEVELOPMENT LIMITED, Brentford (GB)

(72) Inventors: Haifeng CUI, Collegeville, PA (US); Feng REN, Shanghai (CN); Yingxia SANG, Shanghai (CN); Xiaomin ZHANG, Shanghai (CN)

(21) Appl. No.: 16/480,995

(22) PCT Filed: Jan. 23, 2018
(86) PCT No.: PCT/CN2018/073782

§ 371 Date: Jul. 25, 2019

(30) Foreign Application Priority Data
Jan. 25, 2017 (CN) PCT/CN2017/072586

(51) Int. Cl. C07D 413/14; C07D 401/14; A61P 25/16

(52) U.S. Cl. CPC C07D 413/14; A61P 25/16; C07D 401/14

(57) ABSTRACT

Provided are novel compounds that inhibit LRRK2 kinase activity, processes for their preparation, compositions containing them and their use in the treatment of or prevention of diseases associated with or characterized by LRRK2 kinase activity, for example Parkinson's disease, Alzheimer's disease and amyotrophic lateral sclerosis (ALS).

### FIELD OF THE INVENTION

The present invention relates to novel compounds that inhibit LRRK2 kinase activity, processes for their preparation, compositions containing them and their use in the treatment of diseases associated with or characterized by LRRK2 kinase activity, for example, Parkinson's disease, Alzheimer's disease and amyotrophic lateral sclerosis (ALS).

### BACKGROUND OF THE INVENTION

Parkinson's disease (PD) is a neurodegenerative disorder characterized by selective degeneration and cell death of dopaminergic neurons in the substantial nigra region of the brain. Parkinson's disease was generally considered to be sporadic and of unknown etiology, but, in the last 15 years, there has been an important development of the understanding of the genetic basis of this disease and associated pathogenic mechanisms. One area of the development is the understanding of leucine rich repeat kinase 2 (LRRK2) protein. A number of mis-sense mutations in the LRRK2 gene have been strongly linked with autosomal dominant Parkinson's disease in familial studies (See WO2006068492 and WO2006045392; Trinh and Farrer 2013, Nature Reviews in Neurology 9: 445-454; Paisan-Ruiz et al., 2013, J. Parkinson's Disease 3: 85-103). The G2019S mutation in LRRK2 is the most frequent mis-sense mutation and is associated with a clinical phenotype that closely resembles sporadic Parkinson's disease. The LRRK2 G2019S mutation is also present in approximately 1.5% of sporadic Parkinson's disease cases (See Gilks et al., 2005, Lancet, 365: 415-416). In addition to the known pathogenic coding mutations in LRRK2, additional amino acid coding variants of LRRK2 have been identified that are also associated with risk of developing Parkinson's disease (See Ross et al., 2011 Lancet Neurology 10: 898-908). Furthermore, genome-wide association studies (GWAS) have identified LRRK2 as a Parkinson's disease susceptibility locus, which indicates that LRRK2 may be also relevant to sporadic Parkinson's disease cases without mutations that cause amino acid substitutions in the LRRK2 protein. (See Satake et al., 2009 Nature Genetics 41:1303-1307; Simon-Sanchez et al 2009 Nature Genetics 41: 1308-1312)

LRRK2 is a member of the ROCO protein family and all members of this family share five conserved domains. The most common pathogenic mutation G2019S occurs in the highly conserved kinase domain of LRRK2. This mutation confers an increase in the LRRK2 kinase activity in in vitro enzyme assays of recombinant LRRK2 proteins (See Jaleel et al., 2007, Biochem J, 405: 307-317) and in LRRK2 proteins purified from G2019S PD patient-derived cells (See Dzamko et al., 2010 Biochem. J. 430: 405-413). A less frequent LRRK2 pathogenic mutation that confers amino acid substitution at a different residue, R1441, has also been shown to elevate LRRK2 kinase activity by decreasing the rate of GTP hydrolysis by the GTPase domain of LRRK2 (See Guo et al., 2007 Exp Cell Res. 313: 3658-3670; West et al., 2007 Hum. Mol Gen. 16: 223-232). Moreover, phosphorylation of Rab protein physiologic substrates of LRRK2 has been shown to be increased by a range of Parkinson's disease pathogenic mutations of LRRK2 (See Steger et al., 2016 eLife 5 e12813). Therefore, the evidence indicates that the kinase and GTPase activities of LRRK2 are important for pathogenesis, and that the LRRK2 kinase domain may regulate overall LRRK2 function (See Cookson, 2010 Nat. Rev. Neurosci. 11: 791-797).

There is evidence to show that the increased LRRK2 kinase activity is associated with neuronal toxicity in cell culture models (See Smith et al., 2006 Nature Neuroscience 9: 1231-1233) and kinase inhibitor compounds protect against LRRK2-mediated cell death (See Lee et al., 2010 Nat. Med. 16: 998-1000). LRRK2 has also been reported to act as a negative regulator of microglial-mediated clearance of alpha-synuclein (See Maekawa et al., 2016 BMC Neuroscience 17:77), suggesting a possible utility of LRRK2 inhibitors in promoting clearance of neurotoxic forms of alpha-synuclein in the treatment of Parkinson's disease.

Induced pluripotent stem cells (iPSCs) derived from LRRK2 G2019S Parkinson's disease patients have been found to exhibit defects in neurite outgrowth and increased susceptibility to rotenone, that may be ameliorated by either genetic correction of the G2019S mutation or treatment of cells with small molecule inhibitors of LRRK2 kinase activity (See Reinhardt et al., 2013 Cell Stem Cell 12: 354-367). Mitochondrial DNA damage has been reported as a molecular marker of vulnerable dopamine neurons in substantia nigra of postmortem Parkinson's disease specimens (See Sanders et al 2014 Neurobiol. Dis. 70: 214-223). Increased levels of such mitochondrial DNA damage associated with LRRK2 G2019S mutation in iSPCs is blocked by genetic correction of the G2019S mutation (See Sanders et al., 2014 Neurobiol. Dis. 62: 381-386).

Additional evidence links LRRK2 function and dysfunction with autophagy-lysosomal pathways (See Manzoni and Lewis, 2013 Faseb J. 27:3234-3429). LRRK2 proteins confer defects in chaperone-mediated autophagy that negatively impact the ability of cells to degrade alpha-synuclein (Orenstein et al., 2013 Nature Neurosci. 16 394-406). In other cell models, selective LRRK2 inhibitors have been shown to stimulate macroautophagy (See Manzoni et al., 2013 BBA Mol. Cell Res. 1833: 2900-2910). These data suggest that small molecule inhibitors of LRRK2 kinase activity may have utility in the treatment of diseases characterized by defects in cellular proteostasis that result from aberrant autophagy/lysosomal degradation pathways including forms of Parkinson's disease associated with GBA mutations (See Swan and Saunders-Pullman 2013 Curr. Neurol. Neurosci Rep. 13: 368), other alpha-synucleinopathies, tauopathies, Alzheimer's disease (See Li et al., 2010 Neurodegen. Dis. 7: 265-271) and other neurodegenerative diseases (See Nixon 2013 Nat. Med. 19: 983-997) and Gaucher disease (See Westbroek et al., 2011 Trends. Mol. Med. 17: 485-493). As promoters of autophagy, small molecule inhibitors of LRRK2 kinase may also have utility in treatment of other diseases including diabetes, obesity, motor neuron disease, epilepsy and some cancers (See Rubinsztein et al., 2012 Nat. Rev. Drug Discovery 11: 709-730), pulmonary diseases such as chronic obstructive pulmonary disease and idiopathic pulmonary fibrosis (See Araya et al., 2013 Intern. Med. 52: 2295-2303) and autoimmune diseases such as systemic lupus erythematosus (See Martinez et al., 2016 Nature 533: 115-119). As promoters of autophagy and phagocytic processes, small molecule inhibitors of LRRK2 kinase may also have utility in augmenting host responses in treatment of a range of intracellular bacterial infections, parasitic infections and viral infections, including diseases such as tuberculosis (See Rubinsztein et al., 2012 Nat. Rev. Drug Discovery 11: 709-730; Araya et al., 2013 Intern. Med. 52: 2295-2303; Gutierrez, Biochemical Society Conference; Leucine rich repeat kinase 2: ten years along the road to therapeutic intervention, Henley Business School, UK 12 Jul. 2016), HIV, West Nile Virus and chikungunya virus (see Shoji-Kawata et al., 2013 Nature 494: 201-206). LRRK2 inhibitors may have utility in treatment of such diseases alone, or in combination with drugs that directly target the infectious agent. Further, significantly elevated levels of LRRK2 mRNA have also been observed in fibroblasts of Niemann-Pick Type C (NPC) disease patients compared with fibroblasts of normal subjects, which indicates that aberrant LRRK2 function may play a role in lysosomal disorders (See Reddy et al., 2006 PLOS One 1 (1):e19 doi: 10.1371/journal.pone.0000019—supporting information Dataset 51). This observation suggests that LRRK2 inhibitors may have utility for treatment of NPC.

The PD-associated G2019S mutant form of LRRK2 has also been reported to enhance phosphorylation of tubulin-associated Tau (See Kawakami et al., 2012 PLoS ONE 7: e30834, doi 10.1371), and disease models have been proposed in which LRRK2 acts upstream of the pathogenic effects of Tau and alpha-synuclein (See Taymans & Cookson, 2010, BioEssays 32: 227-235). In support of this, LRRK2 expression has been associated with increased aggregation of insoluble Tau, and increased Tau phosphorylation, in a transgenic mouse model (See Bailey et al., 2013 Acta Neuropath. 126:809-827). Over-expression of the PD pathogenic mutant protein LRRK2 R1441G is reported to cause symptoms of Parkinson's disease and hyperphosphorylation of Tau in transgenic mouse models (See Li, Y. et al. 2009, Nature Neuroscience 12: 826-828). Therefore, these data suggest that LRRK2 inhibitors of kinase catalytic activity may be useful for the treatment of tauopathy diseases characterized by hyperphosphorylation of Tau such as argyrophilic grain disease, Pick's disease, corticobasal degeneration, progressive supranuclear palsy and inherited frontotemporal dementia and parkinsonism linked to chromosome 17 (FTDP-17) (See Goedert, M and Jakes, R (2005) Biochemica et Biophysica Acta 1739, 240-250). In addition, LRRK2 inhibitors may have utility in treatment of other diseases characterized by diminished dopamine levels such as withdrawal symptoms/relapse associated with drug addiction (See Rothman et al., 2008, Prog. Brain Res, 172: 385).

Other studies have also shown that overexpression of the G2019S mutant form of LRRK2 confers defects in subventricular zone (SVZ) neuroprogenitor cell proliferation and migration in transgenic mouse models (See Winner et al., 2011 Neurobiol. Dis. 41: 706-716) and reduces neurite length and branching cell culture models (See Dachsel et al., 2010 Parkinsonism & Related Disorders 16: 650-655). Moreover, it was reported that agents that promote SVZ neuroprogenitor cell proliferation and migration also improve neurological outcomes following ischemic injury in rodent models of stroke (See Zhang et al., 2010 J. Neurosci. Res. 88: 3275-3281). These findings suggest that compounds that inhibit aberrant activity of LRRK2 may have utility for the treatments designed to stimulate restoration of CNS functions following neuronal injury, such as ischemic stroke, traumatic brain injury, spinal cord injury.

Mutations in LRRK2 have also been identified that are clinically associated with the transition from mild cognitive impairment (MCI) to Alzheimer's disease (See WO2007149798). These data suggest that inhibitors of LRRK2 kinase activity may be useful for the treatment diseases such as Alzheimer's disease, other dementias and related neurodegenerative disorders.

Aberrant regulation of normal LRRK2 proteins is also observed in some disease tissues and models of disease. Normal mechanisms of translational control of LRRK2 by miR-205 are perturbed in some sporadic PD cases, where significant decreases in miR-205 levels in PD brain samples concur with elevated LRRK2 protein levels in those samples (See Cho et al., (2013) Hum. Mol. Gen. 22: 608-620). Therefore, LRRK2 inhibitors may be used in treatment of sporadic PD patients who have elevated levels of normal LRRK2 proteins.

In an experimental model of Parkinson's disease in marmosets, an elevation of LRRK2 mRNA is observed in a manner that correlates with the level of L-Dopa induced dyskinesia (See Hurley, M. J et al., 2007 Eur. J. Neurosci. 26: 171-177). This suggests that LRRK2 inhibitors may have a utility in amelioration of such dyskinesias.

Significantly elevated levels of LRRK2 mRNA have been reported in ALS patient muscle biopsy samples (See Shtilbans et al., 2011 Amyotrophic Lateral Sclerosis 12: 250-256) It is suggested that elevated levels of LRRK2 kinase activity may be a characteristic feature of ALS. Therefore, this observation indicated that LRRK2 inhibitor may have utility for treatment of ALS.

There is also evidence indicating that LRRK2 kinase activity may play a role in mediating microglial proinflammatory responses (See Moehle et al., 2012, J. Neuroscience 32: 1602-1611). This observation suggests a possible utility of LRRK2 inhibitors for treatment of aberrant neuroinflammatory mechanisms that contribute a range of neurodegenerative diseases, including Parkinson's disease, Alzheimer's disease, multiple sclerosis, HIV-induced dementia, amyotrophic lateral sclerosis, ischemic stroke, traumatic brain injury and spinal cord injury. Some evidence also indicates that LRRK2 plays a role in regulating neuronal progenitor differentiation in vitro (See Milosevic, J. et al., 2009 Mol. Neurodegen. 4: 25). This evidence suggests that inhibitors of LRRK2 may have a utility in production of neuronal progenitor cells in vitro for consequent therapeutic application in cell based-treatment of CNS disorders.

It has been reported that Parkinson's disease patients bearing LRRK2 G2019S mutation display increased frequency of non-skin cancers, including renal, breast, lung, prostate cancers as well as acute myelogenous leukemia (AML). Since there is evidence to show that G2019S mutation in LRRK2 increases catalytic activity of the LRRK2 kinase domain, small molecule inhibitors of LRRK2 may have a utility in treatment of cancers, for example kidney cancer, breast cancer, lung cancer, prostate cancer (e.g. solid tumors) and blood cancer (See. AML; Saunders-Pullman et al., 2010, Movement Disorders, 25:2536-2541; Inzelberg et al., 2012 Neurology 78: 781-786). Amplification and over-expression of LRRK2 has also been reported in papillary renal and thyroid carcinomas, where co-operativity between LRRK2 and the MET oncogene may promote tumor cell growth and survival (See Looyenga et al., 2011 PNAS 108: 1439-1444.)

Some studies suggested that genetic association of common LRRK2 variants with susceptibility to ankylosing spondylitis (See Danoy P, et al., 2010. PLoS Genet.; 6(12):e1001195; and leprosy infection. (See Zhang F R, et al. 2009, N Engl J Med. 361:2609-18.) These findings suggest that inhibitors of LRRK2 may have a utility in the treatment of ankylosing spondylitis and leprosy infection.

Meta-analysis of three genome wide associated scans for Crohn's disease identified a number of loci associated with the disease, including the locus containing the LRRK2 gene (See Barrett et al., 2008, Nature Genetics, 40: 955-962). Evidence has also emerged that LRRK2 is an IFN-γ target gene that may be involved in signaling pathways relevant to Crohn's disease pathogenesis (See Gardet et al., 2010, J. Immunology, 185: 5577-5585). These findings suggest that inhibitors of LRRK2 may have utility in the treatment of Crohn's disease.

As an IFN-γ target gene, LRRK2 may also play a role in T cell mechanisms that underlie other diseases of the immune system such as multiple sclerosis and rheumatoid arthritis. Further potential utility of LRRK2 inhibitors comes from the reported finding that B lymphocytes constitute a major population of LRRK2 expressing cells (See Maekawa et al. 2010, BBRC 392: 431-435). This suggests that LRRK2 inhibitors may be effective in treatment of diseases of the immune system for which B cell depletion is, or may be, effective in diseases such as lymphomas, leukemias, multiple sclerosis (See Ray et al., 2011 J. Immunol. 230: 109), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, Sjogren's syndrome, Devic's disease and inflammatory myopathies (See Engel et al., 2011 Pharmacol. Rev. 63: 127-156; Homam et al., 2010 J. Clin. Neuromuscular Disease 12: 91-102).

WO2016036586 and WO2017012576 disclose a series of compounds described as inhibitors of LRRK2 kinase and their use in the treatment of diseases, including, inter alia, Parkinson's disease. Unmet needs exist for new treatments that will halt or slow disease progression both in terms of motor (e.g. control of gait dysfunction, freezing, and postural imbalance) and non-motor symptoms (e.g. PD-associated dementia), reducing the need for escalating use of symptomatic medications and associated long-term adverse effects of currently available treatment (e.g. dyskinesia and on/off fluctuations) maintaining independence for longer.

### SUMMARY OF THE INVENTION

The present invention provides, in a first aspect, compounds of Formula (I) and pharmaceutically acceptable salts thereof:

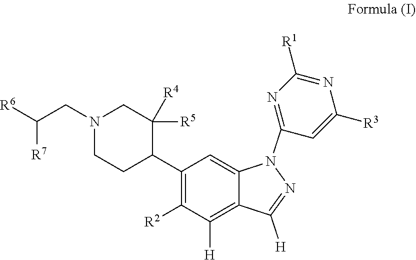

wherein
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃haloalkyl;
R³ is selected from the group consisting of:
a) an N-linked 4-6 membered heterocyclyl ring optionally substituted with one or two substituents independently selected from the group consisting of:
   halo,
   hydroxyl,
   C₁₋₆alkyl, which alkyl group is optionally substituted with one or two substituents independently selected from the group consisting of: halo, hydroxyl, C₁₋₃alkoxy and cyclopropyl, and
   C₁₋₆ alkoxyl, which alkoxyl group is optionally substituted with one or two substitutents independently selected from the group consisting of halo, hydroxyl and C₁₋₃ alkoxyl,
   wherein when the N-linked 4-6 membered heterocyclyl ring contains a substitutable nitrogen atom, the group of substitutents also includes a 4-6 membered heterocyclyl ring which is optionally substituted with one, two or three substitutents independently selected from halo, hydroxyl, and C₁₋₃ alkoxyl, with the proviso that the 4-6 membered heterocyclyl ring is attached to said substitutable nitrogen atom;
b) NHR⁸; and
c) OR⁸;
R⁴ and R⁵ are independently selected from the group consisting of H, hydroxyl and halo;
R⁶ is halo, hydroxyl or —(CH₂)ₙSO₂C₁₋₃alkyl, wherein n is 0, 1, 2 or 3;
R⁷ is selected from the group consisting of
   H,
   Cyclopropyl,
   C₁₋₃alkyl, optionally substituted with one, two or three substitutents independently selected from the group consisting of halo, hydroxyl, and C₁₋₃ alkoxyl,
   —CH₂CH₂— and —CH₂CH₂CH₂—, wherein one terminal carbon joins with the carbon atom to which another terminal carbon atom is attached to form a ring;
R⁸ is independently selected from the group consisting of:
   C₄₋₆ cycloalkyl, optionally substituted with one, two or three substituents independently selected from the group consisting of
      halo,
      hydroxyl,
      C₁₋₃alkoxyl and
      C₁₋₃ alkyl, optionally substituted with one two or three substituents independently selected from halo and hydroxyl; and
   a 4-6 membered heterocyclyl that contains nitrogen or oxygen and is optionally substituted with one or more substitutents independently selected from the group consisting of
      halo,
      hydroxyl,
      C₁₋₃alkoxyl and
      C₁₋₃ alkyl, optionally substituted with one two or three substituents independently selected from halo or hydroxyl.

In a further aspect of the invention, the invention provides a pharmaceutical composition comprising a compound of Formula (I) or a pharmaceutically acceptable salt thereof and a pharmaceutically acceptable carrier.

A further aspect of the invention provides a compound of Formula (I) or a pharmaceutically acceptable salt thereof for use in the treatment or prevention of Parkinson's disease, Alzheimer's disease, or amyotrophic lateral sclerosis (ALS).

### DETAILED DESCRIPTION OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will Fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Generally, the nomenclature used herein and the laboratory procedures in organic chemistry, medicinal chemistry, biology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

### A. Definitions

As used herein, “alkyl” refers to a monovalent, saturated hydrocarbon chain having a specified number of carbon atoms. For example, C₁₋₃ alkyl refers to an alkyl group having from 1 to 3 carbon atoms. Alkyl groups may be straight or branched. In some embodiments, branched alkyl groups may have one, two, or three branches. Exemplary alkyl groups include, but are not limited to, methyl, ethyl, and propyl (n-propyl and isopropyl).

As used herein, “alkoxy” refers to the group —O-alkyl. For example, C₁₋₆ alkoxy groups contain from 1 to 6 carbon atoms. C₁₋₃ alkoxy groups contain from 1 to 3 carbon atoms.

Exemplary alkoxy groups include, but are not limited to, methoxy, ethoxy, propoxy, butoxyl, pentyloxy, and hexyloxy.

As used herein, “cycloalkyl” refers to a saturated monocyclic hydrocarbon ring having a specified number of carbon atoms. For example, C₃₋₆ cycloalkyl contains 3 to 6 carbon atoms as member atoms in the ring. Examples of C₃₋₆ cycloalkyl include cyclopropyl, cyclobutyl, cyclopentyl and cyclohexyl.

As used herein, “halogen” refers to fluorine (F), chlorine (Cl), bromine (Br), or iodine (I). “Halo” refers to the halogen radicals: fluoro (—F), chloro (—Cl), bromo (—Br), or iodo (—I).

As used herein, “haloalkyl” refers to an alkyl group, as defined above, having one or more halogen atoms selected from F, Cl, Br, or I, which are substituted on any or all of the carbon atoms of the alkyl group by replacing hydrogen atoms attached to the carbon atoms and which may be the same or different. For example, C₁₋₃haloalkyl refers to a C₁₋₃alkyl group substituted with one or more halogen atoms. In some embodiments, “haloalkyl” refers to an alkyl group substituted with one or more halogen atoms independently selected from F or Cl. Exemplary haloalkyl groups include, but are not limited to, chloromethyl, bromoethyl, trifluoromethyl, and dichloromethyl.

As used herein, “heterocyclyl” or “herterocyclyl ring” is a monovalent radical derived by removal of a hydrogen atom from a saturated monocyclic ring, which ring consists of ring carbon atoms and 1 or more ring heteroatoms independently selected from nitrogen, oxygen or sulphur. In one embodiment, the ring consists of ring carbon atoms and 1 to 3 ring heteroatoms independently selected from nitrogen, oxygen or sulphur. In one embodiment, the ring-heteroatoms are independently selected from nitrogen or oxygen. The number of ring atoms may be specified. For example, a “4-6 membered heterocyclyl” a heterocyclyl as defined above consisting of 4-6 ring atoms. The term N-linked 4-6 membered heterocyclyl ring refers to a 4-6 membered heterocyclyl ring as defined above that contains at least one nitrogen ring atom through which it is linked to the core. Other ring heteroatoms (nitrogen, oxygen or sulphur) may additionally be present. The term nitrogen containing heterocyclyl refers to heterocyclyl ring as defined above that contains at least one nitrogen ring atom. Other ring heteroatoms (nitrogen, oxygen or sulphur) may additionally be present. The term oxygen containing heterocyclyl should be construed in an analogous manner. Examples of herterocyclyl rings include, but are not limited to, azetidinyl, tetrahydrofuranyl (including, for example, tetrahydrofuran-2-yl and tetrahydrofuran-3-yl), pyrrolidinyl (including, for example, pyrrolidin-1-yl and pyrrolidin-3-yl), piperidinyl (including, for example, piperidin-3-yl and piperidin-4-y), morpholinyl (including, for example, morpholin-2-yl and morpholin-4-yl).

As used herein, “substituted” in reference to a group indicates that one or more hydrogen atom attached to a member atom (e.g., carbon atom) within the group is replaced with a substituent selected from the group of defined substituents. It should be understood that the term “substituted” includes the implicit provision that such substitution is in accordance with the permitted valence of the substituted atom and the substituent and that the substitution results in a stable compound (i.e. one that does not spontaneously undergo transformation such as by rearrangement, cyclization, or elimination and that is sufficiently robust to survive isolation from a reaction mixture). When it is stated that a group may contain one or more substituent, one or more (as appropriate) member atom within the group may be substituted. In addition, a single member atom within the group may be substituted with more than one substituent as long as such substitution is in accordance with the permitted valence of the atom. Examples of substituted heterocyclyl rings rings include, but are not limited to,

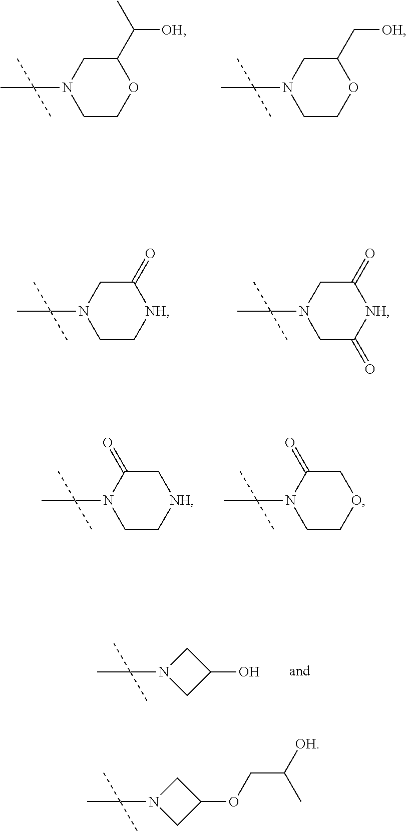

As used herein, “optionally substituted” indicates that a particular group may be unsubstituted, or may be substituted as further defined.

As used herein, the term “disease” refers to any alteration in state of the body or of some of the organs, interrupting or disturbing the performance of the functions and/or causing symptoms such as discomfort, dysfunction, distress, or even death to the person afflicted or those in contact with a person. A disease can also include a distemper, ailing, ailment, malady, disorder, sickness, illness, complain, interdisposition and/or affectation.

As used herein, “treat”, “treating” or “treatment” in reference to a disease means: (1) to ameliorate the disease or one or more of the biological manifestations of the disease, (2) to interfere with (a) one or more points in the biological cascade that leads to or is responsible for the disease or (b) one or more of the biological manifestations of the disease, (3) to alleviate one or more of the symptoms or effects associated with the disease, (4) to slow the progression of the disease or one or more of the biological manifestations of the disease, and/or (5) to diminish the likelihood of severity of a disease or biological manifestations of the disease. Symptomatic treatment refers to treatment as referred to in point (1), (3) and (5). Disease modifying treatment refers to treatment as defined in point (2) and (4).

As used herein, “prevent”, “preventing” or “prevention” means the prophylactic administration of a drug to diminish the likelihood of the onset of or to delay the onset of a disease or biological manifestation thereof.

As used herein, “subject” means a mammalian subject (e.g., dog, cat, horse, cow, sheep, goat, monkey, etc.), and human subjects including both male and female subjects, and including neonatal, infant, juvenile, adolescent, adult and geriatric subjects, and further including various races and ethnicities including, but not limited to, white, black, Asian, American Indian and Hispanic.

As used herein, “pharmaceutically acceptable salt(s)” refers to salt(s) that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

As used herein, “therapeutically effective amount” in reference to a compound of the invention or other pharmaceutically-active agent means an amount of the compound sufficient to treat or prevent the patient's disease but low enough to avoid serious side effects (at a reasonable benefit/risk ratio) within the scope of sound medical judgment. A therapeutically effective amount of a compound will vary with the particular compound chosen (e.g. consider the potency, efficacy, and half-life of the compound); the route of administration chosen; the disease being treated; the severity of the disease being treated; the age, size, weight, and physical disease of the patient being treated; the medical history of the patient to be treated; the duration of the treatment; the nature of concurrent therapy; the desired therapeutic effect; and like factors, but can nevertheless be routinely determined by the skilled artisan.

### B. Compounds

This invention provides, in a first aspect, a compound of Formula (I) and salts thereof:

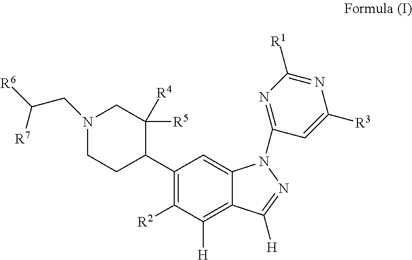

wherein
wherein
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃haloalkyl;
R³ is selected from the group consisting of:
d) an N-linked 4-6 membered heterocyclyl ring optionally substituted with one or two substituents independently selected from the group consisting of:
   halo,
   hydroxyl,
   C₁₋₆alkyl, which alkyl group is optionally substituted with one or two substituents independently selected from the group consisting of: halo, hydroxyl, C₁₋₃alkoxy and cyclopropyl, and
   C₁₋₆ alkoxyl, which alkoxyl group is optionally substituted with one or two substitutents independently selected from the group consisting of halo, hydroxyl and C₁₋₃ alkoxyl,
   wherein when the N-linked 4-6 membered heterocyclyl ring contains a substitutable nitrogen atom, the group of substitutents also includes a 4-6 membered heterocyclyl ring which is optionally substituted with one, two or three substitutents independently selected from halo, hydroxyl, and C₁₋₃ alkoxyl, with the proviso that the 4-6 membered heterocyclyl ring is attached to said substitutable nitrogen atom;
e) NHR⁸; and
f) OR⁸;
R⁴ and R⁵ are independently selected from the group consisting of H, hydroxyl and halo;
R⁶ is halo, hydroxyl or —(CH₂)ₙSO₂C₁₋₃alkyl, wherein n is 0, 1, 2, or 3;
R⁷ is selected from the group consisting of
   H,
   Cyclopropyl,
   C₁₋₃alkyl, optionally substituted with one, two or three substitutents independently selected from the group consisting of halo, hydroxyl, and C₁₋₃ alkoxyl,
   —CH₂CH₂— and —CH₂CH₂CH₂—, wherein one terminal carbon joins with the carbon atom to which another terminal carbon atom is attached to form a ring;
R⁸ is independently selected from the group consisting of:
   C₄₋₆ cycloalkyl, optionally substituted with one, two or three substituents independently selected from the group consisting of
      halo,
      hydroxyl,
      C₁₋₃ alkoxyl and
      C₁₋₃ alkyl, optionally substituted with one two or three substituents independently selected from halo and hydroxyl; and
   a 4-6 membered heterocyclyl that contains nitrogen or oxygen and is optionally substituted with one or more substitutents independently selected from the group consisting of
      halo,
      hydroxyl,
      C₁₋₃ alkoxyl and
      C₁₋₃ alkyl, optionally substituted with one two or three substituents independently selected from halo or hydroxyl.

In one embodiment, R¹ is selected from the group consisting of C₁₋₃ alkyl and C₁₋₃ alkoxyl. In one embodiment, R¹ is selected from the group consisting of methyl or methoxy. In one embodiment, R¹ is methyl.

In one embodiment, R² is selected from the group consisting of H, halo and C₁₋₃alkyl. In one embodiment, R² is C₁₋₃alkyl. In one embodiment, R² is selected from the group consisting of H, halo and methyl. In one embodiment, R² is selected from the group consisting of H, fluoro, chloro and methyl. In one embodiment, R² is selected from the group consisting of H, chloro and methyl. In one embodiment, R² is selected from the group consisting of chloro and methyl. In one embodiment, R² is methyl.

In one embodiment R³ is an N-linked 4-6 membered heterocyclyl ring optionally substituted with one or two substituents independently selected from the group consisting of:
halo,
hydroxyl,
C₁₋₆alkyl, which alkyl group is optionally substituted with one or two substituents independently selected from the group consisting of: halo, hydroxyl, C₁₋₃alkoxy and cyclopropyl,
C₁₋₆ alkoxyl, which alkoxyl group is optionally substituted with one or two substitutents independently selected from halo, hydroxyl and C₁₋₃ alkoxyl, and where the N-linked 4-6 membered heterocyclyl ring contains a substitutable nitrogen atom, a further 4-6 membered heterocyclyl ring which is optionally substituted with one, two or three substitutents independently selected from halo, hydroxyl, and C₁₋₃ alkoxyl, and with the proviso that the further 4-6 membered heterocyclyl ring is attached to said substitutable nitrogen atom.

In one embodiment R³ is an N-linked 4-6 membered heterocyclyl ring optionally substituted with one or two substituents independently selected from the group consisting of:
halo,
hydroxyl,
C₁₋₃alkyl, which alkyl group is optionally substituted with one or two substituents independently selected from the group consisting of: halo, hydroxyl and C₁₋₃alkoxy, and
C₁₋₃ alkoxyl, which alkoxyl group is optionally substituted with one or two substitutents independently selected from halo, hydroxyl and C₁₋₃ alkoxyl.

In one embodiment R³ is an N-linked 4-6 membered heterocyclyl ring selected from the group consisting of morpholinyl, azetidinyl, pyrrolidinyl and piperazinyl, optionally substituted with one or two substituents independently selected from the group consisting of:
halo,
hydroxyl,
C₁₋₃alkyl, which alkyl group is optionally substituted with one or two substituents independently selected from the group consisting of: halo, hydroxyl and C₁₋₃alkoxy, and
C₁₋₃ alkoxyl, which alkoxyl group is optionally substituted with one or two substitutents independently selected from halo, hydroxyl and C₁₋₃ alkoxyl.

In one embodiment R³ is an N-linked 4-6 membered heterocyclyl ring selected from the group consisting of morpholinyl, azetidinyl, pyrrolidinyl and piperazinyl, optionally substituted with one or two substituents independently selected from the group consisting of:
hydroxyl,
C₁₋₃alkyl, which alkyl group is optionally substituted with one or two substituents independently selected from the group consisting of: halo, hydroxyl and C₁₋₃alkoxy, and
C₁₋₃ alkoxyl, which alkoxyl group is optionally substituted with one or two substitutents independently selected from halo, hydroxyl and C₁₋₃ alkoxyl.

In one embodiment R³ is an N-linked morpholinyl ring optionally substituted with one or two substituents independently selected from the group consisting of:
hydroxyl,
C₁₋₃alkyl, which alkyl group is optionally substituted with one or two substituents independently selected from the group consisting of: halo, hydroxyl and C₁₋₃alkoxy, and
C₁₋₃ alkoxyl, which alkoxyl group is optionally substituted with one or two substitutents independently selected from halo, hydroxyl and C₁₋₃ alkoxyl.

In one embodiment R³ is an N-linked morpholinyl ring optionally substituted with one C₁₋₃alkyl substituent, which alkyl group is optionally substituted with one or two substituents independently selected from the group consisting of: halo, hydroxyl and C₁₋₃alkoxy.

In one embodiment, R³ is morpholin-4-yl.

In one embodiment, R³ is 3-methyl morpholin-4-yl.

In one embodiment, R³ is (2-hydroxyethyl)-morpholin-4-yl.

In one embodiment, R³ is 3-hydroxyl azetidin-1-yl

In one embodiment R³ is an N-linked 4-6 membered heterocyclyl ring containing a substitutable nitrogen atom, substituted with a further 4-6 membered heterocyclyl ring which is optionally substituted with one, two or three substitutents independently selected from halo, hydroxyl, and C₁₋₃alkoxyl, and with the proviso that the further 4-6 membered heterocyclyl ring is attached to said substitutable nitrogen atom.

In one embodiment R³ is an N-linked 4-6 membered heterocyclyl ring containing a substitutable nitrogen atom, substituted with an oxetanyl group on said substitutable nitrogen atom.

In one embodiment, R⁴ and R⁵ are independently selected from the group consisting of H and halo. In one embodiment, R⁴ and R⁵ are independently selected from the group consisting of H and fluoro. In one embodiment, R⁴ and R⁵ are both hydrogen. In one embodiment, R⁴ is H and R⁵ is fluoro.

In one embodiment, R⁶ is fluoro or hydroxyl.

In one embodiment, R⁶ is —SO₂CH₃.

In one embodiment, R⁷ is H.

In one embodiment, R⁶ is hydroxyl and R⁷ is methyl.

In one embodiment, the invention provides a compound of formula (I) or a pharmaceutically acceptable salt thereof that is a compound of any one of examples 1 to 43, or a pharmaceutically acceptable salt thereof.

In one embodiment, this invention relates to a compound selected from

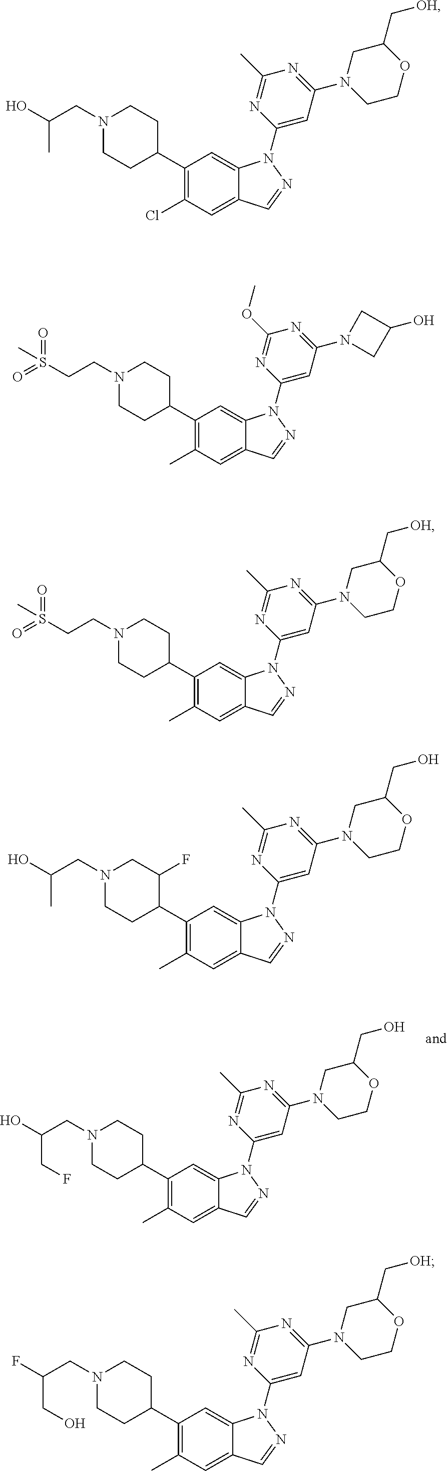 [SMILES: Cc1nc(N2CCOC(CO)C2)cc(-n2ncc3cc(Cl)c(C4CCN(CC(C)O)CC4)cc32)n1 ; COc1nc(N2CC(O)C2)cc(-n2ncc3cc(C)c(C4CCN(CCS(C)(=O)=O)CC4)cc32)n1 ; Cc1nc(N2CCOC(CO)C2)cc(-n2ncc3cc(C)c(C4CCN(CCS(C)(=O)=O)CC4)cc32)n1 ; Cc1nc(N2CCOC(CO)C2)cc(-n2ncc3cc(C)c(C4CCN(CC(C)O)CC4F)cc32)n1 ; Cc1nc(N2CCOC(CO)C2)cc(-n2ncc3cc(C)c(C4CCN(CC(O)CF)CC4)cc32)n1 ; Cc1nc(N2CCOC(CO)C2)cc(-n2ncc3cc(C)c(C4CCN(CC(F)CO)CC4)cc32)n1]

or a pharmaceutically acceptable salt thereof.

In one embodiment the invention provides a compound selected from
2-(3-fluoro-4-(1-(2-methoxy-6-morpholinopyrimidin-4-yl)-5-methyl-1H-indazol-6-yl)piperidin-1-yl)ethanol,
2-(4-(1-(6-(azetidin-1-yl)-2-methoxypyrimidin-4-yl)-5-methyl-1H-indazol-6-yl)-3-fluoropiperidin-1-yl)ethanol,
1-(4-(5-chloro-1-(6-(2-(hydroxymethyl)morpholino)-2-methylpyrimidin-4-yl)-1H-indazol-6-yl)piperidin-1-yl)propan-2-ol,
(4-(2-methyl-6-(5-methyl-6-(1-(2-(methylsulfonyl)ethyl)piperidin-4-yl)-1H-indazol-1-yl)pyrimidin-4-yl)morpholin-2-yl)methanol,
1-(3-fluoro-4-(1-(6-(2-(hydroxymethyl)morpholino)-2-methylpyrimidin-4-yl)-5-methyl-1H-indazol-6-yl)piperidin-1-yl)propan-2-ol,
1-fluoro-3-(4-(1-(6-(2-(hydroxymethyl)morpholino)-2-methylpyrimidin-4-yl)-5-methyl-1H-indazol-6-yl)piperidin-1-yl)propan-2-ol, and
2-fluoro-3-(4-(1-(6-(2-(hydroxymethyl)morpholino)-2-methylpyrimidin-4-yl)-5-methyl-1H-indazol-6-yl)piperidin-1-yl)propan-1-ol;
   or a pharmaceutically acceptable salt thereof.

In addition to the free base form of the compounds described herein, the salt form of the compounds is also within the scope of the present invention. The salts or pharmaceutically-acceptable salts of the compounds described herein may be prepared in situ during the final isolation and purification of the compound, or by separately reacting the purified compound in its free base form with a suitable base or acid, respectively. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

Certain compounds of formula (I) contain a basic group and are therefore capable of forming pharmaceutically-acceptable acid addition salts by treatment with a suitable acid. Suitable acids include pharmaceutically-acceptable inorganic acids and pharmaceutically-acceptable organic acids. Exemplary pharmaceutically-acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate. In some embodiments, the pharmaceutically acceptable salts include the L-tartrate, ethanedisulfonate (edisylate), sulfate, phosphate, p-toluenesulfonate (tosylate), hydrochloride salt, methanesulfonate, citrate, fumarate, benzenesulfonate, maleate, hydrobromate, L-lactate, malonate, and S-camphor-10-sulfonate. In certain embodiments, some of these salts form solvates. In certain embodiments, some of these salts are crystalline.

Certain compounds of Formula (I) or salts thereof may exist in stereoisomeric forms (e.g., they may contain one or more asymmetric carbon atoms). The individual stereoisomers (enantiomers and diastereomers) and mixtures of these are included within the scope of the present invention. The different isomeric forms may be separated or resolved one from the other by conventional methods, or any given isomer may be obtained by conventional synthetic methods or by stereospecific or asymmetric syntheses.

Certain compounds of Formula (I) are capable of existing in tautomeric forms. For example, certain compounds exhibit keto-enol tautomerism. In some cases, only one of a pair of tautomeric forms fall within Formula (I). Such alternative tautomers also form part of the invention.

The invention also includes isotopically-labelled compounds and salts, which are identical to compounds of Formula (I) or salts thereof, but for the fact that one or more atoms are replaced by an atom having an atomic mass or mass number different from the atomic mass or mass number most commonly found in nature. Examples of isotopes that can be incorporated into compounds of Formula (I) or salts thereof isotopes of hydrogen, carbon, nitrogen, fluorine, such as ³H, ¹¹C, ¹⁴C and ¹⁸F. Such isotopically-labelled compound of Formula (I) or salts thereof are useful in drug and/or substrate tissue distribution assays. For example, ¹¹C and ¹⁸F isotopes are useful in PET (positron emission tomography). PET is useful in brain imaging. Isotopically-labelled compounds of Formula (I) and salts thereof can generally be prepared by carrying out the procedures disclosed below, by substituting a readily available isotopically-labelled reagent for a non-isotopically labelled reagent. In one embodiment, compounds of Formula (I) or salts thereof are not isotopically labelled.

Certain compounds of Formula (I) or salts thereof may exist in solid or liquid form. In the solid state, compounds of Formula (I) or salts may exist in crystalline or noncrystalline form, or as a mixture thereof. For compounds of Formula (I) or salts that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water.

The skilled artisan will further appreciate that certain compounds of Formula (I), pharmaceutically acceptable salts or solvates thereof that exist in crystalline form, including the various solvates thereof, may exhibit polymorphism (i.e. the capacity to occur in different crystalline structures). These different crystalline forms are typically known as “polymorphs.” Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The skilled artisan also appreciates that this invention may contain various deuterated forms of compounds of Formula (I), or pharmaceutically acceptable salts thereof. Each available hydrogen atom attached to a carbon atom may be independently replaced with a deuterium atom. A person of ordinary skill in the art will know how to synthesize deuterated forms of compounds of Formula (I), or pharmaceutically acceptable salts thereof. Commercially available deuterated starting materials may be employed in the preparation of deuterated forms of compounds of Formula (I) or pharmaceutically acceptable salts thereof, or they may be synthesized using conventional techniques employing deuterated reagents (e.g. lithium aluminum deuteride).

### C. Methods of Use

Compounds of Formula (I) or pharmaceutically acceptable salts thereof are inhibitors of LRRK2 kinase activity and are thus believed to be of potential use in the treatment of or prevention of the following neurological diseases: Parkinson's disease, Alzheimer's disease, dementia (including Lewy body dementia and vascular dementia, HIV-induced dementia), amyotrophic lateral sclerosis (ALS), age related memory dysfunction, mild cognitive impairment, argyrophilic grain disease, Pick's disease, corticobasal degeneration, progressive supranuclear palsy, inherited frontotemporal dementia and parkinsonism linked to chromosome 17 (FTDP-17), withdrawal symptoms/relapse associated with drug addiction, L-Dopa induced dyskinesia, ischemic stroke, traumatic brain injury, spinal cord injury and multiple sclerosis. Other diseases potentially treatable by inhibition of LRRK2 include, but are not limited to, lysosomal disorders (for example, Niemann-Pick Type C disease, Gaucher disease), Crohn's disease, cancers (including thyroid, renal (including papillary renal), breast, lung and prostate cancers, leukemias (including acute myelogenous leukemia (AML)) and lymphomas), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, obesity, epilepsy, pulmonary diseases such as chronic obstructive pulmonary disease, idiopathic pulmonary fibrosis, Sjogren's syndrome, Devic's disease, inflammatory myopathies, ankylosing spondylitis, bacterial infections (including leprosy), viral infections (including tuberculosis, HIV, West Nile virus and chikungunya virus) and parasitic infections.

One aspect of the invention provides a compound of Formula (I) or a pharmaceutically acceptable salt thereof for use in therapy. In one embodiment, the invention provides a compound of Formula (I) or a pharmaceutically acceptable salt thereof for use in the treatment of or prevention of the above disorders (i.e. the neurological diseases and other diseases listed above). In one embodiment, the invention provides a compound of Formula (I) or a pharmaceutically acceptable salt thereof for use in the treatment of or prevention of Parkinson's disease. In one embodiment, the invention provides a compound of Formula (I) or a pharmaceutically acceptable salt thereof for use in the treatment of Parkinson's disease. In another embodiment, the invention provides a compound of Formula (I) or a pharmaceutically acceptable salt thereof for use in the treatment of or prevention of Alzheimer's disease. In one embodiment, the invention provides a compound of Formula (I) or a pharmaceutically acceptable salt thereof for use in the treatment of Alzheimer's disease. In another embodiment, the invention provides a compound of Formula (I) or a pharmaceutically acceptable salt thereof for use in the treatment of amyotrophic lateral sclerosis (ALS).

In one embodiment, the invention provides 1-(4-(5-chloro-1-(6-(2-(hydroxymethyl)morpholino)-2-methylpyrimidin-4-yl)-1H-indazol-6-yl)piperidin-1-yl)propan-2-ol or a pharmaceutically acceptable salt thereof for use in the treatment or prevention of Parkinson's disease, Alzheimer's disease or amyotrophic lateral sclerosis (ALS).

In another embodiment, the invention provides 1-(4-(5-chloro-1-(6-(2-(hydroxymethyl)morpholino)-2-methylpyrimidin-4-yl)-1H-indazol-6-yl)piperidin-1-yl)propan-2-ol or a pharmaceutically acceptable salt thereof for use in the treatment of Parkinson's disease.

In one embodiment, the invention provides (4-(2-methyl-6-(5-methyl-6-(1-(2-(methylsulfonyl)ethyl)piperidin-4-yl)-1H-indazol-1-yl)pyrimidin-4-yl)morpholin-2-yl)methanol or a pharmaceutically acceptable salt thereof for use in the treatment or prevention of Parkinson's disease, Alzheimer's disease or amyotrophic lateral sclerosis (ALS).

In another embodiment, the invention provides (4-(2-methyl-6-(5-methyl-6-(1-(2-(methylsulfonyl)ethyl)piperidin-4-yl)-1H-indazol-1-yl)pyrimidin-4-yl)morpholin-2-yl)methanol or a pharmaceutically acceptable salt thereof for use in the treatment of Parkinson's disease.

A further aspect of the invention provides the use of a compound of Formula (I) or a pharmaceutically acceptable salt thereof in the manufacture of a medicament for the treatment or prevention of the above disorders (i.e. the neurological diseases and other diseases listed above). A further aspect of the invention provides the use of a compound of Formula (I) or a pharmaceutically acceptable salt thereof in the manufacture of a medicament for the treatment of or prevention of Parkinson's disease. A further aspect of the invention provides the use of a compound of Formula (I) or a pharmaceutically acceptable salt thereof in the manufacture of a medicament for the treatment of Parkinson's disease. In another embodiment, the invention provides the use of a compound of Formula (I) or a pharmaceutically acceptable salt thereof in the manufacture of a medicament for the treatment or prevention of Alzheimer's disease. In one embodiment, the invention provides the use of a compound of Formula (I) or a pharmaceutically acceptable salt thereof in the manufacture of a medicament for the treatment of Alzheimer's disease. In another embodiment, the invention provides use of a compound of Formula (I) or a pharmaceutically acceptable salt thereof in the manufacture of a medicament for the treatment of amyotrophic lateral sclerosis (ALS).

In one embodiment, the invention provides the use of 1-(4-(5-chloro-1-(6-(2-(hydroxymethyl)morpholino)-2-methylpyrimidin-4-yl)-1H-indazol-6-yl)piperidin-1-yl)propan-2-ol or a pharmaceutically acceptable salt thereof in the manufacture of a medicament for the treatment or prevention of Parkinson's disease, Alzheimer's disease or amyotrophic lateral sclerosis (ALS).

In another embodiment, the invention provides the use of 1-(4-(5-chloro-1-(6-(2-(hydroxymethyl)morpholino)-2-methylpyrimidin-4-yl)-1H-indazol-6-yl)piperidin-1-yl)propan-2-ol or a pharmaceutically acceptable salt thereof in the manufacture of a medicament for the treatment or prevention of Parkinson's disease.

In yet another embodiment, the invention provides the use of 1-(4-(5-chloro-1-(6-(2-(hydroxymethyl)morpholino)-2-methylpyrimidin-4-yl)-1H-indazol-6-yl)piperidin-1-yl)propan-2-ol or a pharmaceutically acceptable salt thereof in the manufacture of a medicament for the treatment of Parkinson's disease.

In one embodiment, the invention provides the use of (4-(2-methyl-6-(5-methyl-6-(1-(2-(methylsulfonyl)ethyl)piperidin-4-yl)-1H-indazol-1-yl)pyrimidin-4-yl)morpholin-2-yl)methanol or a pharmaceutically acceptable salt thereof in the manufacture of a medicament for the treatment or prevention of Parkinson's disease, Alzheimer's disease or amyotrophic lateral sclerosis (ALS).

In another embodiment, the invention provides the use of (4-(2-methyl-6-(5-methyl-6-(1-(2-(methylsulfonyl)ethyl)piperidin-4-yl)-1H-indazol-1-yl)pyrimidin-4-yl)morpholin-2-yl)methanol or a pharmaceutically acceptable salt thereof in the manufacture of a medicament for the treatment or prevention of Parkinson's disease.

In yet another embodiment, the invention provides the use of (4-(2-methyl-6-(5-methyl-6-(1-(2-(methylsulfonyl)ethyl)piperidin-4-yl)-1H-indazol-1-yl)pyrimidin-4-yl)morpholin-2-yl)methanol or a pharmaceutically acceptable salt thereof in the manufacture of a medicament for the treatment of Parkinson's disease.

A further aspect of the invention provides a method of treatment or prevention of a disorder listed above (i.e. selected from the neurological diseases and other diseases listed above), which comprises administering to a subject in need thereof a therapeutically effective amount of a compound of Formula (I) or a pharmaceutically acceptable salt thereof. A further aspect of the invention provides a method of treatment or prevention of Parkinson's disease, which comprises administering to a subject in need thereof a therapeutically effective amount of a compound of Formula (I) or a pharmaceutically acceptable salt thereof. A further aspect of the invention provides a method of treatment of Parkinson's disease, which comprises administering to a subject in need thereof a therapeutically effective amount of a compound of Formula (I) or a pharmaceutically acceptable salt thereof. A further aspect of the invention provides a method of treatment or prevention of Alzheimer's disease, which comprises administering to a subject in need thereof a therapeutically effective amount of a compound of Formula (I) or a pharmaceutically acceptable salt thereof. A further aspect of the invention provides a method of treatment of Alzheimer's disease, which comprises administering to a subject in need thereof a therapeutically effective amount of a compound of Formula (I) or a pharmaceutically acceptable salt thereof. A further aspect of the invention provides a method of treatment of tuberculosis, which comprises administering to a subject in need thereof a therapeutically effective amount of a compound of Formula (I) or a pharmaceutically acceptable salt thereof. In an embodiment, the subject is human.

In one embodiment, the invention provides a method of treatment of Parkinson's disease, Alzheimer's disease or amyotrophic lateral sclerosis (ALS), which comprises administering to a subject in need thereof a therapeutically effective amount of a compound of Formula (I) or a pharmaceutically acceptable salt thereof.

In one embodiment, the invention provides a method of treatment of Parkinson's disease, Alzheimer's disease or amyotrophic lateral sclerosis (ALS), which comprises administering to a human in need thereof a therapeutically effective amount of a compound of Formula (I) or a pharmaceutically acceptable salt thereof.

In one embodiment, the invention provides a method of treatment of Parkinson's disease, which comprises administering to a subject in need thereof a therapeutically effective amount of a compound of Formula (I) or a pharmaceutically acceptable salt thereof.

In one embodiment, the invention provides a method of treatment of Parkinson's disease, which comprises administering to a human in need thereof a therapeutically effective amount of a compound of Formula (I) or a pharmaceutically acceptable salt thereof.

In one embodiment, the invention provides a method of treatment of Parkinson's disease, which comprises administering to a human in need thereof a therapeutically effective amount of a compound selected from

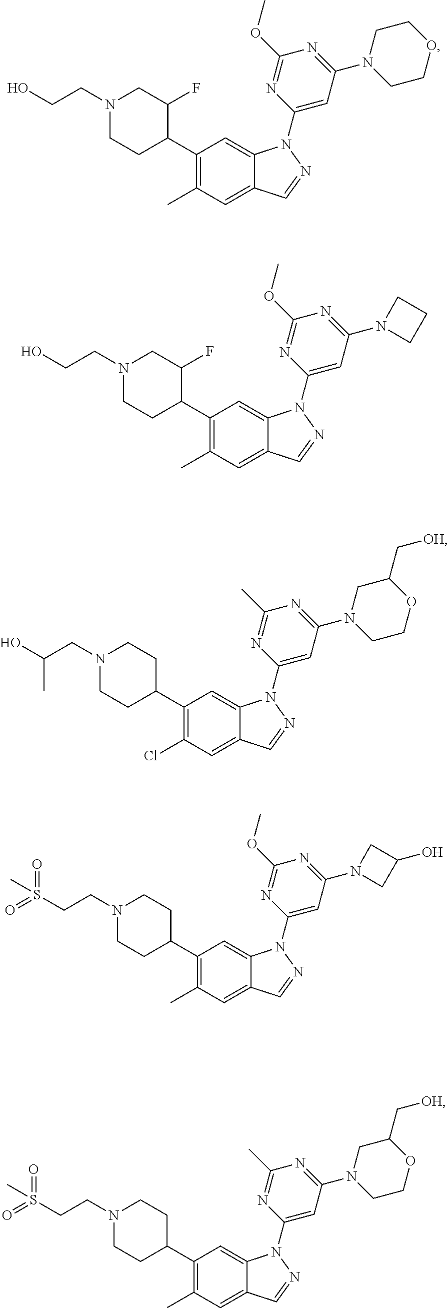 [SMILES: COc1nc(N2CCOCC2)cc(-n2ncc3cc(C)c(C4CCN(CCO)CC4F)cc32)n1 ; COc1nc(N2CCC2)cc(-n2ncc3cc(C)c(C4CCN(CCO)CC4F)cc32)n1 ; Cc1nc(N2CCOC(CO)C2)cc(-n2ncc3cc(Cl)c(C4CCN(CC(C)O)CC4)cc32)n1 ; COc1nc(N2CC(O)C2)cc(-n2ncc3cc(C)c(C4CCN(CCS(C)(=O)=O)CC4)cc32)n1 ; Cc1nc(N2CCOC(CO)C2)cc(-n2ncc3cc(C)c(C4CCN(CCS(C)(=O)=O)CC4)cc32)n1]

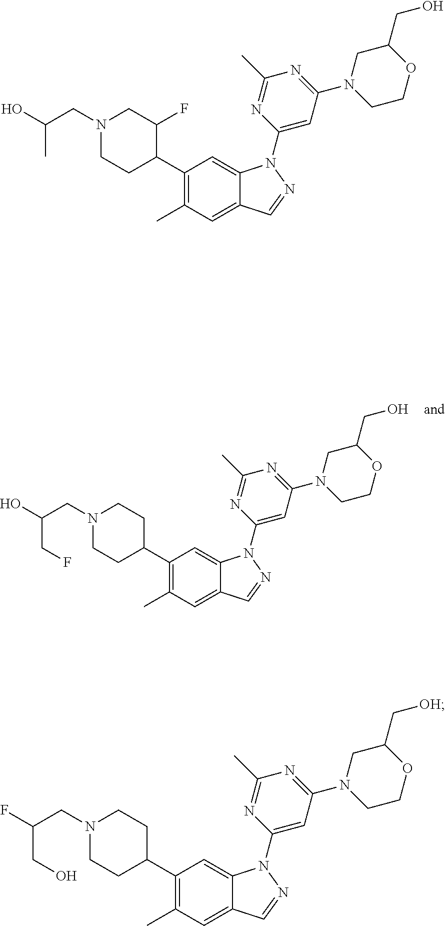 [SMILES: Cc1nc(N2CCOC(CO)C2)cc(-n2ncc3cc(C)c(C4CCN(CC(C)O)CC4F)cc32)n1 ; Cc1nc(N2CCOC(CO)C2)cc(-n2ncc3cc(C)c(C4CCN(CC(O)CF)CC4)cc32)n1 ; Cc1nc(N2CCOC(CO)C2)cc(-n2ncc3cc(C)c(C4CCN(CC(F)CO)CC4)cc32)n1]

or a pharmaceutically acceptable salt thereof.

In one embodiment, the invention provides a method of treatment of Parkinson's disease, which comprises administering to a human in need thereof a therapeutically effective amount of 1-(4-(5-chloro-1-(6-(2-(hydroxymethyl)morpholino)-2-methylpyrimidin-4-yl)-1H-indazol-6-yl)piperidin-1-yl)propan-2-ol.

In one embodiment, the invention provides a method of treatment of Parkinson's disease, which comprises administering to a human in need thereof a therapeutically effective amount of 1-(4-(5-chloro-1-(6-(2-(hydroxymethyl)morpholino)-2-methylpyrimidin-4-yl)-1H-indazol-6-yl)piperidin-1-yl)propan-2-ol or a pharmaceutically acceptable salt thereof.

In one embodiment, the invention provides a method of treatment of Parkinson's disease, which comprises administering to a human in need thereof a therapeutically effective amount of (4-(2-methyl-6-(5-methyl-6-(1-(2-(methylsulfonyl)ethyl)piperidin-4-yl)-1H-indazol-1-yl)pyrimidin-4-yl)morpholin-2-yl)methanol.

In one embodiment, the invention provides a method of treatment of Parkinson's disease, which comprises administering to a human in need thereof a therapeutically effective amount of (4-(2-methyl-6-(5-methyl-6-(1-(2-(methylsulfonyl)ethyl)piperidin-4-yl)-1H-indazol-1-yl)pyrimidin-4-yl)morpholin-2-yl)methanol or a pharmaceutically acceptable salt thereof.

In one embodiment, the invention provides a compound of Formula (I) or a pharmaceutically acceptable salt thereof which is a compound of any one of Examples 1 to 43 or a pharmaceutically acceptable salt thereof. In one embodiment, the invention provides a compound of Formula 1 which is a compound of any one of Examples 1 to 43.

In the context of the present invention, treatment of Parkinson's disease refers to the treatment of sporadic Parkinson's disease, and/or familial Parkinson's disease. In one embodiment, treatment of Parkinson's disease refers to treatment of familial Parkinson's disease. Familial Parkinson's disease patients are those expressing one or more of the following LRRK2 kinase mutations: G2019S mutation, N1437H mutation, R1441G mutation, R1441C mutation, R1441H mutation, Y1699C mutation, S1761R mutation, or 12020T mutation. In another embodiment, familial Parkinson's disease patients express other coding mutations (such as G2385R) or non-coding single nucleotide polymorphisms at the LRRK2 locus that are associated with Parkinson's disease In a more particular embodiment, familial Parkinson's disease includes patients expressing the G2019S mutation or the R1441G mutation in LRRK2 kinase. In one embodiment, treatment of Parkinson's disease refers to the treatment of familial Parkinson's disease includes patients expressing LRRK2 kinase bearing G2019S mutation. In another embodiment, familial Parkinson's disease patients express aberrantly high levels of normal LRRK2 kinase.

In one embodiment, the invention provides a method of treatment of Parkinson's disease, which comprises administering to a human expressing the G2019S mutation in LRRK2 kinase in need thereof a therapeutically effective amount of a compound of Formula (I) or a pharmaceutically acceptable salt thereof.

In one embodiment, the invention provides a method of treatment of Parkinson's disease, which comprises testing in a human for the G2019S mutation in LRRK2 kinase and administering to the human expressing the G2019S mutation in LRRK2 kinase in need thereof a therapeutically effective amount of a compound of Formula (I) or a pharmaceutically acceptable salt thereof.

Treatment of Parkinson's disease may be symptomatic or may be disease modifying. In one embodiment, treatment of Parkinson's disease refers to symptomatic treatment. In one embodiment, treatment of Parkinson's disease refers to disease modifying treatment.

Compounds of the present invention may also be useful in treating patients identified as susceptible to progression to severe Parkinsonism by means of one or more subtle features associated with disease progression such as family history, olfaction deficits, constipation, cognitive defects, gait or biological indicators of disease progression gained from molecular, biochemical, immunological or imaging technologies. In this context, treatment may be symptomatic or disease modifying.

In the context of the present invention, treatment of Alzheimer's disease refers to the treatment of sporadic Alzheimer's disease and/or familial Alzheimer's disease. Treatment of Alzheimer's disease may be symptomatic or may be disease modifying. In one embodiment, treatment of Alzheimer's disease refers to symptomatic treatment.

In the context of the present invention, treatment of dementia (including Lewy body dementia and vascular dementia, HIV-induced dementia), amyotrophic lateral sclerosis (ALS), age related memory dysfunction, mild cognitive impairment, argyrophilic grain disease, Pick's disease, corticobasal degeneration, progressive supranuclear palsy, inherited frontotemporal dementia and parkinsonism linked to chromosome 17 (FTDP-17), multiple sclerosis, lysosomal disorders (for example, Niemann-Pick Type C disease, Gaucher disease), Crohn's disease, cancers (including thyroid, renal (including papillary renal), breast, lung and prostate cancers, leukemias (including acute myelogenous leukemia (AML)) and lymphomas), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, obesity, epilepsy, pulmonary diseases such as chronic obstructive pulmonary disease, idiopathic pulmonary fibrosis, Sjogren's syndrome, Devic's disease, inflammatory myopathies, ankylosing spondylitis, may be symptomatic or disease modifying. In certain embodiments, treatment of these disorders refers to symptomatic treatment.

The invention also provides the use of inhibitors of LRRK2 in the production of neuronal progenitor cells in vitro for consequent therapeutic application in cell based-treatment of CNS disorders.

When a compound of Formula (I) or a pharmaceutically acceptable salt thereof is intended for use in the treatment of Parkinson's disease, it may be used in combination with medicaments alleged to be useful as symptomatic treatments of Parkinson's disease. Suitable examples of such other therapeutic agents include L-dopa, and dopamine agonists (e.g. pramipexole, ropinirole).

When a compound of Formula (I) or a pharmaceutically acceptable salt thereof is intended for use in the treatment of Alzheimer's disease, it may be used in combination with medicaments claimed to be useful as either disease modifying or symptomatic treatments of Alzheimer's disease. Suitable examples of such other therapeutic agents may be symptomatic agents, for example those known to modify cholinergic transmission such as M1 muscarinic receptor agonists or allosteric modulators, M2 muscarinic antagonists, acetylcholinesterase inhibitors (such as tetrahydroaminoacridine, donepezil hydrochloride rivastigmine, and galantamine), nicotinic receptor agonists or allosteric modulators (such as α7 agonists or allosteric modulators or α4β2 agonists or allosteric modulators), PPAR agonists (such as PPARγ agonists), 5-HT₄ receptor partial agonists, 5-HT₆ receptor antagonists e.g. SB-742457 or 5HT1A receptor antagonists and NMDA receptor antagonists or modulators, or disease modifying agents such as β or γ-secretase inhibitors e.g semagacestat, mitochondrial stabilizers, microtubule stabilizers or modulators of Tau pathology such as Tau aggregation inhibitors (e.g. methylene blue and REMBER™), NSAIDS, e.g. tarenflurbil, tramiprosil; or antibodies for example bapineuzumab or solanezumab; proteoglycans for example tramiprosate.

When a compound of Formula (I) or a pharmaceutically acceptable salt thereof is intended for use in the treatment of bacterial infections, parasitic infections or viral infections, it may be used in combination with medicaments alleged to be useful as symptomatic treatments that directly target the infectious agent.

When a compound of Formula (I) or a pharmaceutically acceptable salt thereof is used in combination with other therapeutic agents, the compound may be administered either sequentially or simultaneously by any convenient route.

The invention also provides, in a further aspect, a combination comprising a compound of Formula (I) or a pharmaceutically acceptable salt thereof together with one or more further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of Formula (I) or a pharmaceutically acceptable salt thereof is used in combination with a second therapeutic agent active against the same disease state the dose of each compound may differ from that when the compound is used alone. Appropriate doses will be readily appreciated by those skilled in the art.

### D. Composition

Compounds of Formula (I) or pharmaceutically acceptable salts thereof may be formulated into pharmaceutical compositions prior to administration to a subject. According to one aspect, the invention provides a pharmaceutical composition comprising a compound of Formula (I) or a pharmaceutically acceptable salt thereof and a pharmaceutically acceptable excipient. According to another aspect, the invention provides a process for the preparation of a pharmaceutical composition comprising admixing a compound of Formula (I) or a pharmaceutically acceptable salt thereof, with a pharmaceutically acceptable excipient.

Pharmaceutical compositions may be presented in unit dose forms containing a predetermined amount of active ingredient per unit dose. Such a unit may contain, for example, 0.1 mg, 0.5 mg, or 1 mg to 50 mg, 100 mg, 200 mg, 250 mg, 500 mg, 750 mg or 1 g of a compound of the present invention, depending on the disease being treated, the route of administration and the age, weight and condition of the subject, or pharmaceutical compositions may be presented in unit dose forms containing a predetermined amount of active ingredient per unit dose. In other embodiments, the unit dosage compositions are those containing a daily dose or sub-dose as described herein, or an appropriate fraction thereof, of an active ingredient. Furthermore, such pharmaceutical compositions may be prepared by any of the methods well-known to one skilled in the art.

A therapeutically effective amount of a compound of Formula (I) will depend upon a number of factors including, for example, the age and weight of the intended recipient, the precise condition requiring treatment and its severity, the nature of the formulation, and the route of administration, and will ultimately be at the discretion of the attendant prescribing the medication. However, a therapeutically effective amount of a compound of formula (I) for the treatment of diseases described in the present invention will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or in a number of sub-doses per day as such as two, three, four, five or six doses per day. Or the dosing can be done intermittently, such as once every other day, once a week or once a month. A therapeutically effective amount of a pharmaceutically acceptable salt or solvate, etc., may be determined as a proportion of the therapeutically effective amount of the compound of Formula (I) per se. It is envisaged that similar dosages would be appropriate for treatment of the other diseases referred to above.

The pharmaceutical compositions of the invention may contain one or more compounds of Formula (I) or a pharmaceutically acceptable salt thereof. In some embodiments, the pharmaceutical compositions may contain more than one compound of the invention. For example, in some embodiments, the pharmaceutical compositions may contain two or more compounds of Formula (I) or a pharmaceutically acceptable salt thereof. In addition, the pharmaceutical compositions may optionally further comprise one or more additional active pharmaceutical ingradients (APIs).

As used herein, “pharmaceutically acceptable excipient” means a pharmaceutically acceptable material, composition or vehicle involved in giving form or consistency to the pharmaceutical composition. Each excipient may be compatible with the other ingredients of the pharmaceutical composition when commingled such that interactions which would substantially reduce the efficacy of the compound of the invention when administered to a subject and interactions which would result in pharmaceutical compositions that are not pharmaceutically acceptable are avoided.

The compounds of the invention and the pharmaceutically-acceptable excipient or excipients may be formulated into a dosage form adapted for administration to the subject by the desired route of administration. For example, dosage forms include those adapted for (1) oral administration (including buccal or sublingual) such as tablets, capsules, caplets, pills, troches, powders, syrups, elixers, suspensions, solutions, emulsions, sachets, and cachets; (2) parenteral administration (including subcutaneous, intramuscular, intravenous or intradermal) such as sterile solutions, suspensions, and powders for reconstitution; (3) transdermal administration such as transdermal patches; (4) rectal administration such as suppositories; (5) nasal inhalation such as dry powders, aerosols, suspensions, and solutions; and (6) topical administration (including buccal, sublingual or transdermal) such as creams, ointments, lotions, solutions, pastes, sprays, foams, and gels. Such compositions may be prepared by any methods known in the art of pharmacy, for example by bringing into association a compound of Formula (I) with the carrier(s) or excipient(s). Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Suitable pharmaceutically-acceptable excipients may vary depending upon the particular dosage form chosen. In addition, suitable pharmaceutically-acceptable excipients may be chosen for a particular function that they may serve in the composition. For example, tertain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically acceptable excipients may be chosen for their ability to facilitate carrying or transporting the compound or compounds of the invention once administered to the subject from an organ, or a portion of the body, to another organ, or a portion of the body. Certain pharmaceutically-acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that tertain pharmaceutically-acceptable excipients may serve more than one function and may serve alternative functions depending on how much the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a therapeutically effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate.

The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

In certain embodiments, the present invention is directed to a pharmaceutical composition comprising 0.01 to 1000 mg of one or more of a compound of Formula (I) or a pharmaceutically acceptable salt thereof and 0.01 to 5 g of one or more pharmaceutically acceptable excipients.

In another embodiment, the present invention is directed to a pharmaceutical composition for the treatment of a neurodegeneration disease comprising a compound of formula (I) or a pharmaceutically acceptable salt thereof and a pharmaceutically acceptable excipient. In another embodiment, the present invention is directed to a pharmaceutical composition for the treatment of Parkinson's disease comprising a compound of formula (I) or a pharmaceutically acceptable salt thereof and a pharmaceutically acceptable excipient.

### E. Process of Preparing Compounds

The process to be utilized in the preparation of compounds of formula (I) or salts thereof described herein depends upon the desired compounds. Such factors as the selection of the specific substituent and various possible locations of the specific substituent all play a role in the path to be followed in the preparation of the specific compounds of this invention. Those factors are readily recognized by one of ordinary skill in the art.

In general, the compounds of the present invention may be prepared by standard techniques known in the art and by known processes analogous thereto. General methods for preparing compounds of formula (I) are set forth below. All starting material and reagents described in the below general experimental schemes are commercially available or can be prepared by methods known to one skilled in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

General Scheme 1 provides exemplary processes of synthesis for preparing compounds of the present invention.

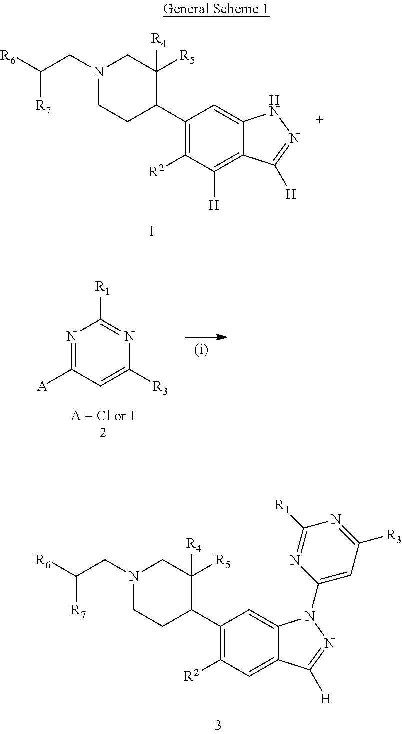

General Scheme 1 provides an exemplary synthesis for preparing compound 3 which represents compounds of Formula (I). In Scheme 1, R₁, R₂, R₃, R₄, R₅, R₆ and R₇ are as defined in Formula I.

Step (i) may be a substitution reaction by reacting compound 1 with compound 2 using appropriate base such as Cs₂CO₃ in an appropriate solvent such as N, N-dimethylformamide (DMF) under suitable temperature such as about 100° C. to provide compound 3.

Step (i) may alternatively be a coupling reaction using appropriate reagents such as CuI and N,N′-dimethyl-cyclohexane-1,2-diamine in the presence of suitable base such as K₃PO₄ in a suitable solvent such as toluene at suitable temperature such as reflux condition to provide compound 3.

Step (i) may alternatively be a coupling reaction using appropriate reagents such as Pd₂dba₃ and di-tert-butyl(2′,4′,6′-triisopropyl-[1,1′-biphenyl]-2-yl)phosphine in the presence of suitable base such as sodium tert-butoxide in a suitable solvent such as toluene at suitable temperature such as 100° C. to provide compound 3.

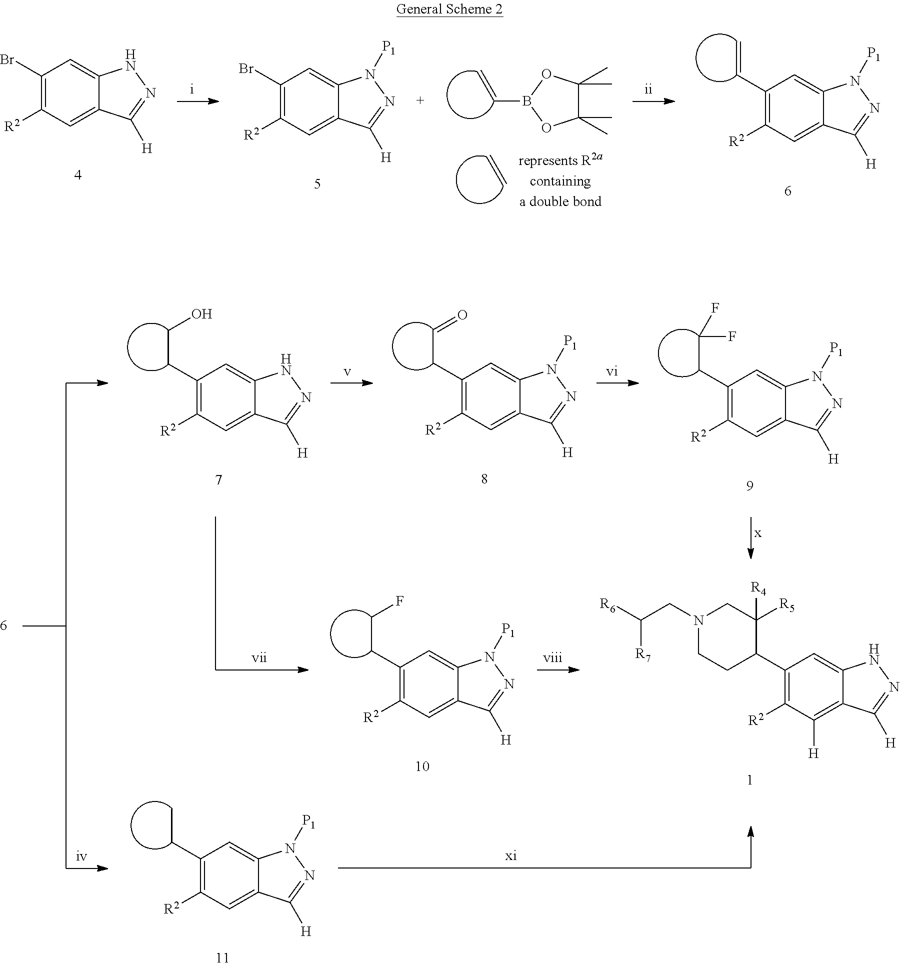

General Scheme 2 provides an exemplary synthesis for preparing intermediate 1. The protecting group, P₁, can be any suitable protecting groups for example, tetrahydro-2H-pyran-2-yl (THP), (trimethylsilyl)ethoxy)methyl (SEM) or or Acetyl (Ac).

Intermediate 5 can be obtained in step (i) by reacting starting material 4 with suitable reagents such as DHP in the presence of suitable acids such as TsOH in appropriate solvents such as DCM under suitable temperatures such as 20° C. to 40° C.

Step (ii) is a cross-coupling reaction between intermediate 5 and boronic acid or esters using appropriate palladium catalysts such as Pd(dppf)Cl₂ in the presence of suitable bases such as Na₂CO₃ in appropriate solvents such as 1,4-dioxane at suitable temperatures such as 60° C. to 100° C.

Step (iii) involves reaction with suitable oxidation reagents such as H₂O₂ in a suitable solvent such as THF under suitable temperatures such as −60° C. to −10° C. to provide intermediate 7.

Step (iv) is a reaction with a suitable reducing reagent such as hydrogen in the presence of suitable catalysts such Pd/C in polar solvents such as MeOH at appropriate temperatures such as 25° C. to 80° C.

Step (v) may be an oxidation reaction with oxidants such as DMP in suitable solvents such as DCM under suitable temperatures such as 0° C. to 25° C. to give intermediate 8.

Steps (vi) and (viii) involve reaction with a fluridizer such as DAST in suitable solvents such as DCM under suitable temperatures such as −78° C. to 0° C.

Steps (viii) (x) and (xi) are de-protection reactions. Typically, the intermediate is reacted with suitable acids such HCl in suitable solvents such as 1,4-dioxane under suitable temperatures such as 25° C. to 40° C. to give intermediate 1.

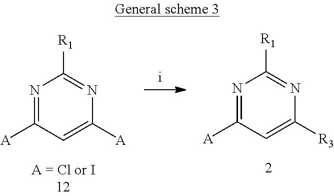

General Scheme 3 provides an exemplary synthesis for preparing intermediate 2.

When R₃ is an N-linked 4-6 membered heterocyclyl ring or NHR⁷; step (i) can be a reaction with different amines using appropriate bases such as TEA in appropriate solvents such as EtOH under suitable temperatures such as 25° C. to 100° C. to provide intermediate 2.

When R₃ is OR⁷, step (i) is a coupling reaction. The alcohol (R⁷OH) is deprotonated by a suitable base such as sodium hydride in suitable solvent such as THF at a suitable temperature such as 0° C. to give the transitional intermediate. Then intermediate 12 is reacted with the transitional intermediate in suitable solvent such as THF at suitable temperature such as room temperature.

### EXAMPLES

### General Experimental Procedures

The following descriptions and examples illustrate the invention. These examples are not intended to limit the scope of the present invention, but rather to provide guidance to the skilled chemist to prepare and use the compounds, compositions and methods of the present invention. While particular embodiments of the present invention are described, the skilled chemist will appreciate that various changes and modifications can be made without departing from the spirit and scope of the invention.

The chemical names of compounds described in the present application were generally created from ChemDraw Ultra (ChambridgeSoft) and/or generally follow the principle of IUPAC nomenclature.

Heating of reaction mixtures with microwave irradiations was carried out on a Smith Creator (purchased from Personal Chemistry, Forboro/MA, now owned by Biotage), an Emrys Optimizer (purchased from Personal Chemistry) or an Explorer (provided by CEM Discover, Matthews/NC) microwave.

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the compounds in the examples may be carried out by conventional methods such as chromatography and/or re-crystallization using suitable solvents. Chromatographic methods are known to the skilled person and include e.g. column chromatography, flash chromatography, HPLC (high performance liquid chromatography), and MDAP (mass directed auto-preparation, also referred to as mass directed LCMS purification). MDAP is described in e.g. W. Goetzinger et al, Int. J. Mass Spectrom., 2004, 238, 153-162.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. Preparative HPLC were performed using a Gilson Preparative System using a Luna 5 u C18(2) 100A reverse phase column eluting with a 10-80 gradient (0.1% FA in acetonitrile/0.1% aqueous FA) or a 10-80 gradient (acetonitrile/water). The CombiFlash system used for purification in this application was purchased from Isco, Inc. CombiFlash purification was carried out using a pre-packed SiO₂ column, a detector with UV wavelength at 254 nm and mixed solvents.

The terms “CombiFlash”, “Biotage®”, “Biotage 75” and “Biotage SP4®” when used herein refer to commercially available automated purification systems using pre-packed silica gel cartridges.

Final compounds were characterized with LCMS (conditions listed below) or NMR. ¹H NMR or ¹⁹FNMR spectra were recorded using a Bruker Avance 400 MHz spectrometer. CDCl₃ is deuteriochloroform, DMSO-d₆ is hexadeuteriodimethylsulfoxide, and CD₃OD is tetradeuteriomethanol. Chemical shifts are reported in parts per million (ppm) downfield from the internal standard tetramethylsilane (TMS) or the NMR solvent. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz.

All temperatures are reported in degrees Celsius. All other abbreviations are as described in the ACS Style Guide (American Chemical Society, Washington, D.C., 1986).

Absolute stereochemistry can be determined by methods known to one skilled in the art, for example X-ray or Vibrational Circular Dichroism (VCD).

When an enantiomer or a diasteroisomer is described and the absolute stereochemistry of a chiral center is not known, the use of “*” at the chiral centre denotes that the absolute stereochemistry of the chiral center is not known, i.e. the compound as drawn may be either a single R enantiomer or a single S enantiomer. Where the absolute stereochemistry at a chiral center of an enantiomer or a diasteroisomer is known, a bold wedge symbol ( [symbol] ) or a hashed wedge symbol ( [symbol] ) is used as appropriate, without the use of “*” at the chiral centre.

When a geometric or cis-trans isomer is described and the absolute configuration of the isomer is not known, the use of “*” at one of the atoms relevant to the geometric or cis-trans isomerism denotes that the absolute configuration at or around that atom is not known, i.e. the compound as drawn may be either a single cis isomer or a single trans enantiomer.

In the procedures that follow, after each starting material, reference to an intermediate is typically provided. This is provided merely for assistance to the skilled chemist. The starting material may not necessarily have been prepared from the batch referred to.

### LCMS Conditions:

1) Acidic method:
a. Instruments: HPLC: Waters UPC2 and MS: Qda
Mobile phase: water containing 0.1% FA/0.1% MeCN

### Column: ACQUITY UPLC BEH C₁₈ 1.7 μm 2.1×50 mm and 1.7 μm 2.1×100 mm

Detection: MS and photodiode array detector (PDA)
b. Instruments: HPLC: Shimadzu and MS: 2020
Mobile phase: water containing 0.1% FA/0.1% MeCN
Column: Sunfire C₁₈ 5 μm 50×4.6 mm and Sunfire C₁₈ 5 μm 150×4.6 mm
Detection: MS and photodiode array detector (PDA)
2) Basic conditions:

### Instruments: HPLC: Agilent 1260 and MS: 6120

Mobile phase: 0.1% NH₄OH in H₂O/0.1% NH₄OH in ACN
Column: Xbridge C₁₈ 5 μm 50×4.6 mm and Xbridge C₁₈ 5 μm 150×4.6 mm
Detection: MS and photodiode array detector (DAD)
Prep-HPLC conditions
Instrument: Waters instrument
Column: Xbridge Prep C₁₈ column OBD (10 μm, 19×250 mm), Xbrige prep C₁₈ 10 μm OBD™ 19×150 mm, Sunfire Prep C₁₈ 10×25 0 mm 5 μm, XBRIDGE Prep C₁₈ 10×150 mm 5 μm, etc
Acidic method:
Mobile phase: water containing 0.1% TFA/acetonitrile.
Basic method:
Mobile phase: water containing 0.1% NH₄OH/acetonitrile.
Chiral prep-HPLC:
Thar SFC Prep 80 (TharSFC ABPR1, TharSFC SFC Prep 80 CO₂ Pump, TharSFC Co-Solvent Pump, TharSFC Cooling Heat Exchanger and Circulating Bath, TharSFC Mass Flow Meter, TharSFC Static Mixer, TharSFC Injection Module, Gilson UV Detector, TharSFC Fraction Collection Module
Chiral-HPLC analysis:
Instrument: Thar SFC Prep 80 (TharSFC ABPR1, TharSFC SFC Prep 80 CO₂Pump, TharSFC Co-Solvent Pump, TharSFC Cooling Heat Exchanger and Circulating Bath, TharSFC Mass Flow Meter, TharSFC Static Mixer, TharSFC Injection Module, Gilson UV Detector, TharSFC Fraction Collection Module

Column and mobile phase: are described in below examples.

### Abbreviations and Resource Sources

The following abbreviations and resources are used herein below:

Ac—acetyl
MeCN—acetonitrile
Atm—atmosphere
Aq.—aqueous
BINAP—2,2′-bis(diphenylphosphino)-1,1′-binaphthyl
Boc—tert-butyloxycarbonyl
Boc₂O—di-tert-butyl dicarbonate
Bn—benzyl
t-Bu—tert-butyl
conc.—concentrated
DAST—N,N-diethylaminosulfur trifluoride
DCE—1,2-dichloroethane
DCM—dichloromethane
DEA—diethanolamine

### DMEDA—N,N′-Dimethylethylenediamine

Dess-Martin—1,1,1-Tris(acetyloxy)-1,1-dihydro-1,2-benziodoxol-3-(1H)-one
DHP—3,4-dihydro-2H-pyran
DIBAL-H—diisobutylaluminum hydride

### DIEA—N,N-diisopropylethylamine

### DIPEA—N, N-diisopropylethylamine

### DMA—N, N-dimethylacetamide

DMAP—4-dimethylaminopyridine
DMEDA—N,N′-dimethylethylenediamine

### DMF—N, N-dimethylformamide

DMP—Dess-Martin periodinane
DMSO—dimethyl sulfoxide
DPPF—1,1′-bis(diphenylphosphino)ferrocene
EA—ethyl acetate
EDC—1-ethyl-3-(3-dimethylaminopropyl)carbodiimide hydrochloride
EDCI—3-(ethyliminomethyleneamino)-N,N-dimethylpropan-1-amine
EtOH/EtOH—ethanol
Et₂O—diethyl ether
EtOAc—ethyl acetate
Et₃N—triethylamine
FA—formic acid
HEP—heptane
Hex—hexane
HOAc—acetic acid
HATU—2-(1H-7-azabenzotriazol-1-yl)-1,1,3,3-tetramethyl uranium hexafluorophosphate
HOBT—hydroxybenzotriazole
IPA—isopropyl alcohol
ⁱPrOH/iPrOH—isopropyl alcohol
m-CPBA—meta-chloroperoxybenzoic acid
MOMCl—monochlorodimethyl ether
Me—methyl
MeOH—methanol
MsCl—methanesulfonyl chloride
NaHMDS—sodium bis(trimethylsilyl)amide

### NIS—N-iodosuccinimide

NMP—1-methyl-2-pyrrolidone
NMO—4-methylmorpholine 4-oxide
PE—petroleum ether
PMB—p-methoxybenzyl
Pd₂(dba)₃—Tris(dibenzylideneacetone)dipalladium

### Pd(dppf)Cl₂—1,1′-Bis(diphenylphosphino)ferrocenepalladium(II)dichloride

dichloromethane complex
Ph₃P—triphenyiphosphine

### PhNTf₂—N,N-bis-(Trifluoromethanesulfonyl)aniline

PPTS—pyridinium p-toluenesulfonate
PTSA—p-toluenesulfonic acid
rt/RT—room temperature
Rt—retention time
sat.—saturated
SEM-Cl—2-(trimethylsilyl)ethoxymethyl chloride

### SFC—Supercritical Fluid Chromatography

TBAl—Tetrabutylammonium iodide
TBDPSCl—tert-Butyl(chloro)diphenylsilane
TEA—triethylamine
TFA—trifluoroacetic acid
TFAA—trifluoroacetic anhydride
THF—tetrahydrofuran
TLC—thin layer chromatography
TsCl—4-toluenesulfonyl chloride
TsOH—p-toluenesulfonic acid

### Description 1

### 6-Bromo-5-methyl-1H-indazole (D1)

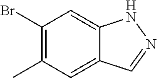

To a solution of 5-bromo-2,4-dimethylaniline (15.0 g, 75.0 mmol) in chloroform (150 mL) was added Ac₂O (15.0, 150 mmol) under ice bath. KOAc (8.00 g, 82.5 mmol), 18-crown-6 (10.0 g, 37.5 mmol) and isoamyl nitrite (26.3 g, 225 mmol) were added. The mixture was refluxed for 36 hrs. The reaction mixture was concentrated and the residue was dissolved in EtOAc (500 mL). The organic solution was washed with water (100 mL), dried over Na₂SO₄ and concentrated. The residue was dissolved in THF (100 mL) and NaOH (4 M, 40.0 mL, 160 mmol) was added. The mixture was stirred at rt for 1 h. The solvent was removed under vacuum and the residue was partitioned between EtOAc (400 mL) and water (200 mL). The organic layer was washed with brine, dried over Na₂SO₄ and concentrated. The crude was purified by column chromatography (PE:EtOAc from 10:1 to 5:1) to give the title compound (5.1 g, yield 32%) as an orange solid.

¹H NMR (300 MHz, CDCl₃): δ 10.20 (br s, 1H), 7.99 (s, 1H), 7.75 (s, 1H), 7.61 (s, 1H), 2.50 (s, 3H).

### Description 2

### 6-Bromo-5-methyl-1-(tetrahydro-2H-pyran-2-yl)-1H-indazole (D2)

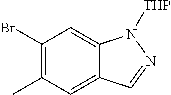

To a solution of 6-bromo-5-methyl-1H-indazole (5.10 g, 24.2 mmol) in dry DCM (120 mL) was added DHP (4.10 g, 48.4 mmol), TsOH (0.800 g, 4.80 mmol) and Mg₂SO₄ (5.0 g) at rt. The reaction mixture was heated to 35° C. and stirred for an hour. The reaction mixture was filtered and the filtrate was washed with a solution of Na₂CO₃ (10%, 100 mL), dried over Na₂SO₄ and concentrated. The crude was purified by column chromatography (PE:EtOAc from 50:1 to 20:1) to give the title compound (6.0 g, yield 84%) as an orange solid.

¹H NMR (300 MHz, CDCl₃): δ 7.90 (s, 1H), 7.84 (s, 1H), 7.55 (s, 1H), 5.63 (dd, J=9.6, 3.0 Hz, 1H), 4.05-4.00 (m, 1H), 3.78-3.70 (m, 1H), 2.58-2.44 (m, 4H), 2.20-2.02 (m, 2H), 1.78-1.65 (m, 3H).

LCMS: (mobile phase: 5-95% CH₃CN), Rt=2.19 min in 3 min; MS Calcd: 294; MS Found: 295 [M+H]⁺.

### Description 3

### Tert-butyl 4-(5-methyl-1-(tetrahydro-2H-pyran-2-yl)-1H-indazol-6-yl)-5,6-dihydropyridine-1(2H)-carboxylate (D3)

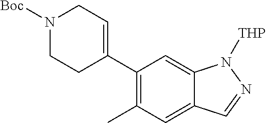

To a suspension of 6-bromo-5-methyl-1-(tetrahydro-2H-pyran-2-yl)-1H-indazole (5.50 g, 18.6 mmol), tert-butyl 4-(4,4,5,5-tetramethyl-1,3,2-dioxaborolan-2-yl)-5,6-dihydropyridine-1(2H)-carboxylate (6.90 g, 22.3 mmol) and Na₂CO₃ (4.90 g, 46.5 mmol) in dioxane (150 mL) and water (130 mL) was added Pd(dppf)Cl₂ (658 mg, 0.900 mmol). The mixture was degassed with N₂ for 3 times and then stirred at 80° C. overnight. The solvent was removed under vacuum and the residue was partitioned between EtOAc (300 mL) and water (200 mL). The combined organic layers were washed with brine, dried over Na₂SO₄ and concentrated. The crude was purified by column chromatography (PE:EtOAc=10:1) to give the title compound (7.30 g, yield 99%) as a slight brown solid.

¹H NMR (400 MHz, CDCl₃): δ 7.92 (s, 1H), 7.48 (s, 1H), 7.28 (s, 1H), 5.67 (dd, J=9.6, 2.8 Hz, 1H), 5.63 (br s, 1H), 4.07-4.01 (m, 3H), 3.78-3.70 (m, 1H), 3.67-3.64 (m, 2H), 2.62-2.53 (m, 1H), 2.45-2.39 (m, 2H), 2.34 (s, 3H), 2.18-2.12 (m, 1H), 2.07-2.02 (m, 1H), 1.81-1.73 (m, 2H), 1.69-1.61 (m, 1H), 1.52 (s, 9H).

### Description 4

### Trans-tert-butyl 3-hydroxy-4-(5-methyl-1-(tetrahydro-2H-pyran-2-yl)-1H-indazol-6-yl)piperidine-1-carboxylate (D4)

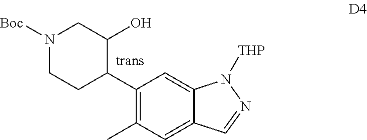

To a solution of tert-butyl 4-(5-methyl-1-(tetrahydro-2H-pyran-2-yl)-1H-indazol-6-yl)-5,6-dihydropyridine-1(2H)-carboxylate (21.0 g, 52.83 mmol) in dry THF (200 mL) was added BH₃-THF solution (1 M, 211 mL, 211 mmol) under N₂ and below 5° C. with internal temperature. The mixture was warmed to rt and stirred overnight. TLC showed the starting material was consumed. After cooled to 0° C., NaOH (aq, 2 M, 79 mL, 158 mmol) was added dropwise carefully and the internal temperature was kept below 10° C. Then H₂O₂ (30%, 20.0 mL, 151 mmol) was added dropwise and the internal temperature was still kept below 10° C. The mixture was stirred at rt. for an hour, then quenched with 150 mL of 10% Na₂S₂O₃ solution under ice bath and stirred for 20 min. The solvent was removed and the residue was extracted with EtOAc (200 mL×2). The combined organic layers were washed with brine, dried over Na₂SO₄ and concentrated. The residue was purified by column chromatography (PE:EtOAc from 10:1 to 2:1) to give the title compound (16.5 g, yield 75%) as a white solid.

¹H NMR (300 MHz, CDCl₃): δ 7.92 (s, 1H), 7.53 (s, 1H), 7.42 (s, 1H), 5.70-5.67 (m, 1H), 4.49-4.44 (m, 1H), 4.30-4.17 (m, 1H), 4.05-3.91 (m, 2H), 3.82-3.72 (m, 1H), 3.04-2.96 (m, 1H), 2.86-2.72 (m, 2H), 2.63-2.53 (m, 1H), 2.47 (s, 3H), 2.21-2.16 (m, 1H), 2.07-2.02 (m, 1H), 1.99-1.67 (m, 6H), 1.52 (s, 9H).

### Description 5

### (cis)-tert-Butyl 3-fluoro-4-(5-methyl-1-(tetrahydro-2H-pyran-2-yl)-1H-indazol-6-yl) piperidine-1-carboxylate (D5)

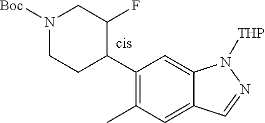

To a solution of (trans)-tert-Butyl 3-hydroxy-4-(5-methyl-1-(tetrahydro-2H-pyran-2-yl)-1H-indazol-6-yl)piperidine-1-carboxylate (24.5 g, 59.0 mmol) in dry DCM (200 mL) was added DAST (38.0 g, 236 mmol) under N₂ at −65° C. The mixture was gradually warmed to rt and stirred for 2 hrs. The reaction mixture was carefully poured into Na₂CO₃ aqueous solution (10%, 300 mL) and stirred for 20 min. The organic layer was separated and the aqueous was extracted with DCM (250 mL×2). The combined organic layers were washed with brine, dried over Na₂SO₄ and evaporated. The crude was purified by column chromatography (PE:EtOAc=10:1) to give the title compound (11.8 g, yield 48%) as a white solid.

¹H NMR (400 MHz, CDCl₃): δ 7.92 (s, 1H), 7.52 (s, 1H), 7.41 (s, 1H), 5.74-5.67 (m, 1H), 4.80-4.59 (m, 2H), 4.21 (br s, 1H), 4.07-3.99 (m, 1H), 3.80-3.71 (m, 1H), 3.25-3.19 (m, 1H), 2.89-2.79 (m, 2H), 2.65-2.51 (m, 1H), 2.45 (s, 3H), 2.19-2.15 (m, 1H), 2.15-2.04 (m, 1H), 1.93-1.88 (m, 1H), 1.80-1.74 (m, 5H), 1.52 (s, 9H).

LCMS (5-95% CH₃CN): Rt=2.25 min in 3 min; MS Calcd: 417; MS Found: 418 [M+H]⁺.

### Description 6

### ((cis)-6-(3-Fluoropiperidin-4-yl)-5-methyl-1-(tetrahydro-2H-pyran-2-yl)-1H-indazole (D6)

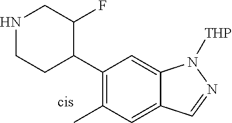

To a solution of (cis)-tert-butyl 3-fluoro-4-(5-methyl-1-(tetrahydro-2H-pyran-2-yl)-1H-indazol-6-yl) piperidine-1-carboxylate (1.60 g, 3.84 mmol) in CH₃OH (10 mL) was added HCl/CH₃OH (5 M, 20 mL). The mixture was stirred at 0° C. for 1 h. The reaction mixture was poured into sat. NaHCO₃ solution (200 mL). The mixture was extracted with EtOAc (50 mL×3). The combined organic layers were washed with brine (50 mL), dried over Na₂SO₄ and concentrated. The residue was purified by column C₁₈ (5%-60% CH₃CN in water) to give the title compound (600 mg, yield 49%) as a yellow oil.

LCMS (mobile phase: 5-95% Acetonitrile in 2.5 min): Rt=1.46 min; MS Calcd: 317; MS Found: 318 [M+H]⁺.

### Description 7

### (cis)-6-(3-Fluoropiperidin-4-yl)-5-methyl-1H-indazole hydrochloride (D7)

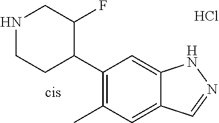 [SMILES: Cc1cc2cn[nH]c2cc1C1CCNCC1F ; Cl]

A mixture of (cis)-tert-butyl 3-fluoro-4-(5-methyl-1-(tetrahydro-2H-pyran-2-yl)-1H-indazol-6-yl) piperidine-1-carboxylate (2.50 g, 6.00 mmol) in HCl/dioxane (6 mol/L, 40 mL) was stirred at rt for 6 hrs. The reaction mixture was cooled to 0° C. and filtered. The solid was washed with cold 1,4-dioxane (5 mL) to get the title compound (1.4 g, yield 100%) as a white solid which was used for next step directly.

LC-MS (5-95% CH₃CN): Rt=1.73 min; MS Calcd.: 233, MS Found: 234 [M+H]⁺.

### Description 8

### (cis)-tert-Butyl 3-fluoro-4-(5-methyl-1H-indazol-6-yl)piperidine-1-carboxylate (D8)

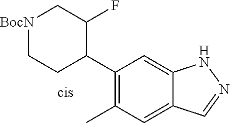 [SMILES: Cc1cc2cn[nH]c2cc1C1CCN(C(=O)OC(C)(C)C)CC1F]

To a solution of (cis)-6-(3-fluoropiperidin-4-yl)-5-methyl-1H-indazole hydrochloride (500 mg, 2.14 mmol) in CH₃OH (5 mL) and H₂O (1 mL) was added KOH (242 mg, 4.29 mmol) and (Boc)₂O (700 mg, 3.21 mmol) under ice bath. The reaction mixture was stirred at rt for 2 hrs. The reaction mixture was diluted with water (30 mL) and extracted with EtOAc (3×20 mL). The combined organic layers were concentrated and purified by column chromatograph (PE:EtOAc=20:1) to give the title compound (180 mg, yield 25%) as colorless oil.

¹H NMR (300 MHz, CDCl₃): δ 9.98 (s, 1H), 7.96 (s, 1H), 7.56 (s, 1H), 7.39 (s, 1H), 4.76-4.54 (m, 2H), 4.27-4.10 (m, 1H), 3.25-3.14 (m, 1H), 2.91-2.76 (m, 2H), 2.48 (s, 3H), 1.97-1.84 (m, 1H), 1.71-1.62 (m, 1H), 1.51 (s, 9H).

### Descriptions 9 and D10

### (cis)-tert-Butyl 3-fluoro-4-(5-methyl-1H-indazol-6-yl)piperidine-1-carboxylate (single cis isomer 1) (D9) and (cis)-tert-Butyl 3-fluoro-4-(5-methyl-1H-indazol-6-yl)piperidine-1-carboxylate (single cis isomer 2) (D10)

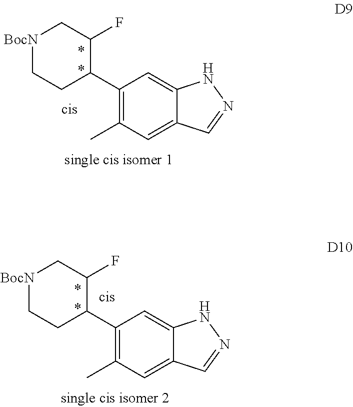 [SMILES: Cc1cc2cn[nH]c2cc1C1CCN(C(=O)OC(C)(C)C)CC1F ; Cc1cc2cn[nH]c2cc1C1CCN(C(=O)OC(C)(C)C)CC1F]

(cis)-tert-Butyl 3-fluoro-4-(5-methyl-1H-indazol-6-yl)piperidine-1-carboxylate (140 mg, 0.420 mmol) was separated by chiral prep. HPLC with the method (Chiralpak IB 5 μm 20*250 nm, Hex: i-PrOH=80:20, Flow: 20 mL/min, 205 nm, T=30° C.) to give (cis)-tert-butyl 3-fluoro-4-(5-methyl-1H-indazol-6-yl)piperidine-1-carboxylate (enantiomer 1) (D9) (68 mg, yield 48%) as a white solid and (cis)-tert-Butyl 3-fluoro-4-(5-methyl-1H-indazol-6-yl)piperidine-1-carboxylate (enantiomer 2) (D10) (47 mg, yield 33%) as a white solid.

single cis isomer 1, D9:

LCMS (mobile phase: 5-95% Acetonitrile in 2.5 min): Rt=1.64 min; MS Calcd: 333 MS Found: 332 [M−H]⁻.

¹H NMR (300 MHz, CDCl₃): δ 10.07 (s, 1H), 7.97 (s, 1H), 7.56 (s, 1H), 7.39 (s, 1H), 4.78-4.53 (m, 2H), 4.32-4.12 (m, 1H), 3.26-3.13 (m, 1H), 2.93-2.75 (m, 2H), 2.47 (s, 3H), 1.94-1.79 (m, 1H), 1.69-1.60 (m, 1H), 1.49 (s, 9H).

Chiral HPLC (Chiralpak IB 5 μm 4.6×250 mm, Phase: Hex/IPA=80/20, flow rate: 1 mL/min, temperature: 30° C.); Rt=6.142 min, 100% ee.

single cis isomer 2, D10:

LCMS: (mobile phase: 5-95% Acetonitrile in 2.5 min), Rt=1.64 min; MS Calcd: 333 MS Found: 332 [M−H]⁻.

¹H NMR (300 MHz, CDCl₃): δ 10.45 (s, 1H), 7.97 (s, 1H), 7.56 (s, 1H), 7.39 (s, 1H), 4.75-4.55 (m, 2H), 4.26-4.16 (m, 1H), 3.24-3.17 (m, 1H), 2.90-2.74 (m, 2H), 2.46 (s, 3H), 1.93-1.87 (m, 1H), 1.70-1.61 (m, 1H), 1.50 (s, 9H).

Chiral HPLC (Chiralpak IB 5 μm 4.6×250 mm, Phase: Hex/IPA=80/20, flowrate: 1 mL/min, temperature: 30° C.): Rt=7.671 min, 100% ee

### Description 11

### 6-((3S,4R)-3-fluoropiperidin-4-yl)-5-methyl-1H-indazole (D11)

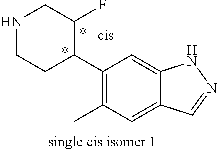 [SMILES: Cc1cc2cn[nH]c2cc1C1CCNCC1F]

To a solution of tert-butyl 3-fluoro-4-(5-methyl-1H-indazol-6-yl)piperidine-1-carboxylate (D9, 100 mg, 0.30 mmol), in MeOH (1.5 mL) was added HCl/MeOH (5M, 1 mL) at 0° C. The reaction mixture was warmed to room temperature and stirred overnight. The solvent was removed under vacuum and Na₂CO₃ solution (5 mL) was added. It was extracted with EtOAc for 3 times. The organic phase was combined, dried, filtered and concentrated to give the crude product as a white solid.

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=0.49 min; MS Calcd.: 233, MS Found: 234 [M+H]⁺.

### Description 12

### 6-((3S,4R)-3-fluoropiperidin-4-yl)-5-methyl-1H-indazole (D12)

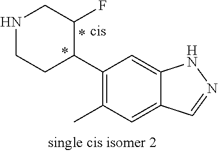 [SMILES: Cc1cc2cn[nH]c2cc1C1CCNCC1F]

The title compound was prepared by a procedure similar to those described for D11 starting from a suspension of 6-((3R,4S)-3-fluoropiperidin-4-yl)-5-methyl-1H-indazole (D10), 1-bromo-2-fluoroethane, K₂CO₃ in DMF.

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=0.29 min; MS Calcd.: 279.1, MS Found: 280.2 [M+H]⁺.

### Description 13

### 4,6-Diiodo-2-methoxypyrimidine (D13)

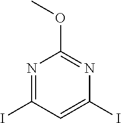

To a solution of NaI (1.10 g, 7.34 mmol) in HI (55%, 7.5 mL) was added 4,6-dichloro-2-methoxypyrimidine (1.00 g, 5.59 mmol). The mixture was heated to 40° C. and stirred for 10 h. The reaction mixture was cooled to room temperature, poured into ice water (50 mL) and filtered to give the crude solid. The residue was purified by column chromatography (PE:EtOAc=10:1) to give the title product (640 mg, yield 31.7%) as a white solid.

¹H NMR (400 MHz, CDCl₃): δ 7.85 (s, 1H), 4.00 (s, 3H).

### Description 14

### 4-(6-iodo-2-methoxypyrimidin-4-yl)morpholine (D14)

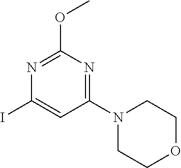

A mixture of 4,6-diiodo-2-methoxypyrimidine (1.00 g, 2.80 mol) and morpholine (240 mg, 2.80 mol) in Et₃N (850 g, 8.40 mmol) and EtOH (20 mL) was stirred at RT overnight. The reaction solution was poured into sat. NH₄Cl (50 mL) and extracted with EtOAc (3×60 mL). The combined organic layers were dried, filtered and concentrated. The residue was purified by chromatography (PE:EtOAc=3:1) to give the title product (850 mg, yield: 95%) as a white solid.

LC-MS [mobile phase: from 80% water (0.1% FA) and 20% CH₃CN (0.1% FA) to 5% water (0.1% FA) and 95% CH₃CN (0.1% FA) in 2.0 min]: Rt=0.89 min; MS Calcd.: 321.0, MS Found: 322.2 [M+H]⁺.

### Description 15

### (R)-4-(6-iodo-2-methoxypyrimidin-4-yl)-3-methylmorpholine (D15)

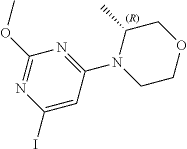

To a solution of 4,6-diiodo-2-methoxypyrimidine (725 mg, 2.00 mmol) and (R)-3-methylmorpholine hydrochloride (202 mg, 2.00 mmol) in i-PrOH/THF (10 mL/10 mL) was added DIEDA (776 mg, 6 mmol). The mixture was stirred at 80° C. overnight, then concentrated to give the residue. The crude was purified by chromatography (PE:EtOAc=7:1) to give the title product as a colorless oil (500 mg, yield: 75%) which was directly used into next step.

### Description 16

### tert-butyl cis-3-fluoro-4-(1-(2-methoxy-6-((R)-3-methylmorpholino)pyrimidin-4-yl)-5-methyl-1H-indazol-6-yl)piperidine-1-carboxylate (D16)

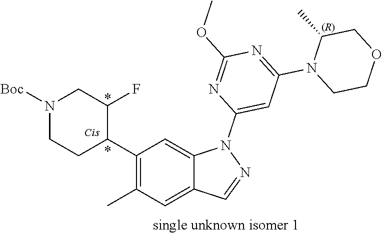

To a mixture of tert-butyl cis-3-fluoro-4-(5-methyl-1H-indazol-6-yl)piperidine-1-carboxylate (D9, 80 mg, 0.24 mmol) and (R)-4-(6-iodo-2-methylpyrimidin-4-yl)-3-methylmorpholine (87 mg, 0.26 mmol) in toluene/THF (5 mL/1 mL) were added N¹,N²-dimethylethane-1,2-diamine (32 mg, 0.31 mmol), CuI (51 mg, 0.24 mmol) and K₃PO₄ (110 mg, 0.48 mmol). The reaction mixture was stirred at 90° C. for 2 hours under N₂, diluted with aq.NH₃.H₂O (50 mL) and extracted with EtOAc (50 mL×3). The combined organic layers were dried, filtered and concentrated. The purification by chromatography (PE:EtOAc=5:1) afforded the title product as a white oil (125 mg, yield: 96%).

LC-MS [mobile phase: 40% water (0.1% FA) and 60% CH₃CN (0.1% FA) to 5% water (0.1% FA) and 95% CH₃CN (0.1% FA) in 9.0 min]: Rt=4.56 min; MS Calcd.: 540.6, MS Found: 541.4 [M+H]⁺.

### Description 17

### cis-(3R)-4-(6-(6-(3-fluoropiperidin-4-yl)-5-methyl-1H-indazol-1-yl)-2-methoxypyrimidin-4-yl)-3-methylmorpholine hydrochloride (D17)

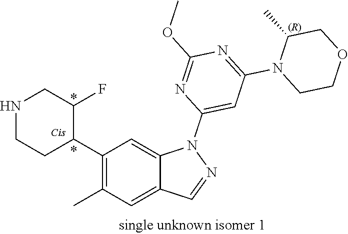

To a solution of tert-butyl cis-3-fluoro-4-(1-(2-methoxy-6-((R)-3-methylmorpholino) pyrimidin-4-yl)-5-methyl-1H-indazol-6-yl)piperidine-1-carboxylate (D16, 125 mg, 0.200 mmol) in EtOAc (5 mL) was slowly dropped HCl.EtOAc (2.00 mL, 3.50 mol/L) in ice bath. The reaction mixture was stirred at rt. for 30 min, then concentrated to give the crude as a white solid (80 mg).

LC-MS [mobile phase: 90% water (0.1% FA) and 10% CH₃CN (0.1% FA) to 5% water (0.1% FA) and 95% CH₃CN (0.1% FA) in 9.0 min]: Rt=4.83 min; MS Calcd.: 440.5, MS Found: 441.3 [M+H]⁺.

### Description 18

### ethyl cis-2-(3-fluoro-4-(1-(2-methoxy-6-((R)-3-methylmorpholino)pyrimidin-4-yl)-5-methyl-1H-indazol-6-yl)piperidin-1-yl)acetate (D18)

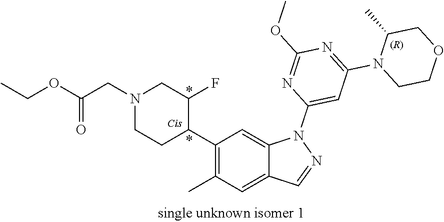

To a solution of cis-(3R)-4-(6-(6-(3-fluoropiperidin-4-yl)-5-methyl-1H-indazol-1-yl)-2-methoxypyrimidin-4-yl)-3-methylmorpholine hydrochloride(D17, 75 mg, 0.17 mmol)) in DMF (2 mL) were added Et₃N (52 mg, 0.51 mmol) and ethyl 2-bromoacetate (57 mg, 0.34 mmol) in ice bath. The reaction mixture was stirred at RT for 2 hours, diluted with water (20 mL) and extracted with EtOAc (30 mL×3). The combined organic layers were washed with brine (100 mL×2), dried over anhydrous Na₂SO₄ and concentrated to give the title product as a white solid (90 mg).

LC-MS [mobile phase: 70% water (0.1% FA) and 30% CH₃CN (0.1% FA) to 5% water (0.1% FA) and 95% CH₃CN (0.1% FA) in 2.0 min]: Rt=1.54 min; MS Calcd.: 526.6, MS Found: 527.3 [M+H]⁺.

### Description 19

### tert-butyl cis-3-fluoro-4-(1-(2-methoxy-6-((R)-3-methylmorpholino)pyrimidin-4-yl)-5-methyl-1H-indazol-6-yl)piperidine-1-carboxylate (D19)

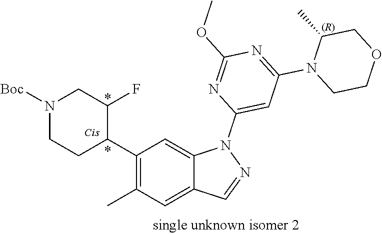

The title compounds were prepared by a procedure similar to those described for D16 starting from cis-3-fluoro-4-(5-methyl-1H-indazol-6-yl)piperidine-1-carboxylate (D10), (R)-4-(6-iodo-2-methylpyrimidin-4-yl)-3-methylmorpholine in toluene/THF, N1,N2-dimethylethane-1,2-diamine, CuI and K₃PO₄ at 90° C.

LC-MS [mobile phase: 40% water (0.1% FA) and 60% CH₃CN (0.1% FA) to 5% water (0.1% FA) and 95% CH₃CN (0.1% FA) in 9.0 min]: Rt=4.48 min; MS Calcd.: 540.6, MS Found: 541.3 [M+H]⁺.

### Description 20

### cis-(3R)-4-(6-(6-(3-fluoropiperidin-4-yl)-5-methyl-1H-indazol-1-yl)-2-methoxypyri-midin-4-yl)-3-methylmorpholine hydrochloride (D20)

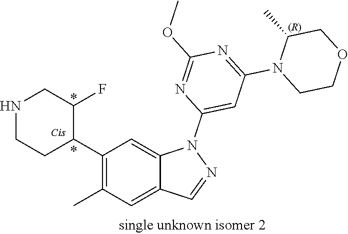

The title compounds were prepared by a procedure similar to those described for D17 starting from tert-butyl cis-3-fluoro-4-(1-(2-methoxy-6-((R)-3-methylmorpholino) pyrimidin-4-yl)-5-methyl-1H-indazol-6-yl)piperidine-1-carboxylate (D19).

LC-MS [mobile phase: 90% water (0.1% FA) and 10% CH₃CN (0.1% FA) to 5% water (0.1% FA) and 95% CH₃CN (0.1% FA) in 9.0 min]: Rt=4.87 min; MS Calcd.: 440.5, MS Found: 441.3 [M+H]⁺.

### Description 21

### ethyl cis-2-(3-fluoro-4-(1-(2-methoxy-6-((R)-3-methylmorpholino)pyrimidin-4-yl)-5-methyl-1H-indazol-6-yl)piperidin-1-yl)acetate (D21)

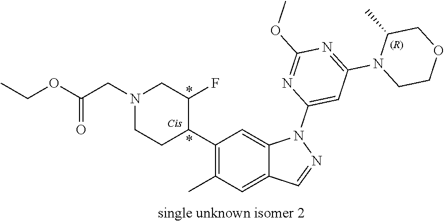

The title compounds were prepared by a procedure similar to those described for D18 starting from cis-(3R)-4-(6-(6-(3-fluoropiperidin-4-yl)-5-methyl-1H-indazol-1-yl)-2-methoxypyrimidin-4-yl)-3-methylmorpholine hydrochloride and ethyl 2-bromoacetate (D20).

LC-MS [mobile phase: 70% water (0.1% FA) and 30% CH₃CN (0.1% FA) to 5% water (0.1% FA) and 95% CH₃CN (0.1% FA) in 2.0 min]: Rt=1.54 min; MS Calcd.: 526.6, MS Found: 527.3 [M+H]⁺.

### Descriptions 22-50

Below compounds were prepared by a procedure similar to those described for D13, D16, D17 and D18.

| Entry | Structure | Solvent/base | Characterization |
|---|---|---|---|
| D 22 | 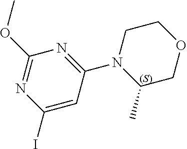 | i-PrOH & THF/DIPEA | LC-MS: mobile phase: mobile phase: from 60% water (0.1% FA) and 40% CH₃CN (0.1% FA) to 5% water (0.1% FA) and 95% CH₃CN (0.1% FA) in 2.0 min, Rt 1.43 min; MS Calcd.: 335.0, MS Found: 336.0 [M + H]⁺. |
| D 23 | 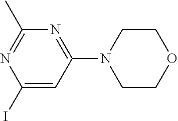 | i-PrOH & THF/NEt₃ | LC-MS: mobile phase: mobile phase: from 70% water (0.1% FA) and 30% CH₃CN (0.1% FA) to 5% water (0.1% FA) and 95% CH₃CN (0.1% FA) in 2.0 min, Rt 0.45 min; MS Calcd.: 305, MS Found: 306.2 [M + H]⁺. |
| D 24 | 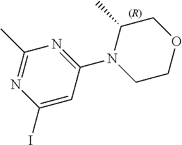 | i-PrOH/ DIEDA | |
| D 25 | 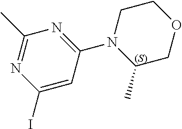 | i-PrOH & THF/DIPEA | LC-MS: mobile phase: mobile phase: from 60% water (0.1% FA) and 40% CH₃CN (0.1% FA) to 5% water (0.1% FA) and 95% CH₃CN (0.1% FA) in 2.0 min, Rt = 0.99 min; MS Calcd.: 319.0, MS Found: 320.2 [M + H]⁺. |
| D 26 | 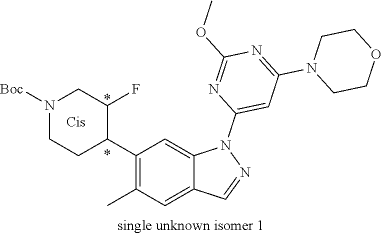 [SMILES: COc1nc(N2CCOCC2)cc(-n2ncc3cc(C)c(C4CCN(C(=O)OC(C)(C)C)CC4F)cc32)n1] | Toluene & THF/DMEDA. | LC-MS: mobile phase: mobile phase: from 90% water (0.1% FA) and 10% CH₃CN (0.1% FA) to 5% water (0.1% FA) and 95% CH₃CN (0.1% FA) in 2.0 min, Rt = 1.81 min; MS Calcd.: 526.3, MS Found: 527.4 [M + H]⁺. |
| D 27 | 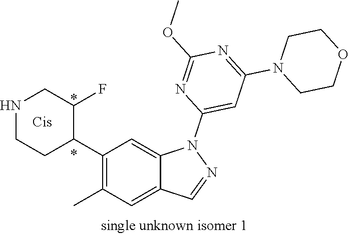 | EtOAc | LC-MS: mobile phase: mobile phase: from 90% water (0.1% FA) and 10% CH₃CN (0.1% FA) to 5% water (0.1% FA) and 95% CH₃CN (0.1% FA) in 2.0 min, Rt = 1.16 min; MS Calcd.: 438.2, MS Found: 439.4 [M + H]⁺. |
| D 28 | 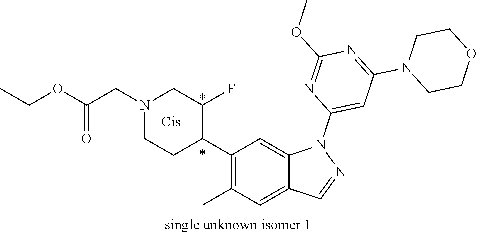 [SMILES: CCOC(=O)CN1CCC(c2cc3c(cnn3-c3cc(N4CCOCC4)nc(OC)n3)cc2C)C(F)C1] | DMF/NEt₃ | LC-MS: mobile phase: mobile phase: from 90% water (0.1% FA) and 10% CH₃CN (0.1% FA) to 5% water (0.1% FA) and 95% CH₃CN (0.1% FA) in 2.0 min, Rt = 1.56 min; MS Calcd.: 512.3, MS Found: 513.4 [M + H]⁺. |
| D 29 | 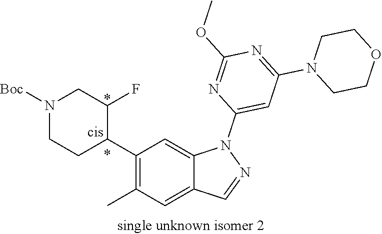 [SMILES: COc1nc(N2CCOCC2)cc(-n2ncc3cc(C)c(C4CCN(C(=O)OC(C)(C)C)CC4F)cc32)n1] | | LC-MS: mobile phase: mobile phase: from 90% water (0.1% FA) and 10% CH₃CN (0.1% FA) to 5% water (0.1% FA) and 95% CH₃CN (0.1% FA) in 9.0 min, Rt = 6.93 min; MS Calcd.: 526.3, MS Found: 527.3 [M + H]⁺. |
| D 30 | 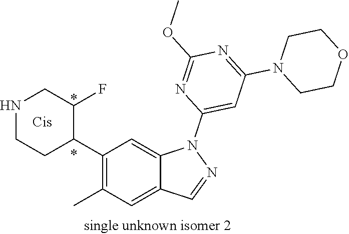 | | LC-MS: mobile phase: mobile phase: from 90% water (0.1% FA) and 10% CH₃CN (0.1% FA) to 5% water (0.1% FA) and 95% CH₃CN (0.1% FA) in 2.0 min, Rt = 1.16 min; MS Calcd.: 438.2, MS Found: 439.4 [M + H]⁺. |
| D 31 | 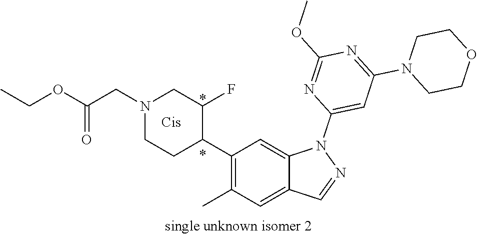 [SMILES: CCOC(=O)CN1CCC(c2cc3c(cnn3-c3cc(N4CCOCC4)nc(OC)n3)cc2C)C(F)C1] | DMF/NEt₃ | LC-MS: mobile phase: mobile phase: from 90% water (0.1% FA) and 10% CH₃CN (0.1% FA) to 5% water (0.1% FA) and 95% CH₃CN (0.1% FA) in 9.0 min, Rt = 4.82 min; MS Calcd.: 512.3, MS Found: 513.3 [M + H]⁺. |
| D 32 | 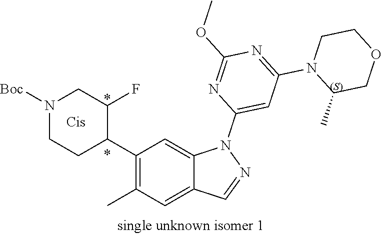 | toluene/THF/ DMEDA | LC-MS: mobile phase: mobile phase: from 90% water (0.1% FA) and 10% CH₃CN (0.1% FA) to 5% water (0.1% FA) and 95% CH₃CN (0.1% FA) in 9.0 min, Rt = 7.02 min; MS Calcd.: 540.3, MS Found: 541.3 [M + H]⁺. |
| D 33 | 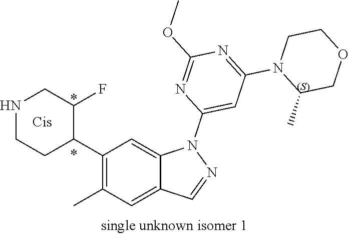 | EtOAC | LC-MS: mobile phase: mobile phase: from 90% water (0.1% FA) and 10% CH₃CN (0.1% FA) to 5% water (0.1% FA) and 95% CH₃CN (0.1% FA) in 9.0 min, Rt = 4.92 min; MS Calcd.: 440.2, MS Found: 441.3 [M + H]⁺. |
| D 34 | 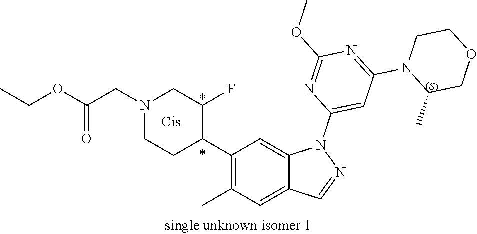 | Et₃N/DMF | LC-MS: mobile phase: mobile phase: from 90% water (0.1% FA) and 10% CH₃CN (0.1% FA) to 5% water (0.1% FA) and 95% CH₃CN (0.1% FA) in 2.0 min, Rt = 1.61 min; MS Calcd.: 526.3, MS Found: 527.3 [M + H]⁺. |
| D 35 | 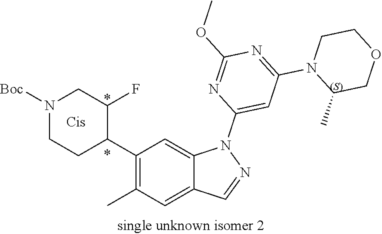 | toluene & THF/DMEDA | LC-MS: mobile phase: mobile phase: from 90% water (0.1% FA) and 10% CH₃CN (0.1% FA) to 5% water (0.1% FA) and 95% CH₃CN (0.1% FA) in 9.0 min, Rt = 4.50 min; MS Calcd.: 540.3, MS Found: 541.3 [M + H]⁺. |
| D 36 | 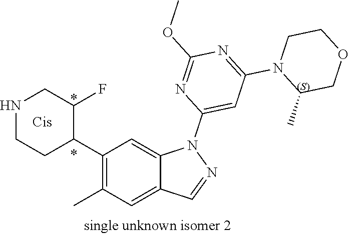 | EtOAc | LC-MS: mobile phase: mobile phase: from 90% water (0.1% FA) and 10% CH₃CN (0.1% FA) to 5% water (0.1% FA) and 95% CH₃CN (0.1% FA) in 9.0 min, Rt = 4.91 min; MS Calcd.: 440.2, MS Found: 441.3 [M + H]⁺. |
| D 37 | 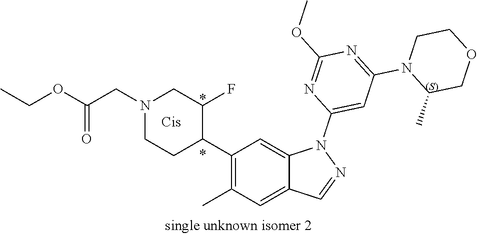 | NEt₃/DMF | LC-MS: mobile phase: mobile phase: from 90% water (0.1% FA) and 10% CH₃CN (0.1% FA) to 5% water (0.1% FA) and 95% CH₃CN (0.1% FA) in 2.0 min, Rt = 1.61 min; MS Calcd.: 526.3, MS Found: 527.3 [M + H]⁺. |
| D 38 | 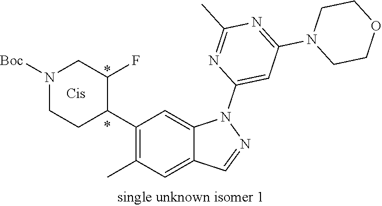 [SMILES: Cc1nc(N2CCOCC2)cc(-n2ncc3cc(C)c(C4CCN(C(=O)OC(C)(C)C)CC4F)cc32)n1] | toluene/DMEDA | LC-MS: mobile phase: mobile phase: from 50% water (0.1% FA) and 50% CH₃CN (0.1% FA) to 5% water (0.1% FA) and 95% CH₃CN (0.1% FA) in 3.0 min, Rt 1.57 min; MS Calcd.: 510, MS Found: 511.4[M + H]+. |
| D 39 | 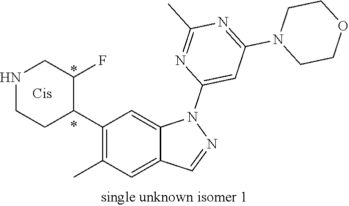 [SMILES: Cc1nc(N2CCOCC2)cc(-n2ncc3cc(C)c(C4CCNCC4F)cc32)n1] | EtOAc | |
| D 40 | 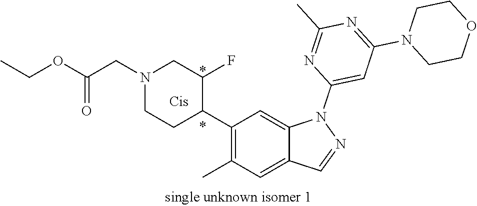 [SMILES: CCOC(=O)CN1CCC(c2cc3c(cnn3-c3cc(N4CCOCC4)nc(C)n3)cc2C)C(F)C1] | NEt₃/DMf | LC-MS: mobile phase: mobile phase: from 90% water (0.1% FA) and 10% CH₃CN (0.1% FA) to 5% water (0.1% FA) and 95% CH₃CN (0.1% FA) in 9.0 min, Rt = 6.15 min; MS Calcd.: 496, MS Found: 497.3 [M + H]⁺. |
| D 41 | 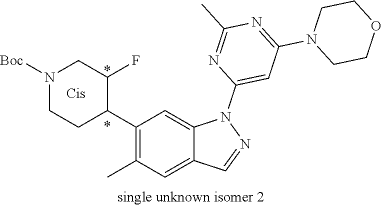 [SMILES: Cc1nc(N2CCOCC2)cc(-n2ncc3cc(C)c(C4CCN(C(=O)OC(C)(C)C)CC4F)cc32)n1] | Toluene/ DMEDA | LC-MS: mobile phase: mobile phase: from 80% water (0.1% FA) and 20% CH₃CN (0.1% FA) to 5% water (0.1% FA) and 95% CH₃CN (0.1% FA) in 9.0 min, Rt = 7.66 min; MS Calcd.: 510, MS Found: 511.3 [M + H]⁺. |
| D 42 | 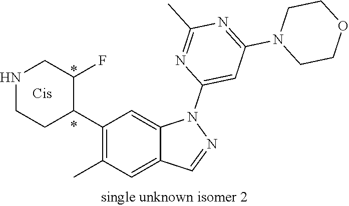 [SMILES: Cc1nc(N2CCOCC2)cc(-n2ncc3cc(C)c(C4CCNCC4F)cc32)n1] | EtOAc | |
| D 43 | 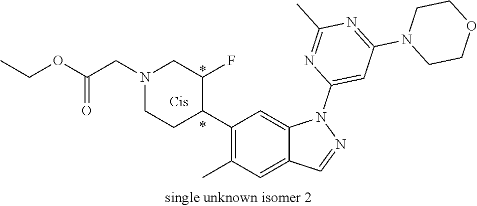 [SMILES: CCOC(=O)CN1CCC(c2cc3c(cnn3-c3cc(N4CCOCC4)nc(C)n3)cc2C)C(F)C1] | NEt₃/DMF | LC-MS: mobile phase: mobile phase: from 90% water (0.1% FA) and 10% CH₃CN (0.1% FA) to 5% water (0.1% FA) and 95% CH₃CN (0.1% FA) in 9.0 min, Rt 6.15 min; MS Calcd.: 496, MS Found: 497.3 [M + H]⁺. |
| D 44 | 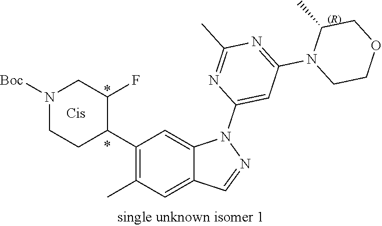 | Toluene & THF/N¹,N²- dimethylethane- 1,2,-diamine | LC-MS: mobile phase: 50% water (0.1% FA) and 50% CH₃CN (0.1% FA) to 5% water (0.1% FA) and 95% CH₃CN (0.1% FA) in 10.0 min, Rt = 7.41 min; MS Calcd.:524.6, MS Found: 525.4 [M + H]⁺. |
| D 45 | 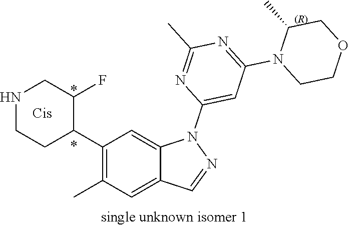 | EtOAc | LC-MS: mobile phase: 90% water (0.1% FA) and 10% CH₃CN (0.1% FA) to 5% water (0.1% FA) and 95% CH₃CN (0.1% FA) in 10.0 min, Rt = 4.70 min; MS Calcd.:424.5, MS Found: 425.3 [M + H]⁺. |
| D 46 | 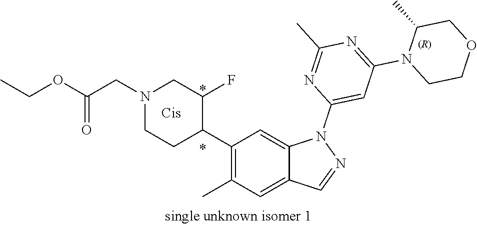 | NEt₃/DMF | LC-MS: mobile phase: 70% water (0.1% FA) and 30% CH₃CN (0.1% FA) to 5% water (0.1% FA) and 95% CH₃CN (0.1% FA) in 2.0 min, Rt = 1.54 min; MS Calcd.:510.6, MS Found: 511.3 [M + H]⁺. |
| D 47 | 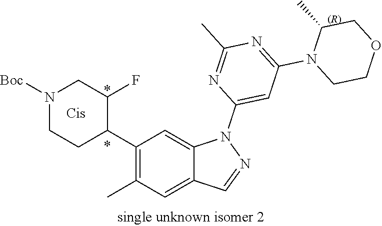 | toluene & THF/N¹,N²- dimethylethane- 1,2,-diamine | LC-MS: mobile phase: 40% water (0.1% FA) and 60% CH₃CN (0.1% FA) to 5% water (0.1% FA) and 95% CH₃CN (0.1% FA) in 9.0 min, Rt = 4.89 min; MS Calcd.:524.6, MS Found: 525.4 [M + H]⁺. |
| D 48 | 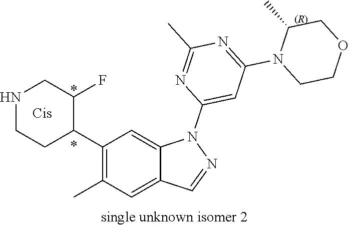 | EtOAc | LC-MS: mobile phase: 90% water (0.1% FA) and 10% CH₃CN (0.1% FA) to 5% water (0.1% FA) and 95% CH₃CN (0.1% FA) in 9.0 min, Rt = 4.76 min; MS Calcd.:424.5, MS Found: 425.3 [M + H]⁺. |
| D 49 | 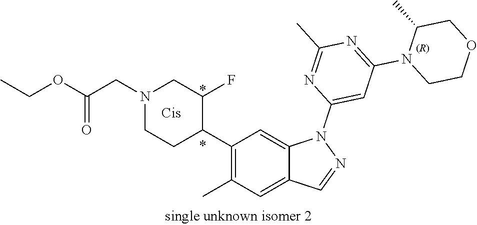 | DMF/Et₃N | LC-MS: mobile phase: 70% water (0.1% FA) and 30% CH₃CN (0.1% FA) to 5% water (0.1% FA) and 95% CH₃CN (0.1% FA) in 2.0 min, Rt = 1.54 min; MS Calcd.:510.6, MS Found: 511.3 [M + H]⁺. |

### Description 50

### tert-butyl 4-(5-methyl-1-(tetrahydro-2H-pyran-2-yl)-1H-indazol-6-yl)piperidine-1-carboxylate (D50)

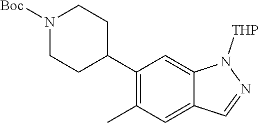

To a solution of tert-butyl 4-(5-methyl-1-(tetrahydro-2H-pyran-2-yl)-1H-indazol-6-yl)-5,6-dihydropyridine-1(2H)-carboxylate (80 g, crude) in MeOH (2 L) under H₂ was added Pd/C (10 g, 12%/W). The reaction mixture was degassed for 3 times and stirred at r.t for 2 d. The mixture was filtered and the filtrate was concentrated to give the crude product as a white solid. (65.8 g)

LC-MS [mobile phase: mobile phase: from 30% water (0.1% FA) and 70% CH₃CN (0.1% FA) to 5% water (0.1% FA) and 95% CH₃CN (0.1% FA) in 2.0 min]: Rt=0.63 min; MS Calcd.: 399.2, MS Found: 400.5 [M+H]⁺.

### Description 51

### 5-methyl-6-(piperidin-4-yl)-1H-indazole (D51)

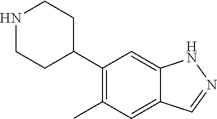 [SMILES: Cc1cc2cn[nH]c2cc1C1CCNCC1]

To a solution of tert-butyl 4-(5-methyl-1-(tetrahyd-ro-2H-pyran-2-yl)-1H-indazol-6-yl)piperidine-1-carboxylate (55.4 g, 139 mmol) in MeOH (150 mL) was added HCl/MeOH (5 M, 200 mL). The reaction mixture was stirred at rt overnight, then concentrated, treated with a solution of Na₂CO₃ and basified with a solution of NaOH to pH >12. The mixture was filtered to give the desired product as a white solid. (29.3 g, yield=98%)

LC-MS [mobile phase: mobile phase: from 90% water (0.1% FA) and 10% CH₃CN (0.1% FA) to 5% water (0.1% FA) and 95% CH₃CN (0.1% FA) in 2.0 min]: Rt=0.85 min; MS Calcd.: 215, MS Found: 216 [M+H]⁺.

### Description 52

### tert-butyl 4-(5-methyl-1H-indazol-6-yl)piperidine-1-carboxylate (D52)

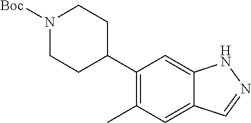 [SMILES: Cc1cc2cn[nH]c2cc1C1CCN(C(=O)OC(C)(C)C)CC1]

To a stirred solution of 5-methyl-6-(piperidin-4-yl)-1H-indazole (1.00 g, 4.64 mmol) and Et₃N (930 mg, 9.20 mmol) in CH₂Cl₂ (80 mL) was added Boc₂O (1.00 g, 4.60 mmol). The reaction mixture was stirred at room temperature for 3 h. LC-MS showed the reaction was completed. The reaction mixture was concentrated to dryness. The residue was purified by silica gel chromatography eluted with PE:EtOAc=3:1 to afford the desired product as a white solid (900 mg, yield: 61%).

¹H NMR (400 MHz, DMSO-d₅) δ 12.77 (s, 1H), 7.89 (s, 1H), 7.50 (s, 1H), 7.28 (s, 1H), 4.12-4.07 (m, 2H), 3.17 (s, 1H), 2.94-2.84 (m, 2H), 2.40 (s, 3H), 1.77 (d, J=12.0 Hz, 2H), 1.55-1.47 (m, 2H), 1.43 (s, 9H).

### Description 53

### 1-(4-(6-iodo-2-methoxypyrimidin-4-yl)morpholin-2-yl)ethanone (D53)

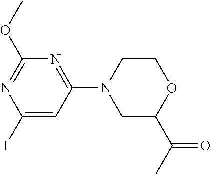 [SMILES: COc1nc(I)cc(N2CCOC(C(C)=O)C2)n1]

To a solution of 4,6-diiodo-2-methoxypyrimidine (869 mg, 2.40 mmol) and 1-(morpholin-2-yl)ethanone (400 mg, 2.40 mmol) in THF/EtOH=1/1 (30 mL) was added DIEA (1.24 g, 9.60 mmol) at rt. The reaction mixture was stirred at rt for 16 h. TLC (PE:EtOAc=5:1) showed reaction was completed. The reaction mixture was concentrated to dryness and the residue was purified by silica gel chromatography eluted with PE:EtOAc=10:1 to afford the title product as a white solid (650 mg, yield: 74%).

LC-MS [mobile phase: from 50% water (0.1% FA) and 50% CH₃CN (0.1% FA) to 5% water (0.1% FA) and 95% CH₃CN (0.1% FA) in 2.6 min]: Rt=1.13 min; MS Calcd: 363.0, MS Found: 364.0 [M+H]⁺.

### Description 54

### tert-butyl 4-(1-(6-(2-acetylmorpholine)-2-methoxypyrimidin-4-yl)-5-methyl-1H-indazol-6-yl)piperidine-1-carboxylate (D54)

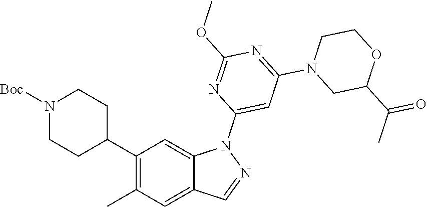 [SMILES: COc1nc(N2CCOC(C(C)=O)C2)cc(-n2ncc3cc(C)c(C4CCN(C(=O)OC(C)(C)C)CC4)cc32)n1]

To a stirred solution of 1-(4-(6-iodo-2-methoxypyrimidin-4-yl)morpholin-2-yl)ethanone (576 mg, 1.60 mmol) and tert-butyl 4-(5-methyl-1H-indazol-6-yl)piperidine-1-carboxylate (500 mg, 1.60 mmol) in toluene (30 mL) were added CuI (453 mg, 2.03 mmol), K₃PO₄ (672 mg, 3.20 mmol) and N,N′-dimethylethylenediamine (281 mg, 3.20 mmol). The reaction mixture was stirred at 100° C. for 5 h. LC-MS showed reaction was completed. The reaction mixture was concentrated and the residue was purified by silica gel chromatography eluted with PE:EtOAc=3:1 to give the title product as a white solid (550 mg, yield: 63%).

LC-MS [mobile phase: from 50% water (0.1% FA) and 50% CH₃CN (0.1% FA) to 5% water (0.1% FA) and 95% CH₃CN (0.1% FA) in 2.6 min]: Rt=2.01 min; MS Calcd: 550.3, MS Found: 551.4 [M+H]⁺.

### Description 55

### 1-(4-(2-methoxy-6-(5-methyl-6-(piperidin-4-yl)-1H-indazol-1-yl)pyrimidin-4-yl)morpholin-2-yl)ethanone (D55)

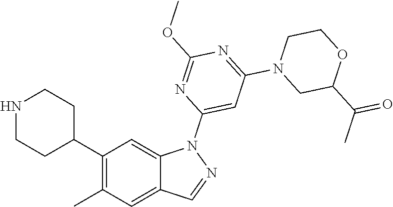 [SMILES: COc1nc(N2CCOC(C(C)=O)C2)cc(-n2ncc3cc(C)c(C4CCNCC4)cc32)n1]

To a solution of tert-butyl 4-(1-(6-(2-acetylmorpholino)-2-methoxypyrimidin-4-yl)-5-methyl-1H-indazol-6-yl)piperidine-1-carboxylate (540 mg, 0.98 mmol) in CH₂Cl₂ (50 mL) was added 2,2,2-trifluoroacetic acid (5 mL). The reaction mixture was stirred at rt for 16 h. LC-MS showed the reaction was completed. The reaction mixture was concentrated and the residue was diluted with CH₂Cl₂ (20 mL) and NH₃.H₂O (10 mL). The aqueous layer was extracted with CH₂Cl₂ (2×20 mL) and the combined organic layers were washed with brine, dried over anhydrous Na₂SO₄ and filtered. The filtrate was concentrated to dryness to give the target product as a white solid (406 mg, yield: 92%).

LC-MS [mobile phase: from 50% water (0.1% FA) and 50% CH₃CN (0.1% FA) to 5% water (0.1% FA) and 95% CH₃CN (0.1% FA) in 2.6 min]: Rt=0.79 min; MS Calcd: 450.2, MS Found: 451.2 [M+H]⁺.

### Description 56

### 1-(4-(6-(6-(1-(2-fluoroethyl)piperidin-4-yl)-5-methyl-1H-indazol-1-yl)-2-methoxypyrimidin-4-yl)morpholin-2-yl)ethanone (D56)

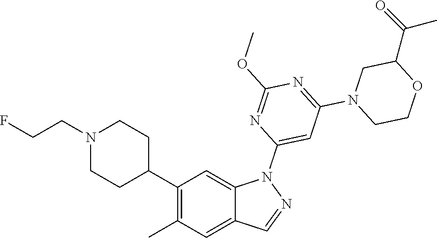 [SMILES: COc1nc(N2CCOC(C(C)=O)C2)cc(-n2ncc3cc(C)c(C4CCN(CCF)CC4)cc32)n1]

To a solution of 1-(4-(2-methoxy-6-(5-methyl-6-(piperidin-4-yl)-1H-indazol-1-yl)pyrimidin-4-yl)morpholin-2-yl)ethanone (380 mg, 0.840 mmol), 1-fluoro-2-iodoethane (176 mg, 1.01 mmol) in DMF (20 mL) was added Cs₂CO₃ (326 mg, 1.69 mmol). The reaction mixture was stirred at rt overnight. LC-MS showed reaction was completed. The reaction mixture was diluted with CH₂Cl₂ (20 mL) and H₂O (20 mL). The aqueous layer was extracted with CH₂Cl₂ (2×50 mL) and the combined organic layers were washed with brine, dried over anhydrous Na₂SO₄ and filtered. The filtrate was concentrated to dryness. The residue was purified by silica gel chromatography eluted with EtOAc to give the title product as a white solid (235 mg, yield: 56%).

LC-MS [mobile phase: from 50% water (0.1% FA) and 50% CH₃CN (0.1% FA) to 5% water (0.1% FA) and 95% CH₃CN (0.1% FA) in 2.6 min]: Rt=0.80 min; MS Calcd: 496.6, MS Found: 497.4 [M+H]⁺.

### Description 57

### 4,6-Diiodo-2-methylpyrimidine (D57)

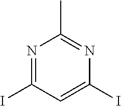

To a solution of NaI (11.9 g, 79.7 mmol) in HI (55%, 50 mL) was added 4,6-dichloro-2-methylpyrimidine (10.0 g, 61.3 mmol) in portions. The reaction mixture was heated to 40° C. and stirred for 1 hour, then filtered, washed with water then methanol (50 mL) and filtered. The filtered cake was dried to give the title compound (9.0 g, yield 42%) as a white solid. ¹H NMR (400 MHz, CDCl₃): δ 8.07 (s, 1H), 2.67 (s, 3H).

LCMS (mobile phase: 5-95% acetonitrile in 2.5 min): Rt=1.59 min, MS Calcd: 346; MS Found: 347 [M+H]⁺.

### Description 58

### 6-((3S,4R)-3-fluoro-1-(2-fluoroethyl)piperidin-4-yl)-5-methyl-1H-indazole (D58)

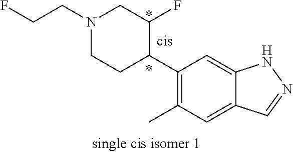 [SMILES: Cc1cc2cn[nH]c2cc1C1CCN(CCF)CC1F]

To a suspension of 6-((3S,4R)-3-fluoropiperidin-4-yl)-5-methyl-1H-indazole (D11) (80 mg, 0.34 mmol) and 1-bromo-2-fluoroethane (52.0 mg, 0.41 mmol) in DMF (2 mL) was added K₂CO₃ (141 mg, 1.02 mmol). The reaction mixture was stirred at 25° C. for one day, quenched with water and extracted with EtOAc for 3 times. The organic phase was combined, dried, filtered and concentrated. The purification via silico gel column(EtOAc DCM:MeOH=20:1) afforded the title product (40 mg, yield 41.8%) as a white solid.

### Description 59

### 6-((3S,4R)-3-fluoro-1-(2-fluoroethyl)piperidin-4-yl)-5-methyl-1H-indazole (D59)

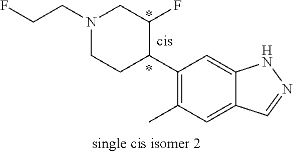 [SMILES: Cc1cc2cn[nH]c2cc1C1CCN(CCF)CC1F]

The title compound was prepared by a procedure similar to those described for D58 starting from a suspension of 6-((3R,4S)-3-fluoropiperidin-4-yl)-5-methyl-1H-indazole (D12), 1-bromo-2-fluoroethane, K₂CO₃ in DMF at 25° C.

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=0.29 min; MS Calcd.: 279.1, MS Found: 280.2 [M+H]⁺.

### Description 60

### 1-(6-Iodo-2-methylpyrimidin-4-yl)azetidin-3-ol (D60)

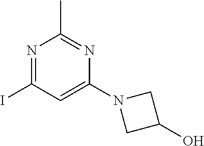

The title compound was prepared by a procedure similar to those described for D14 starting from a suspension of 4,6-diiodo-2-methylpyrimidine, azetidin-3-ol hydrochloride and TEA in i-PrOH.

¹H NMR (400 MHz, DMSO-d₆): δ 6.69 (s, 1H), 5.79 (d, J=6.4 Hz, 1H), 4.59-4.52 (m, 1H), 4.22-4.18 (m, 2H), 3.72 (dd, J=9.6, 4.4 Hz, 2H), 2.29 (s, 3H).

LCMS: (mobile phase: 5-95% Acetonitrile in 2.5 min), Rt=1.18 min, MS Calcd: 291; MS Found: 292 [M+H]⁺.

### Description 61

### 4-Iodo-2-methyl-6-(3-((tetrahydro-2H-pyran-2-yl)oxy)azetidin-1-yl)pyrimidine (D61)

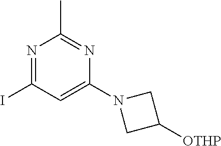

To a suspension of 1-(6-iodo-2-methylpyrimidin-4-yl)azetidin-3-ol (1.20 g, 4.12 mmol) in dry DCM (20 mL) was added DHP (1.38 g, 16.4 mmol) and TsOH (280 mg, 1.64 mmol) at rt. The resulting mixture was heated to reflux and stirred for 20 hrs. The reaction mixture was diluted with DCM to 100 mL and then washed with Na₂CO₃ (sat., 50 mL) and brine, dried over MgSO₄ and concentrated. The crude was purified by column chromatography (PE:EtOAc=5:1) to give the title compound (1.5 g, yield 97%) as a colorless oil.

¹H NMR (300 MHz, CDCl₃): δ 6.48 (s, 1H), 4.70-4.60 (m, 2H), 4.33-4.18 (m, 2H), 4.08-3.92 (m, 2H), 3.90-3.80 (m, 1H), 3.57-3.48 (m, 1H), 2.45 (s, 3H), 1.89-1.69 (m, 2H), 1.64-1.49 (m, 4H).

LCMS (mobile phase: 5-95% Acetonitrile in 2.5 min): Rt=1.59 min, MS Calcd: 375; MS Found: 376 [M+H]⁺.

### Description 62

### 6-((3S,4R)-3-fluoro-1-(2-fluoroethyl)piperidin-4-yl)-5-methyl-1-(2-methyl-6-(3-((tetrahydro-2H-pyran-2-yl)oxy)azetidin-1-yl)pyrimidin-4-yl)-1H-indazole (D62)

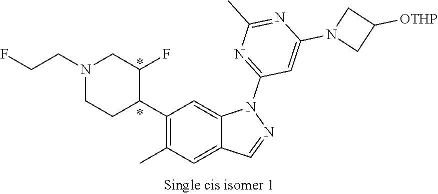

The title compounds were prepared by a procedure similar to those described for D16 starting from a suspension of 6-(3-fluoro-1-(2-fluoroethyl)piperidin-4-yl)-5-methyl-1-(2-methyl-6-(3-((tetrahydro-2H-pyran-2-yl)oxy)azetidin-1-yl)pyrimidin-4-yl)-1H-indazole (D58), 4-iodo-2-methyl-6-(3-((tetrahydro-2H-pyran-2-yl)oxy)azetidin-1-yl)pyrim-idine, CuI and K₃PO₄ in dry toluene and N,N-dimethyl-1,2-ethanediamine.

### Description 63

### 6-((3S,4R)-3-fluoro-1-(2-fluoroethyl)piperidin-4-yl)-5-methyl-1-(2-methyl-6-(3-((tetrahydro-2H-pyran-2-yl)oxy)azetidin-1-yl)pyrimidin-4-yl)-1H-indazole (D63)

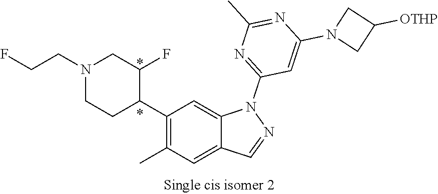

The title compounds were prepared by a procedure similar to those described for D16 starting from a suspension of 6-((3R,4S)-3-fluoro-1-(2-fluoroethyl)piperidin-4-yl)-5-methyl-1H-indazole (D59), 4-iodo-2-methyl-6-(3-((tetrahydro-2H-pyran-2-yl)oxy)azetidin-1-yl)pyrimidine, CuI and K₃PO₄ in dry toluene and N,N-dimethyl-1,2-ethanediamine.

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=1.18 min; MS Calcd.: 526.3, MS Found: 527.3 [M+H]⁺.

### Description 64

### (R)-tert-butyl 4-(5-methyl-1-(2-methyl-6-(3-methylmorpholino)pyrimidin-4-yl)-1H-indazol-6-yl)piperidine-1-carboxylate (D64)

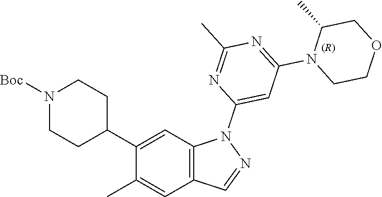

The title compound was prepared by a procedure similar to those described for D16 starting from a mixture of tert-butyl 4-(5-methyl-1H-indazol-6-yl)piperidine-1-carboxylate and (R)-4-(6-iodo-2-methylpyrimidin-4-yl)-3-methylmorpholine in toluene/THF, N¹,N²-dimethylethane-1,2-diamine, CuI and K₃PO₄.

LC-MS [mobile phase: 40% water (0.1% FA) and 60% CH₃CN (0.1% FA) in 10.0 min]: Rt=5.99 min; MS Calcd.: 506.6, MS Found: 507.4 [M+H]⁺.

### Description 65

### (R)-3-methyl-4-(2-methyl-6-(5-methyl-6-(piperidin-4-yl)-1H-indazol-1-yl)pyrimidin-4-yl)morpholine (D65)

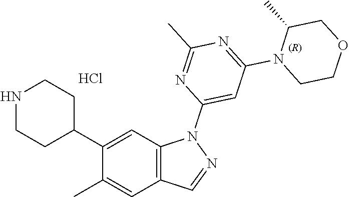

The title compound was prepared by a procedure similar to those described for D17 starting from (R)-tert-butyl 4-(5-methyl-1-(2-methyl-6-(3-methylmorpholino)pyrimidin-4-yl)-1H-indazol-6-yl)piperidine-1-carboxylate in EtOAc and HCl.EtOAc.

### Description 66

### (R)-ethyl 2-(4-(5-methyl-1-(2-methyl-6-(3-methylmorpholino)pyrimidin-4-yl)-1H-indazol-6-yl)piperidin-1-yl)acetate (D66)

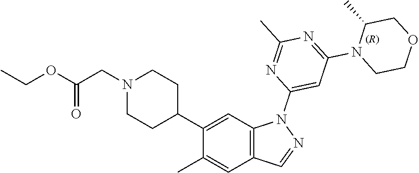

The title compound was prepared by a procedure similar to those described for D18 starting from a solution of (R)-3-methyl-4-(2-methyl-6-(5-methyl-6-(piperidin-4-yl)-1H-indazol-1-yl)pyrimidin-4-yl)morpholine in DMF, Et₃N and ethyl 2-bromoacetate.

LC-MS [mobile phase: 70% water (0.1% FA) and 30% CH₃CN (0.1% FA) in 2.0 min]: Rt=1.17 min; MS Calcd.: 492.6, MS Found: 493.4 [M+H]⁺.

### Description 67

### (R)-tert-butyl 4-(1-(2-methoxy-6-(3-methylmorpholino)pyrimidin-4-yl)-5-methyl-1H-indazol-6-yl)piperidine-1-carboxylate (D67)

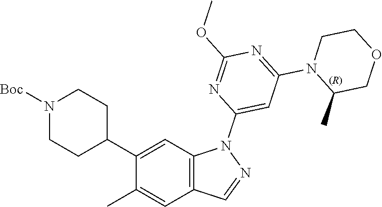

The title compound was prepared by a procedure similar to those described for D16 starting from a mixture of tert-butyl 4-(5-methyl-1H-indazol-6-yl)piperidine-1-carboxylate, (R)-4-(6-iodo-2-methoxypyrimidin-4-yl)-3-methylmorpholine, CuI (20 mg), K₃PO₄ in toluene/THF and DMEDA

¹H NMR (400 MHz, CDCl₃): δ 8.72 (s, 1H), 8.06 (s, 1H), 7.51 (s, 1H), 6.80 (s, 1H), 4.46 (br, 1H), 4.29 (br, 2H), 4.11-4.10 (m, 1H), 4.11 (s, 3H), 4.03-3.99 (m, 1H), 3.78-3.73 (m, 2H), 3.61-3.53 (m, 1H), 3.37-3.30 (m, 1H), 3.02-2.85 (m, 3H), 2.47 (s, 3H), 1.86 (m, 2H), 1.71-1.65 (m, 2H), 1.50 (s, 9H), 1.34 (d, J=6.8 Hz, 3H).

### Description 68

### Synthesis of (R)-4-(2-methoxy-6-(5-methyl-6-(piperidin-4-yl)-1H-indazol-1-yl)pyrimidin-4-yl)-3-methylmorpholine hydrochloride (D68)

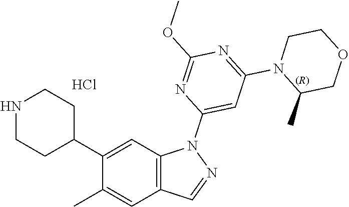

The title compounds were prepared by a procedure similar to those described for D17 starting from a solution of (R)-tert-butyl 4-(1-(2-methoxy-6-(3-methylmorpholino)pyrimidin-4-yl)-5-methyl-1H-indazol-6-yl)piperidine-1-carboxylate in HCl/EtOAc.

### Description 69

### (R)-ethyl 2-(4-(1-(2-methoxy-6-(3-methylmorpholino)pyrimidin-4-yl)-5-methyl-1H-indazol-6-yl)piperidin-1-yl)acetate (D69)

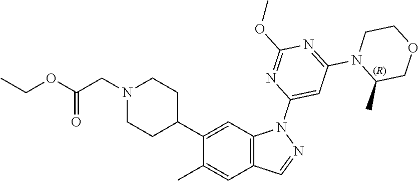

The title compound was prepared by a procedure similar to those described for D18 starting from ethyl 2-bromoacetate and a solution of (R)-4-(2-methoxy-6-(5-methyl-6-(piperidin-4-yl)-1H-indazol-1-yl)pyrimidin-4-yl)-3-methylmorpholine hydrochloride and Et₃N in DMF.

### Description 70

### (S)-tert-butyl 4-(1-(2-methoxy-6-(3-methylmorpholino)pyrimidin-4-yl)-5-methyl-1H-indazol-6-yl)piperidine-1-carboxylate (D70)

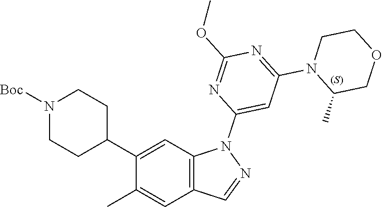

The title compound was prepared by a procedure similar to those described for D16 starting from a suspension of tert-butyl 4-(5-methyl-1H-indazol-6-yl)piperidine-1-carboxylate, (S)-4-(6-iodo-2-methoxypyrimidin-4-yl)-3-methylmorpholine, CuI and K₃PO₄ in toluene/THF and DMEDA.

LC-MS[mobile phase: from 90% water (0.1% FA) and 10% CH₃CN (0.1% FA) to 5% water (0.1% FA) and 95% CH₃CN (0.1% FA) in 2.0 min]: Rt=1.07 min; MS Calcd.: 522.3, MS Found: 523.4 [M+H]⁺.

### Description 71

### (S)-4-(2-methoxy-6-(5-methyl-6-(piperidin-4-yl)-1H-indazol-1-yl)pyrimidin-4-yl)-3-methylmorpholine (D71)

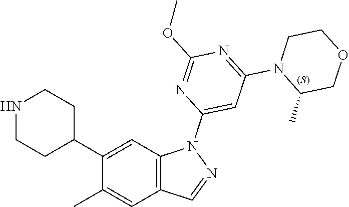

The title compounds were prepared by a procedure similar to those described for D17 starting from a solution of (S)-tert-butyl 4-(1-(2-methoxy-6-(3-methylmorpholino)pyrimidin-4-yl)-5-methyl-1H-indazol-6-yl)piperidine-1-carboxylate in EtOAc and HCl/EtOAc.

LC-MS[mobile phase: from 90% water (0.1% FA) and 10% CH₃CN (0.1% FA) to 5% water (0.1% FA) and 95% CH₃CN (0.1% FA) in 2.0 min]: Rt=1.21 min; MS Calcd.: 422.2, MS Found: 423.5 [M+H]⁺.

### Description 72

### (S)-ethyl 2-(4-(1-(2-methoxy-6-(3-methylmorpholino)pyrimidin-4-yl)-5-methyl-1H-indazol-6-yl)piperidin-1-yl)acetate (D72)

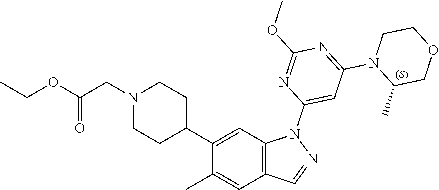

The title compound was prepared by a procedure similar to those described for D18 starting from ethyl 2-bromoacetate and a solution of (S)-4-(2-methoxy-6-(5-methyl-6-(piperidin-4-yl)-1H-indazol-1-yl)pyrimidin-4-yl)-3-methylmorpholine and Et₃N in DMF.

LC-MS [mobile phase: from 90% water (0.1% FA) and 10% CH₃CN (0.1% FA) to 5% water (0.1% FA) and 95% CH₃CN (0.1% FA) in 2.0 min]: Rt=1.34 min; MS Calcd.: 508.3, MS Found: 509.5 [M+H]⁺.

### Description 73

### 4-(azetidin-1-yl)-6-iodo-2-methoxypyrimidine (D73)

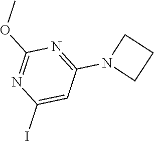

A mixture of 4,6-diiodo-2-methoxypyrimidine (362 mg, 1.00 mol), azetidine (86.0 mg, 1.50 mol) and DIEA (388 g, 3.00 mmol) in THF (10.0 mL) and ⁱ-PrOH (10.0 mL) was stirred at rt overnight. The reaction solution was concentrated and purified by chromatography (PE:EtOAc=2:1) to give the title product (269 mg, yield: 92.0%) as a white solid.

LC-MS [mobile phase: from 80% water (0.1% TFA) and 20% CH₃CN (0.1% TFA) to 5% water (0.1% TFA) and 95% CH₃CN (0.1% TFA) in 2.0 min]: Rt=1.38 min; MS Calcd.: 291.0, MS Found: 292.1 [M+H]⁺.

### Description 74

### tert-butyl 4-(1-(6-(azetidin-1-yl)-2-methoxypyrimidin-4-yl)-5-methyl-1H-indazol-6-yl)-3-fluoropiperidine-1-carboxylate (D74)

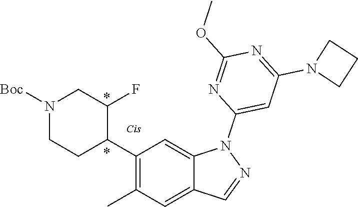 [SMILES: COc1nc(N2CCC2)cc(-n2ncc3cc(C)c(C4CCN(C(=O)OC(C)(C)C)CC4F)cc32)n1]

To a suspension of cis-tert-butyl 3-fluoro-4-(5-methyl-1H-indazol-6-yl)piperidine-1-carboxylate (D9, Peak 1, 60 mg, 0.18 mmol), 4-(azetidin-1-yl)-6-iodo-2-methoxypyri-midine (63 mg, 0.22 mmol), CuI (34 mg, 0.18 mmol), K₃PO₄ (76 mg, 0.36 mmol) in toluene/THF (5.0 mL/1 mL) was added DMEDA (32 mg, 0.36 mmol). The resulting mixture was degassed with N₂ three times, then stirred at 80° C. for 2 hour. The reaction mixture was diluted with EtOAc (20 mL), washed with sat. NH₄Cl (20 mL) and brine (20 mL). The organic solution was dried over anhydrous Na₂SO₄ and concentrated. The residue was purified by prep-TLC (PE:EtOAc=2:1) to give the title product (58 mg, yield: 65%) as a pale yellow solid.

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=1.30 min; MS Calcd.: 496.3, MS Found: 497.3 [M+H]⁺.

### Description 75

### 1-(6-(azetidin-1-yl)-2-methoxypyrimidin-4-yl)-6-(3-fluoropiperidin-4-yl)-5-methyl-1H-indazole (D75)

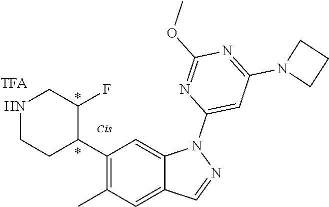 [SMILES: COc1nc(N2CCC2)cc(-n2ncc3cc(C)c(C4CCNCC4F)cc32)n1]

To a solution of tert-butyl 4-(1-(6-(azetidin-1-yl)-2-methoxypyrimidin-4-yl)-5-methyl-1H-indazol-6-yl)-3-fluoropiperidine-1-carboxylate (D74, 58 mg, 0.12 mmol) in CH₂Cl₂ (5.0 mL) was added dropwise TFA (1.0 mL). The reaction solution was stirred at rt for 60 min and concentrated to give the title product (59 mg, yield: 100%) as a pale yellow solid.

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=1.23 min; MS Calcd.: 396.2, MS Found: 397.4 [M+H]⁺.

### Description 76

### ethyl 2-(4-(1-(6-(azetidin-1-yl)-2-methoxypyrimidin-4-yl)-5-methyl-1H-indazol-6-yl)-3-fluoropiperidin-1-yl)acetate (D76, from Peak 1)

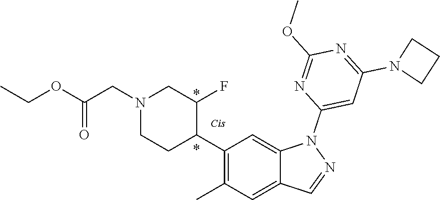 [SMILES: CCOC(=O)CN1CCC(c2cc3c(cnn3-c3cc(N4CCC4)nc(OC)n3)cc2C)C(F)C1]

To a solution of 1-(6-(azetidin-1-yl)-2-methoxypyrimidin-4-yl)-6-(3-fluoropiperidin-4-yl)-5-methyl-1H-indazole (D75, 59 mg, 0.12 mmol) and Et₃N (60 mg, 0.60 mmol) in DMF (2.0 mL) was slowly added ethyl 2-bromoacetate (40 mg, 0.24 mmol). The reaction solution was stirred at rt for 60 min, then quenched with sat. NH₄Cl, diluted with EtOAc (20 mL), washed with brine (50 mL), dried and concentrated. The residue was purified by prep-TLC (PE:EtOAc=1:1) to give the title product (49 mg, yield: 88%) as a pale yellow solid.

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=1.66 min; MS Calcd.: 482.2, MS Found: 483.4 [M+H]⁺.

### Description 77

### tert-butyl 4-(1-(6-(azetidin-1-yl)-2-methoxypyrimidin-4-yl)-5-methyl-1H-indazol-6-yl)-3-fluoropiperidine-1-carboxylate (D77)

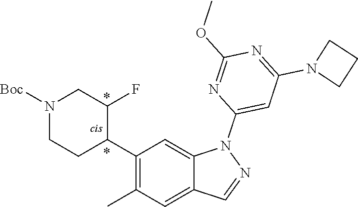 [SMILES: COc1nc(N2CCC2)cc(-n2ncc3cc(C)c(C4CCN(C(=O)OC(C)(C)C)CC4F)cc32)n1]

The title compound was prepared by a procedure similar to that described for D74 starting from a suspension of cis-tert-butyl 3-fluoro-4-(5-methyl-1H-indazol-6-yl)piperidine-1-carboxylate (D10, Peak 2), 4-(azetidin-1-yl)-6-iodo-2-methoxypyrimidine (D₇₃), CuI and K₃PO₄ in toluene/THF and DMEDA.

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=1.30 min; MS Calcd.: 496.3, MS Found: 497.3 [M+H]⁺.

### Description 78

### 1-(6-(azetidin-1-yl)-2-methoxypyrimidin-4-yl)-6-(3-fluoropiperidin-4-yl)-5-methyl-1H-indazole (D78, from Peak 2)

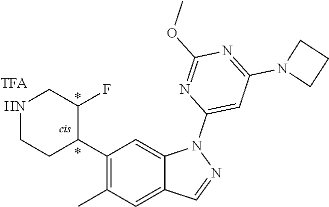 [SMILES: COc1nc(N2CCC2)cc(-n2ncc3cc(C)c(C4CCNCC4F)cc32)n1]

The title compound was prepared by a procedure similar to that described for D74 starting from a solution of tert-butyl 4-(1-(6-(azetidin-1-yl)-2-methoxypyrimidin-4-yl)-5-methyl-1H-indazol-6-yl)-3-fluoropiperidine-1-carboxylate (D77) in CH₂Cl₂ and TFA.

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=1.23 min; MS Calcd.: 396.2, MS Found: 397.4 [M+H]⁺.

### Description 79

### ethyl 2-(4-(1-(6-(azetidin-1-yl)-2-methoxypyrimidin-4-yl)-5-methyl-1H-indazol-6-yl)-3-fluoropiperidin-1-yl)acetate (D79, from Peak 2)

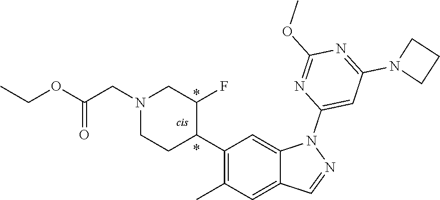 [SMILES: CCOC(=O)CN1CCC(c2cc3c(cnn3-c3cc(N4CCC4)nc(OC)n3)cc2C)C(F)C1]

The title compound was prepared by a procedure similar to that described for D75 starting from ethyl 2-bromoacetate (37 mg, 0.22 mmol) was slowly added to the solution of 1-(6-(azetidin-1-yl)-2-methoxypyrimidin-4-yl)-6-(3-fluoropiperidin-4-yl)-5-methyl-1H-indazole (D78) and Et₃N in DMF.

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=1.66 min; MS Calcd.: 482.2, MS Found: 483.4 [M+H]⁺.

### Description 80

### tert-butyl 4-(5-chloro-1H-indazol-6-yl) piperidine-1-carboxylate (D80)

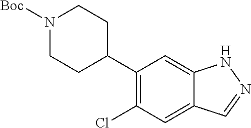 [SMILES: CC(C)(C)OC(=O)N1CCC(c2cc3[nH]ncc3cc2Cl)CC1]

To a solution of tert-butyl 6-(1-(tert-butoxycarbonyl)piperidin-4-yl)-5-chloro-1H-indazole-1-carboxylate (D117, 600 mg, 1.38 mmol) in MeOH (40.0 mL) was added aq. NaOH (1 M, 40.0 mL) at rt. The reaction mixture was stirred at rt overnight, diluted with CH₂Cl₂ (50 mL×3). The combined organic layers were washed with brine (50 mL), dried over anhydrous Na₂SO₄, filtered and concentrated to dryness. The residue was purified by silica gel chromatography eluted with PE:EtOAc=1:1 to afford the title product as a white solid (400 mg, yield: 86.0%).

LC-MS [mobile phase: from 50% water (0.1% TFA) and 50% CH₃CN (0.1% T FA) to 5% water (0.1% TFA) and 95% CH₃CN (0.1% TFA) in 2.6 min]: Rt=1.57 min; MS Calcd: 335.8, MS Found: 336.1 [M+H]⁺.

### Description 81

### tert-butyl 4-(5-chloro-1-(6-chloro-2-methylpyrimidin-4-yl)-1H-indazol-6-yl) piperidi-ne-1-carboxylate (D81)

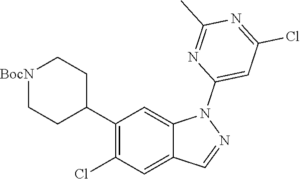 [SMILES: Cc1nc(Cl)cc(-n2ncc3cc(Cl)c(C4CCN(C(=O)OC(C)(C)C)CC4)cc32)n1]

A mixture of tert-butyl 4-(5-chloro-1H-indazol-6-yl) piperidine-1-carboxylate (2.00 g, 6.00 mmol), 4,6-dichloro-2-methylpyrimidine (978 mg, 6.00 mmol) and Cs₂CO₃ (5.90 g, 18.0 mmol) in DMF (80.0 mL) was stirred at 50° C. for 5 h. The reaction mixture was diluted with water (50 mL) and extracted with EtOAc (100 mL×3). The combined organic layers were washed with water (50 mL×3) and brine (50 mL), dried, filtered and concentrated. The residue was purified by silica gel chromatography eluted with (PE:EtOAc=5:1) to give the crude product. The crude was recrystallized from MeOH to give the title product as a white solid (380 mg, 13.0% yield).

LC-MS [mobile phase: from 20% water (0.1% TFA) and 80% CH₃CN (0.1% TFA) to 5% water (0.1% T FA) and 95% CH₃CN (0.1% TFA) in 10 min]: Rt=3.44 min; MS Calcd: 461.1, MS Found: 462.1 [M+H]⁺.

### Description 82

### 5-chloro-1-(6-chloro-2-methylpyrimidin-4-yl)-6-(piperidin-4-yl)-1H-indazole (D82)

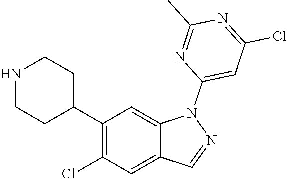 [SMILES: Cc1nc(Cl)cc(-n2ncc3cc(Cl)c(C4CCNCC4)cc32)n1]

To a solution of tert-butyl 4-(5-chloro-1-(6-chloro-2-methylpyrimidin-4-yl)-1H-indazol-6-yl) piperidine-1-carboxylate (380 mg, 0.820 mmol) in CH₂Cl₂ (80.0 mL) was added 2,2,2-trifluoroacetic acid (10 mL). The reaction mixture was stirred at rt overnight, diluted with NH₃.H₂O (15 mL) and extracted with CH₂Cl₂ (100 mL×2). The combined organic layers were washed with brine, dried over anhydrous Na₂SO₄ and filtered. The filtrate was concentrated to dryness to give the title product as a white solid (310 mg, yield: 100%).

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=0.81 min; MS Calcd: 361.0, MS Found: 362.0 [M+H]⁺.

### Description 83

### 1-(4-(5-chloro-1-(6-chloro-2-methylpyrimidin-4-yl)-1H-indazol-6-yl) piperidin-1-yl)propan-2-one (D83)

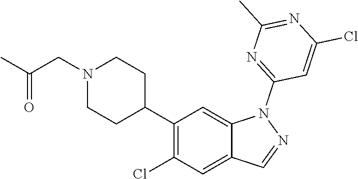 [SMILES: CC(=O)CN1CCC(c2cc3c(cnn3-c3cc(Cl)nc(C)n3)cc2Cl)CC1]

To a solution of 5-chloro-1-(6-chloro-2-methylpyrimidin-4-yl)-6-(piperidin-4-yl)-1H-indazole (300 mg, 0.830 mmol) and 1-bromopropan-2-one (164 mg, 1.20 mmol) in DMF (40.0 mL) was added Et₃N (242 mg, 2.40 mmol). The reaction mixture was stirred at rt overnight, concentrated, diluted with H₂O (50.0 mL) and extracted with EtOAc (50 mL×3). The combined organic layers were washed with water (50 mL×3) and brine, dried over anhydrous Na₂SO₄, filtered and concentrated. The residue was purified by silica gel chromatography eluted with EtOAc to give the title product as a white solid (240 mg, yield: 69.0%).

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=0.82 min; MS Calcd: 417.1, MS Found: 418.1 [M+H]⁺.

### Description 84

### (R)-1-(4-(5-chloro-1-(6-(2-(hydroxymethyl)morpholino)-2-methylpyrimidin-4-yl)-1H-indazol-6-yl)piperidin-1-yl)propan-2-one (D84)

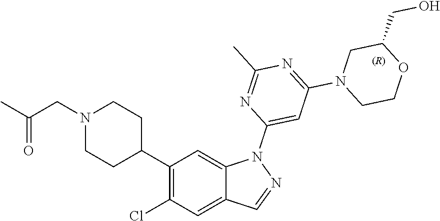

To a solution of 1-(4-(5-chloro-1-(6-chloro-2-methylpyrimidin-4-yl)-1H-indazol-6-yl) piperidin-1-yl) propan-2-one (120 mg, 0.290 mmol) and (R)-morpholin-2-ylmethanol hydrochloride (D114, 44.0 mg, 0.290 mmol) in DMF (30.0 mL) was added DIEA (187 mg, 1.45 mmol) at rt. The reaction mixture was stirred at 80° C. overnight, cooled to room temperature, diluted with water (50 mL) and extracted with EtOAc (50 mL×3). The combined organic layers were washed with water (50 mL×3) and brine (50 mL), dried, filtered and concentrated to give the title product as a yellow solid (110 mg, yield: 76.0%).

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=0.78 min; MS Calcd.: 498.2, MS Found: 499.2 [M+H]⁺.

### Description 85

### 1-(4-(5-chloro-1-(6-((R)-2-(hydroxymethyl)morpholino)-2-methylpyrimidin-4-yl)-1H-indazol-6-yl)piperidin-1-yl)propan-2-ol (D85)

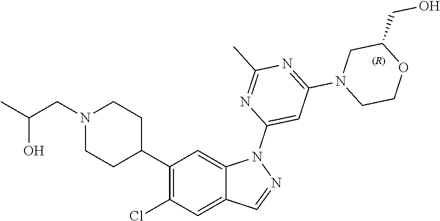

To a solution of (R)-1-(4-(5-chloro-1-(6-(2-(hydroxymethyl)morpholino)-2-methylpyrim-idin-4-yl)-1H-indazol-6-yl)piperidin-1-yl)propan-2-one (110 mg, 0.220 mmol) in MeOH (30.0 mL) was added NaBH₄ (24.0 mg, 0.660 mmol) at 0° C. The reaction mixture was stirred at 0° C. for 3 h, diluted with H₂O (50.0 mL) and extracted with EtOAc (50 mL×3). The combined organic layers were washed with water and brine, dried over anhydrous Na₂SO₄, filtered and concentrated. The residue was purified by silica gel chromatography eluted with (EtOAc:MeOH=20:1) to give the title product as a white solid (78.0 mg, yield: 70.0%).

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=0.77 min; MS Calcd: 500.2, MS Found: 501.2 [M+H]⁺.

### Description 86

### (S)-1-(4-(5-chloro-1-(6-(2-(hydroxymethyl)morpholino)-2-methylpyrimidin-4-yl)-1H-indazol-6-yl)piperidin-1-yl)propan-2-one (D86)

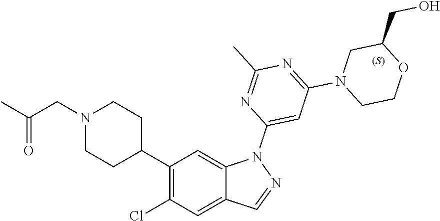

To a solution of 1-(4-(5-chloro-1-(6-chloro-2-methylpyrimidin-4-yl)-1H-indazol-6-yl) piperidin-1-yl) propan-2-one (120 mg, 0.290 mmol) and (S)-morpholin-2-ylmethanol hydrochloride (44.0 mg, 0.290 mmol) in DMF (30.0 mL) was added DIEA (187 mg, 1.45 mmol) at rt. The reaction mixture was stirred at 80° C. overnight, cooled to room temperature, diluted with water (50 mL) and extracted with EtOAc (50 mL×3). The combined organic layers were washed with water (50 mL×3) and brine (50 mL), dried and filtered. The filtrate was concentrated to give the title product as a yellow solid (110 mg, yield: 76.0%).

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=0.78 min; MS Calcd.: 498.2, MS Found: 499.2 [M+H]⁺.

### Description 87

### cis-tert-butyl 4-(1-(6-chloro-2-methylpyrimidin-4-yl)-5-methyl-1H-indazol-6-yl)-3-fluoropiperidine-1-carboxylate (D87)

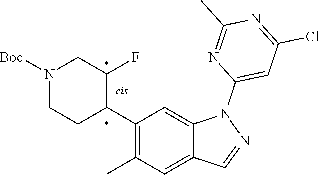 [SMILES: Cc1nc(Cl)cc(-n2ncc3cc(C)c(C4CCN(C(=O)OC(C)(C)C)CC4F)cc32)n1]

A mixture of cis-tert-butyl 3-fluoro-4-(5-methyl-1H-indazol-6-yl)piperidine-1-carboxylate (D9, peak 1) (500 mg, 1.50 mmol) and 4,6-dichloro-2-methylpyrimidine (300 mg, 1.84 mmol) and Cs₂CO₃ (1.60 g, 4.91 mmol) in DMF (20 mL) was stirred at 40° C. for 2 h, diluted with EtOAc (100 mL) and washed with brine (100 mL×3). The organic layer was dried and concentrated. The residue was purified by column (PE:EtOAc=10:1) to give the title product as an off-white solid (500 mg, 72.0% yield). The solid was recrystallized from MeOH to give the pure product as a white solid (342 mg, 50% yield)

LC-MS [mobile phase: from 50% water (0.1% TFA) and 50% CH₃CN (0.1% TFA) to 5% water (0.1% TFA) and 95% CH₃CN (0.1% TFA) in 2.0 min]: Rt=1.81 min; MS Calcd.: 459.2, MS Found: 460.3 [M+H]⁺.

### Description 88

### 1-(6-chloro-2-methylpyrimidin-4-yl)-6-(cis-3-fluoropiperidin-4-yl)-5-methyl-1H-indazole hydrochloride (D88)

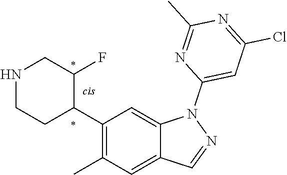 [SMILES: Cc1nc(Cl)cc(-n2ncc3cc(C)c(C4CCNCC4F)cc32)n1]

To a mixture of cis-tert-butyl 4-(1-(6-chloro-2-methylpyrimidin-4-yl)-5-methyl-1H-indazol-6-yl)-3-fluoropiperidine-1-carboxylate (D87, 300 mg, 0.650 mmol) in MeOH (10 mL) was added HCl/EtOAc (5 mL, 6 N). The reaction mixture was stirred at rt for 1 h and concentrated to give the title product as a white solid (280 mg)

LC-MS [mobile phase: from 50% water (0.1% TFA) and 50% CH₃CN (0.1% TFA) to 5% water (0.1% TFA) and 95% CH₃CN (0.1% TFA) in 2.0 min]: Rt=0.37 min; MS Calcd.: 359.1, MS Found: 360.3 [M+H]⁺.

### Description 89

### 1-(cis-4-(1-(6-chloro-2-methylpyrimidin-4-yl)-5-methyl-1H-indazol-6-yl)-3-fluoropi-peridin-1-yl)propan-2-one (D89)

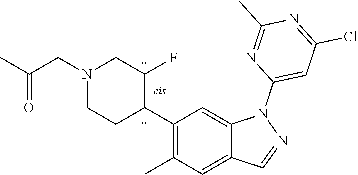 [SMILES: CC(=O)CN1CCC(c2cc3c(cnn3-c3cc(Cl)nc(C)n3)cc2C)C(F)C1]

To a solution of 1-(6-chloro-2-methylpyrimidin-4-yl)-6-(cis-3-fluoropiperidin-4-yl)-5-methyl-1H-indazole hydrochloride (D88, 260 mg, 0.600 mmol) in DMF (10 mL) were added 1-bromopropan-2-one (274 mg, 2.00 mmol) and Et₃N (1 mL). The reaction mixture was stirred at rt for 1 h, diluted with EtOAc (50 mL), washed with brine (60 mL×3), dried and concentrated to give the title product as an off-white solid (310 mg)

LC-MS [mobile phase: from 80% water (0.1% TFA) and 20% CH₃CN (0.1% TFA) to 5% water (0.1% T FA) and 95% CH₃CN (0.1% TFA) in 2.0 min]: Rt=1.37 min; MS Calcd.: 415.2, MS Found: 416.2 [M+H]⁺.

### Description 90

### 1-(cis-3-fluoro-4-(1-(6-((R)-2-(hydroxymethyl)morpholino)-2-methylpyrimidin-4-yl)-5-methyl-1H-indazol-6-yl)piperidin-1-yl)propan-2-one (D90)

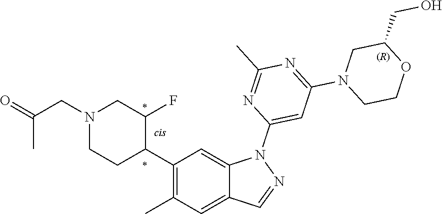

A solution of 1-(cis-4-(1-(6-chloro-2-methylpyrimidin-4-yl)-5-methyl-1H-indazol-6-yl)-3-fluoropiperidin-1-yl)propan-2-one (D89, 70.7 mg, 0.170 mmol), (R)-morpholin-2-ylmeth-anol hydrochloride (D116, 153 mg, 1.00 mmol) and Et₃N (1 mL) in NMP (10 mL) was stirred at 60° C. for 2 h, diluted with EtOAc (50 mL), washed with brine (50 mL×3). The organic solution was dried and concentrated. The residue was purified by prep-TLC (MeOH/DCM=1/10) to give the title product as a white solid (79.0 mg, 99.0% yield)

LC-MS [mobile phase: from 80% water (0.1% TFA) and 20% CH₃CN (0.1% TFA) to 5% water (0.1% TFA) and 95% CH₃CN (0.1% TFA) in 2.0 min]: Rt=1.09 min; MS Calcd.: 496.3, MS Found: 497.3 [M+H]⁺.

### Description 91

### 1-(cis-3-fluoro-4-(1-(6-((S)-2-(hydroxymethyl)morpholino)-2-methylpyrimidin-4-yl)-5-methyl-1H-indazol-6-yl)piperidin-1-yl)propan-2-one (D91)

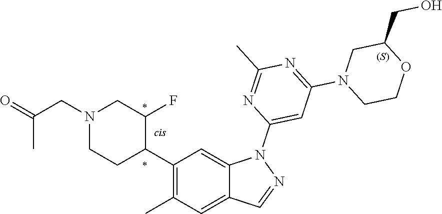

The title compound was prepared by a procedure similar to that described for D90 starting from a solution of 1-(cis-4-(1-(6-chloro-2-methylpyrimidin-4-yl)-5-methyl-1H-indazol-6-yl)-3-fluoropiperidin-1-yl)propan-2-one (D89), (S)-morpholin-2-ylmethanol hydrochloride and Et₃N in NMP at 60° C. for 2 h.

LC-MS [mobile phase: from 80% water (0.1% TFA) and 20% CH₃CN (0.1% TFA) to 5% water (0.1% TFA) and 95% CH₃CN (0.1% TFA) in 2.0 min]: Rt=1.05 min; MS Calcd.: 496.3, MS Found: 497.3 [M+H]⁺.

### Description 92

### cis-tert-butyl 4-(1-(6-chloro-2-methylpyrimidin-4-yl)-5-methyl-1H-indazol-6-yl)-3-fluo-ropiperidine-1-carboxylate (D92)

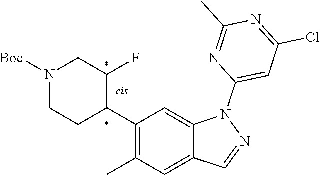 [SMILES: Cc1nc(Cl)cc(-n2ncc3cc(C)c(C4CCN(C(=O)OC(C)(C)C)CC4F)cc32)n1]

The title compound was prepared by a procedure similar to that described for D87 starting from a mixture of cis-tert-butyl 3-fluoro-4-(5-methyl-1H-indazol-6-yl)piperidine-1-carboxylate (D10, peak 2), 4,6-dichloro-2-methylpyrimidine and Cs₂CO₃ in DMF at 40° C. for 2 h.

LC-MS [mobile phase: from 50% water (0.1% TFA) and 50% CH₃CN (0.1% TFA) to 5% water (0.1% TFA) and 95% CH₃CN (0.1% TFA) in 2.0 min]: Rt=1.81 min; MS Calcd: 459.2, MS Found: 460.3 [M+H]⁺.

### Description 93

### 1-(6-chloro-2-methylpyrimidin-4-yl)-6-(cis-3-fluoropiperidin-4-yl)-5-methyl-1H-indazole hydrochloride (D93)

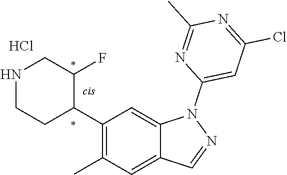 [SMILES: Cl ; Cc1nc(Cl)cc(-n2ncc3cc(C)c(C4CCNCC4F)cc32)n1]

The title compound was prepared by a procedure similar to that described for D88 starting from a mixture of cis-tert-butyl 4-(1-(6-chloro-2-methylpyrimidin-4-yl)-5-methyl-1H-indazol-6-yl)-3-fluoropiperidine-1-carboxylate in MeOH and HCl/EtOAc at rt for 1 h.

LC-MS [mobile phase: from 50% water (0.1% TFA) and 50% CH₃CN (0.1% TFA) to 5% water (0.1% TFA) and 95% CH₃CN (0.1% TFA) in 2.0 min]: Rt=0.37 min; MS Calcd.: 359.1, MS Found: 360.3 [M+H]⁺.

### Description 94

### 1-(cis-4-(1-(6-chloro-2-methylpyrimidin-4-yl)-5-methyl-1H-indazol-6-yl)-3-fluorop-iperidin-1-yl)propan-2-one (D94)

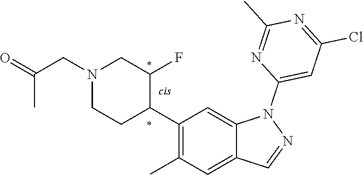 [SMILES: CC(=O)CN1CCC(c2cc3c(cnn3-c3cc(Cl)nc(C)n3)cc2C)C(F)C1]

The title compound was prepared by a procedure similar to that described for D89 starting from a solution of 1-(6-chloro-2-methylpyrimidin-4-yl)-6-(cis-3-fluoropiperidin-4-yl)-5-methyl-1H-indazole hydrochloride in DMF, 1-bromopropan-2-one and Et₃N at rt for 1 h.

LC-MS [mobile phase: from 80% water (0.1% TFA) and 20% CH₃CN (0.1% TFA) to 5% water (0.1% TFA) and 95% CH₃CN (0.1% TFA) in 2.0 min]: Rt=1.37 min; MS Calcd.: 415.2, MS Found: 416.2 [M+H]⁺.

### Description 95

### 1-(cis-3-fluoro-4-(1-(6-((R)-2-(hydroxymethyl)morpholino)-2-methylpyrimidin-4-yl)-5-methyl-1H-indazol-6-yl)piperidin-1-yl)propan-2-one (D95)

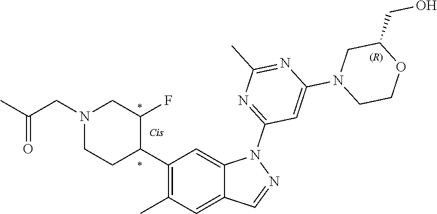

The title compound was prepared by a procedure similar to that described for D90 starting from a solution of 1-(4-(1-(6-chloro-2-methylpyrimidin-4-yl)-5-methyl-1H-indazol-6-yl)-cis-3-fluoropiperidin-1-yl)propan-2-one (D94), (R)-morpholin-2-ylmethanol hydrochloride and Et₃N in NMP at 60° C. for 2 h.

LC-MS [mobile phase: from 80% water (0.1% TFA) and 20% CH₃CN (0.1% TFA) to 5% water (0.1% TFA) and 95% CH₃CN (0.1% TFA) in 2.0 min]: Rt=1.14 min; MS Calcd.: 496.26, MS Found: 497.3 [M+H]⁺.

### Description 96

### 1-(cis-3-fluoro-4-(1-(6-((S)-2-(hydroxymethyl)morpholino)-2-methylpyrimidin-4-yl)-5-methyl-1H-indazol-6-yl)piperidin-1-yl)propan-2-one (D96)

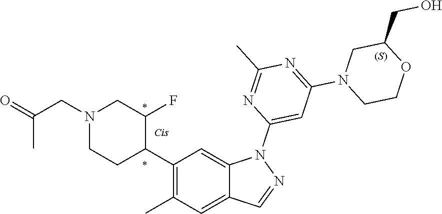

A solution of 1-(cis-4-(1-(6-chloro-2-methylpyrimidin-4-yl)-5-methyl-1H-indazol-6-yl) 3-fluoropiperidin-1-yl)propan-2-one (D94, 75.0 mg, 0.180 mmol), (S)-morpholin-2-ylme-thanol hydrochloride (138 mg, 0.900 mmol) and Et₃N (125 mg) in NMP (5.00 mL) was stirred at 60° C. for 2 h. The reaction solution was diluted with sat. NH₄Cl (80.0 mL) and extracted with EtOAc (60 mL×3). The combined organic layers were dried and concentrated. The residue was purified by silico gel chromatograohy (MeOH/CH₂Cl₂=1/15) to give the title product as a white solid (120 mg)

LC-MS [mobile phase: from 80% water (0.1% TFA) and 20% CH₃CN (0.1% TFA) to 5% water (0.1% TFA) and 95% CH₃CN (0.1% TFA) in 2.0 min]: Rt=1.14 min; MS Calcd.: 496.3, MS Found: 497.3 [M+H]⁺.

### Description 97

### tert-butyl 4-(1-(6-(3-hydroxyazetidin-1-yl)-2-methylpyrimidin-4-yl)-5-methyl-1H-in-daz-ol-6-yl)piperidine-1-carboxylate (D97)

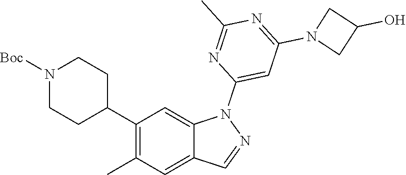 [SMILES: Cc1nc(N2CC(O)C2)cc(-n2ncc3cc(C)c(C4CCN(C(=O)OC(C)(C)C)CC4)cc32)n1]

A suspension of 1-(6-iodo-2-methylpyrimidin-4-yl)azetidin-3-ol (291 mg, 1.00 mmol), tart-butyl 4-(5-methyl-1H-indazol-6-yl)piperidine-1-carboxylate (315 mg, 1.00 mmol), CuI (38.0 mg, 0.200 mmol), K₃PO₄ (424 mg, 2.00 mmol) and N,N′-dimethylcyclohexane-1,2-diamine (56.0 mg, 0.400 mmol) in toluene (3.00 mL) was stirred at 100° C. overnight, diluted with EtOAc (30 mL), washed with NH₃H₂O (15 mL×3). The organic layer was dried over Na₂SO₄, filtered and concentrated. The crude was purified by flash chromatography (petroleum ether/EtOAc=1:1) to give the title compound (420 mg, 88%) as a white solid.

¹HNMR (400 MHz, CDCl₃): δ 8.75 (s, 1H), 8.06 (s, 1H), 7.50 (s, 1H), 6.59 (s, 1H), 4.84-4.81 (m, 1H), 4.43-4.29 (m, 4H), 4.02-3.98 (m, 2H), 2.98-2.85 (m, 3H), 2.61 (s, 3H), 2.47 (s, 3H), 1.89-1.86 (m, 2H), 1.78-1.72 (m, 3H), 1.50 (s, 9H).

### Description 98

### 1-(2-methyl-6-(5-methyl-6-(piperidin-4-yl)-1H-indazol-1-yl)pyrimidin-4-yl)azetidin-3-ol (D98)

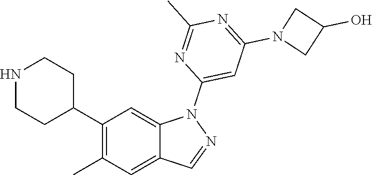 [SMILES: Cc1nc(N2CC(O)C2)cc(-n2ncc3cc(C)c(C4CCNCC4)cc32)n1]

To a solution of tert-butyl 4-(1-(6-(3-hydroxyazetidin-1-yl)-2-methylpyrimidin-4-yl)-5-methyl-1H-indazol-6-yl)piperidine-1-carboxylate (420 mg, 0.880 mmol) in MeOH (2.00 mL) was added HCl/MeOH (2 M, 1 mL). The reaction mixture was stirred at room temperature for 3 hours and concentrated to give the title product (332 mg, 99.0%) as a white solid.

LCMS [column: Phenomenex Kinetex 5 μm EVO, C₁₈; column size: 4.6×50 mm; mobile phase: B (CH₃CN), A (0.02% NH₄Ac in water); gradient (B %) in 4 mins]: Rt=1.580 min, MS Calcd.: 378, MS Found: 379 [M+H]⁺.

### Description 99

### 1-fluoro-3-(4-(5-methyl-1-(tetrahydro-2H-pyran-2-yl)-1H-indazol-6-yl)piperidin-1-yl)propan-2-ol (D99)

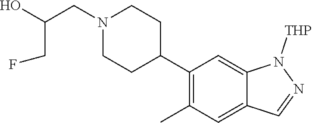

A mixture of 5-methyl-6-(piperidin-4-yl)-1-(tetrahydro-2H-pyran-2-yl)-1H-indazole (D114, 787 mg, 2.63 mmol), 2-(fluoromethyl)oxirane (1.00 g, 13.2 mmol) and Cs₂CO₃ (2.60 g, 7.89 mmol) in DMF (10.0 mL) was stirred at 80° C. in a sealed tube overnight. The resulting mixture was diluted with EtOAc (30.0 mL), washed with water (50 mL×2). The organic solution was dried over anhydrous Na₂SO₄ and concentrated. The residue was purified by silica gel column (CH₂Cl₂:MeOH=30:1-10:1) to give the title product (420 mg, yield: 43%) as a yellow oil.

LC-MS [mobile phase: from 70% water (0.1% TFA) and 30% CH₃CN (0.1% TFA) to 5% water (0.1% TFA) and 95% CH₃CN (0.1% TFA) in 9.0 min]: Rt=3.04 min; MS Calcd: 375.2, MS Found: 376.4 [M+H]⁺.

### Description 100

### 1-fluoro-3-(4-(5-methyl-1H-indazol-6-yl)piperidin-1-yl)propan-2-ol (D100)

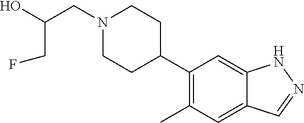 [SMILES: Cc1cc2cn[nH]c2cc1C1CCN(CC(O)CF)CC1]

To a solution of 1-fluoro-3-(4-(5-methyl-1-(tetrahydro-2H-pyran-2-yl)-1H-indazol-6-yl)p-iperidin-1-yl)propan-2-ol (D99, 420 mg, 1.12 mmol) in CH₂Cl₂ (10.0 mL) was added dropwise TFA (2.00 mL). The reaction solution was stirred at room temperature overnight, concentrated and diluted with water (20.0 mL) and MeOH (5.00 mL). The resulting mixture was basified to pH-9 with solid K₂CO₃, then extracted with CH₂Cl₂ (20 mL×3). The combined organic layers were dried over anhydrous Na₂SO₄ and concentrated. The residue was purified by silica gel column (CH₂Cl₂:MeOH=5:1) to give the title product (185 mg, yield: 57.0%) as a yellow solid.

LC-MS [mobile phase: from 70% water (0.1% TFA) and 30% CH₃CN (0.1% TFA) to 5% water (0.1% TFA) and 95% CH₃CN (0.1% TFA) in 9.0 min]: Rt=3.85 min; MS Calcd: 291.2, MS Found: 292.3 [M+H]⁺.

### Description 101

### methyl 2-hydroxy-3-(4-(5-methyl-1-(tetrahydro-2H-pyran-2-yl)-1H-indazol-6-yl)p-eridin-1-yl)propanoate (D101)

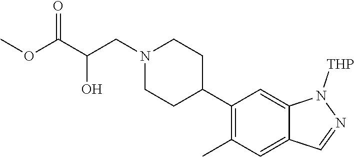

A mixture of 5-methyl-6-(piperidin-4-yl)-1-(tetrahydro-2H-pyran-2-yl)-1H-indazole (D114, 4.40 g, 14.7 mmol), methyl oxirane-2-carboxylate (4.50 g, 44.1 mmol) and Cs₂CO₃ (14.4 g, 44.1 mmol) in DMF (40 mL) was stirred at 80° C. in a sealed tube overnight. The resulting mixture was diluted with EtOAc (80.0 mL) and washed with water (100 mL×2). The organic solution was dried over anhydrous Na₂SO₄ and concentrated. The residue was purified by silica gel column (CH₂Cl₂:MeOH=30:1) to give the title product (1.70 g, yield: 29.0%) as a yellow solid.

LC-MS [mobile phase: from 70% water (0.1% TFA) and 30% CH₃CN (0.1% TFA) to 5% water (0.1% TFA) and 95% CH₃CN (0.1% TFA) in 9.0 min]: Rt=3.28 min; MS Calcd: 401.2, MS Found: 402.4 [M+H]⁺.

### Description 102

### methyl 2-fluoro-3-(4-(5-methyl-1-(tetrahydro-2H-pyran-2-yl)-1H-indazol-6-yl)piper-idin-1-yl)propanoate (D102)

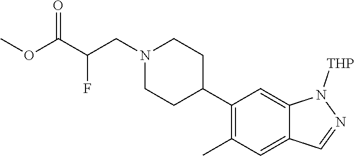

To a solution of methyl 2-hydroxy-3-(4-(5-methyl-1-(tetrahydro-2H-pyran-2-yl)-1H-indazol-6-yl)piperidin-1-yl)propanoate (D101, 1.70 g, 4.23 mmol) in CH₂Cl₂ (15.0 mL) was added dropwise a solution of DAST (2.00 g, 12.7 mmol) in CH₂Cl₂ (5 mL) at −60° C. under N₂. The reaction mixture was stirred at room temperature for 4 h, quenched with sat. NaHCO₃ and washed with brine (20 mL). The separated organic part was dried over anhydrous Na₂SO₄ and concentrated. The residue was purified by silica gel column (CH₂Cl₂:MeOH=100:1) to give the title product (620 mg, yield: 36.0%) as a yellow oil.

LC-MS [mobile phase: from 70% water (0.1% TFA) and 30% CH₃CN (0.1% TFA) to 5% water (0.1% TFA) and 95% CH₃CN (0.1% TFA) in 2.0 min]: Rt=1.10 min; MS Calcd: 403.2, MS Found: 404.4 [M+H]⁺.

### Description 103

### 2-fluoro-3-(4-(5-methyl-1-(tetrahydro-2H-pyran-2-yl)-1H-indazol-6-yl)piperidin-1-yl)propan-1-ol (D103)

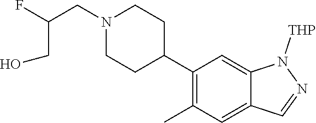

To a solution of methyl 2-fluoro-3-(4-(5-methyl-1-(tetrahydro-2H-pyran-2-yl)-1H-indazol-6-yl)piperidin-1-yl)propanoate (D102, 620 mg, 1.54 mmol) in MeOH (10.0 mL) was added NaBH₄ (174 mg, 4.61 mmol). The reaction mixture was stirred at room temperature for 30 min, quenched with sat. NaHCO₃, diluted with DCM (30 mL) and washed with brine (20 mL×2). The separated organic part was dried over anhydrous Na₂SO₄ and concentrated to give the title product (560 mg, yield: 97.0%) as a yellow solid.

LC-MS [mobile phase: from 70% water (0.1% TFA) and 30% CH₃CN (0.1% TFA) to 5% water (0.1% TFA) and 95% CH₃CN (0.1% TFA) in 2.0 min]: Rt=1.02 min; MS Calcd: 375.2, MS Found: 376.4 [M+H]⁺.

### Description 104

### 2-fluoro-3-(4-(5-methyl-1H-indazol-6-yl)piperidin-1-yl)propan-1-ol (D104)

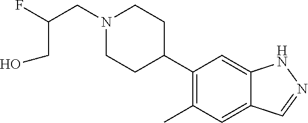 [SMILES: Cc1cc2cn[nH]c2cc1C1CCN(CC(F)CO)CC1]

To a solution of 2-fluoro-3-(4-(5-methyl-1-(tetrahydro-2H-pyran-2-yl)-1H-indazol-6-yl)piperidin-1-yl)propan-1-ol (D103, 560 mg, 1.49 mmol) in CH₂Cl₂ (5.00 mL) was added dropwise TFA (1 mL). The reaction solution was stirred at room temperature overnight, concentrated, diluted with CH₂Cl₂ (30 mL), washed with sat. NaHCO₃ (30 mL) and brine (30 mL). The separated organic solution was dried over anhydrous Na₂SO₄ and concentrated. The residue was purified by silica gel column (CH₂Cl₂:MeOH=10:1) to give the title product (245 mg, yield: 56.0%) as a yellow solid.

LC-MS [mobile phase: from 70% water (0.1% TFA) and 30% CH₃CN (0.1% TFA) to 5% water (0.1% TFA) and 95% CH₃CN (0.1% TFA) in 2.0 min]: Rt=1.06 min; MS Calcd: 291.2, MS Found: 292.2 [M+H]⁺.

### Description 105

### 4-(azetidin-1-yl)-6-iodo-2-methoxypyrimidine (D105)

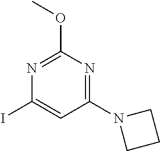

A mixture of 4,6-diiodo-2-methoxypyrimidine (2.00 g, 5.52 mmol), azetidine (0.570 g, 6.07 mmol) and TEA (1.67 g, 16.6 mmol) in DMSO (20.0 mL) was stirred at 60° C. overnight, diluted with H₂O (250 mL), extracted with EtOAc (200 mL×2) and concentrated. The residue was purified by silica gel chromatography column (petroleum ether/EtOAc=8/1) to give the title product (0.640 g, 40.0%) as a white oil.

¹HNMR (300 MHz, CDCl₃): 6.29 (s, 1H), 4.07 (1, J=8.0 Hz, 4H), 3.89 (s, 3H), 2.45-2.38 (m, 2H).

### Description 106

### tert-butyl 4-(1-(6-(azetidin-1-yl)-2-methoxypyrimidin-4-yl)-5-methyl-1H-indazol-6-yl)piperidine-1-carboxylate (D106)

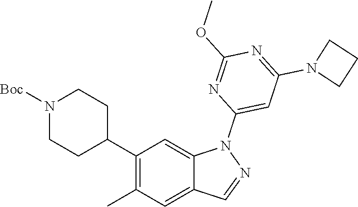 [SMILES: COc1nc(N2CCC2)cc(-n2ncc3cc(C)c(C4CCN(C(=O)OC(C)(C)C)CC4)cc32)n1]

A mixture of tert-butyl 4-(5-methyl-1H-indazol-6-yl)piperidine-1-carboxylate (300 mg, 0.950 mmol), 4-(azetidin-1-yl)-6-iodo-2-methoxypyrimidine (304 mg, 1.05 mmol), N,N′-dimethylcyclohexane-1,2-diamine (27.0 mg, 0.190 mmol), CuI (18.0 mg, 0.0950 mmol) and K₃PO₄ (403 mg, 1.90 mmol) in toluene (4 mL) was stirred at 100° C. for 3 hours, diluted with H₂O (15 mL) and NH₃.H₂O (5 mL) and extracted with EtOAc (20 mL×3). The combined organic layers were concentrated and purified by column chromatography on silica gel (petroleum ether/EtOAc=3/1) to give the crude product (460 mg, 100%) as a yellow oil.

LCMS [column: C₁₈, column size: 4.6×30 mm 5 μm; Dikwa Diamonsil plus; mobile phase: B (CH₃CN), A (0.02% NH₄Ac+5% CH₃CN in water); gradient (B %) in 4 mins. 05-95-POS; flow rate: 1.5 ml/min]: Rt=2.824 min; MS Calcd.: 478, MS Found: 479 [M+H]⁺.

### Description 107

### 1-(6-(azetidin-1-yl)-2-methoxypyrimidin-4-yl)-5-methyl-6-(piperidin-4-yl)-1H-inda-zole (D107)

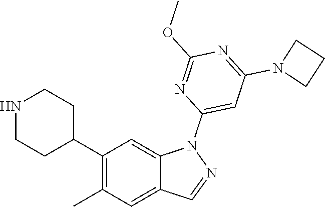 [SMILES: COc1nc(N2CCC2)cc(-n2ncc3cc(C)c(C4CCNCC4)cc32)n1]

A solution of tert-butyl 4-(1-(6-(azetidin-1-yl)-2-methoxypyrimidin-4-yl)-5-methyl-1H-indazol-6-yl)piperidine-1-carboxylate (D106, 460 mg, 0.960 mmol) in DCM (5 mL) and TFA (5 mL) was stirred at room temperature for 1 hour, concentrated, diluted with H₂O (10 mL) and basified with sat. NaHCO₃ to pH=8˜9 and filtered. The filtered cake was dried to give the title product (350 mg, 96.0%) as a yellow solid.

LCMS [column: C₁₈; column size: 4.6×30 mm 5 μm; Dikwa Diamonsil plus; mobile phase: B (CH₃CN), A (0.02% NH₄Ac+5% CH₃CN in water); gradient (B %) in 4 mins. 05-95-POS; flow rate: 1.5 ml/min]: Rt=1.880 min; MS Calcd.: 378, MS Found: 379 [M+H]⁺.

### Description 108

### (R)-tertbutyl 4-(1-(6-(2-(hydroxymethyl)morpholino)-2-methylpyrimidin-4-yl)-5-methyl-1H-indazol-6-yl)piperidine-1-carboxylate (D108)

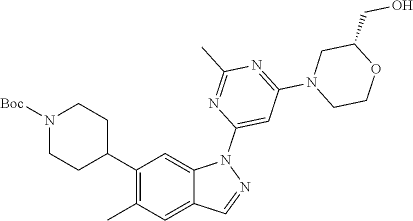 [SMILES: Cc1nc(N2CCO[C@@H](CO)C2)cc(-n2ncc3cc(C)c(C4CCN(C(=O)OC(C)(C)C)CC4)cc32)n1]

A mixture of (R)-(4-(6-iodo-2-methylpyrimidin-4-yl)morpholin-2-yl)methanol (D116, 335 mg, 1.00 mmol), tert-butyl 4-(5-methyl-1H-indazol-6-yl)piperidine-1-carboxylate (D52, 315 mg, 1.00 mmol), CuI (38.0 mg, 0.200 mmol), K₃PO₄ (424 mg, 2.00 mmol) and N,N′-dimethylcyclohexane-1,2-diamine (56.0 mg, 0.400 mmol) in toluene (3 mL) was stirred at 100° C. for 4 hours, diluted with EtOAc (30 mL), washed with NH₃H₂O (15 mL×3), dried over Na₂SO₄, filtered and concentrated. The crude was purified by flash chromatography (petroleum ether/EtOAc=1:1) to give the title product (420 mg, 80.0%) as a white solid.

¹HNMR (400 MHz, CDCl₃): δ 8.75 (s, 1H), 8.06 (s, 1H), 7.51 (s, 1H), 6.95 (s, 1H), 4.38-4.25 (m, 5H), 4.15-4.05 (m, 2H), 3.81-3.65 (m, 5H), 3.15-3.11 (m, 1H), 3.08-2.83 (m, 5H), 2.62 (s, 3H), 2.47 (s, 3H), 1.51 (s, 10H).

### Description 109

### (R)-(4-(2-methyl-6-(5-methyl-6-(piperidin-4-yl)-1H-indazol-1-yl)pyrimidin-4-yl)mo-rpholin-2-yl)methanol (D109)

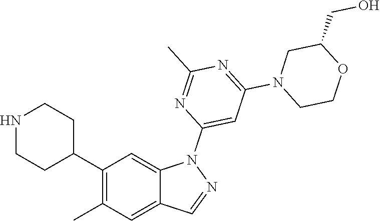 [SMILES: Cc1nc(N2CCO[C@@H](CO)C2)cc(-n2ncc3cc(C)c(C4CCNCC4)cc32)n1]

To a solution of (R)-tert-butyl 4-(1-(6-(2-(hydroxymethyl)morpholino)-2-methylpyri-midin-4-yl)-5-methyl-1H-indazol-6-yl)piperidine-1-carboxylate (D108, 420 mg, 0.800 mmol) in MeOH (2 mL) was added HCl/MeOH (2 M, 1 mL). The mixture was stirred at room temperature for 3 hours, concentrated, dissolved in MeOH (10 mL) and treated with Amberst. The mixture was stirred at room temperature for 30 minutes and filtered. The filtrate was concentrated to give the title product (327 mg, 96%) as a white solid.

¹HNMR (400 MHz, DMSO-d₆): δ 8.75 (s, 1H), 8.34 (s, 1H), 7.65 (s, 1H), 6.99 (s, 1H), 4.89 (s, 1H), 4.36-4.27 (m, 2H), 3.98-3.94 (m, 1H), 3.40-3.56 (m, 9H), 3.34-2.97 (m, 6H), 2.83-2.74 (m, 1H), 2.59 (s, 3H), 2.46 (s, 3H).

### Description 110

### 1-(6-iodo-2-methoxypyrimidin-4-yl)azetidin-3-ol (D110)

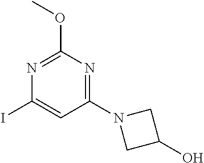

A mixture of 4,6-diiodo-2-methoxypyrimidine (2.00 g, 5.52 mmol), azetidin-3-ol (665 mg, 6.07 mmol) and TEA (1.67 g, 16.6 mmol) in i-PrOH (10 mL) was stirred at 50° C. for 5 hours, diluted with H₂O (20 mL) and extracted with EtOAc (30 mL×2). The combined organic layers were concentrated and purified by silica gel chromatography column (petroleum ether/EtOAc=4/1 to 1/1) to give the title product (1.57 g, 93.0%) as a yellow solid

¹HNMR (300 MHz, CDCl₃): 6.32 (s, 1H), 4.80 (br s, 1H), 3.97-3.92 (m, 4H), 3.89 (s, 3H), 2.68 (s, 1H).

### Description 111

### tert-butyl 4-(1-(6-(3-hydroxyazetidin-1-yl)-2-methoxypyrimidin-4-yl)-5-methyl-1H-indazol-6-yl)piperidine-1-carboxylate (D111)

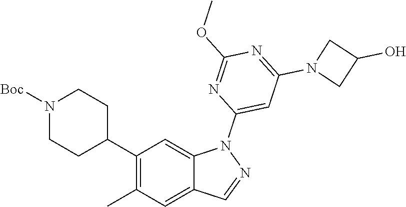 [SMILES: COc1nc(N2CC(O)C2)cc(-n2ncc3cc(C)c(C4CCN(C(=O)OC(C)(C)C)CC4)cc32)n1]

A mixture of tert-butyl 4-(5-methyl-1H-indazol-6-yl)piperidine-1-carboxylate (D52, 500 mg, 1.59 mmol), 1-(6-iodo-2-methoxypyrimidin-4-yl)azetidin-3-ol (D110, 537 mg, 1.75 mmol), N,N′-dimethylcyclohexane-1,2-diamine (114 mg, 0.800 mmol), CuI (61.0 mg, 0.320 mmol) and K₃PO₄ (674 mg, 3.18 mmol) in toluene (4 mL) was stirred at 100° C. for 3 hours, diluted with EtOAc (60 mL) and washed with NH₃.H₂O (20 mL) and brine (20 mL). The organic layer was concentrated and purified by silica gel chromatography column (petroleum ether/EtOAc=2/1 to 1/1) to give the title product (445 mg, 58.0%) as a yellow oil.

¹H-NMR (CDCl₃, 400 MHz): δ 8.71 (s, 1H), 8.06 (s, 1H), 7.52 (s, 1H), 6.47 (s, 1H), 4.82 (br s, 1H), 4.43-4.26 (m, 4H), 4.26-3.92 (m, 5H), 3.01-2.82 (m, 3H), 2.46 (s, 3H), 2.45-2.39 (m, 1H), 1.88-1.85 (m, 2H), 1.71-1.60 (m, 2H), 1.49 (s, 9H).

### Description 112

### 1-(2-methoxy-6-(5-methyl-6-(piperidin-4-yl)-1H-indazol-1-yl)pyrimidin-4-yl)azet-idin-3-ol (D112)

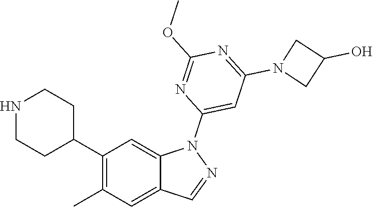 [SMILES: COc1nc(N2CC(O)C2)cc(-n2ncc3cc(C)c(C4CCNCC4)cc32)n1]

To a solution of tert-butyl 4-(1-(6-(3-hydroxyazetidin-1-yl)-2-methoxypyrimidin-4-yl)-5-methyl-1H-indazol-6-yl)piperidine-1-carboxylate (D111, 350 mg, 0.710 mmol) in DCM (4 mL) was added TFA (1 mL). The reaction mixture was stirred at room temperature for 2 hours. adjusted to pH=9˜10 with Sat.NaHCO₃, diluted with H₂O (10 mL) and extracted with DCM (30 mL). The combined organic layers were dried over Na₂SO₄, filtered and concentrated to give the title compound (270 mg, 97%) as a yellow oil.

LCMS [column: C₁₈; column size: 4.6×30 mm 5 μm; Dikwa Diamonsil plus; mobile phase: B (CH₃CN), A (0.02% NH₄Ac+5% CH₃CN in water); gradient (B %) in 4 mins. 10-95-POS; flow rate: 1.5 ml/min]: Rt=1.479 min; MS Calcd.: 394, MS Found: 395 [M+H]⁺.

### Description 113

### (S)-(4-(6-Iodo-2-methylpyrimidin-4-yl)morpholin-2-yl)methanol (D113)

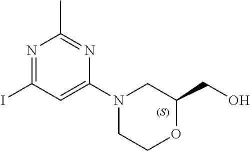

To a solution of (S)-morpholin-2-ylmethanol hydrochloride (430 mg crude, 2.80 mmol) in CH₃OH (5 mL) was added 4,6-diiodo-2-methylpyrimidine (1.10 g, 3.10 mmol) and TEA (850 mg, 8.40 mmol). The resulting mixture was warmed to 60° C. for 2 hrs. TLC showed the reaction was completed. The reaction mixture was diluted with water (20 mL) and extracted EtOAc (20 mL×2). The combined organic layers were concentrated. The crude was purified by gel silico column (PE:EtOAc=5:1) to give the title compound (760 mg, yield 81%) as a white solid.

1H NMR (300 MHz, CDCl₃): δ 6.79 (s, 1H), 4.18-4.01 (m, 3H), 3.79-3.58 (m, 4H), 3.08-2.99 (m, 1H), 2.92-2.84 (m, 1H), 2.46 (s, 3H), 1.97-1.90 (m, 1H).

### Description 114

### 5-methyl-6-(piperidin-4-yl)-1-(tetrahydro-2H-pyran-2-yl)-1H-indazole (D114)

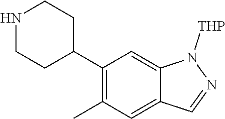

To a suspension of 5-methyl-6-(piperidin-4-yl)-1H-indazole (D51, 10.0 g, 46.5 mmol) and DHP (7.80 g, 93.0 mmol) in THF (200 mL) was added p-TsOH (884 mg, 4.70 mmol) in one portion. The reaction mixture was then stirred at 60° C. overnight. Then the reaction mixture was concentrated, re-dissolved in DCM (1.5 L), washed with sat. NaHCO₃ and brine, dried over Na₂SO₄, filtered and concentrated. The residue was purified by chromatography (MeOH/DCM=1/15) to give the product as a brown solid. (6.70 g, 48.0% yield)

LC-MS [mobile phase: from 95% water (0.1% TFA) and 5% CH₃CN (0.1% TFA) to 5% water (0.1% TFA) and 95% CH₃CN (0.1% TFA) in 2.6 min]: Rt=1.108 min; MS Calcd.: 299.20, MS Found: 300.4 [M+H]⁺.

### Description 115

### (R)-Morpholin-2-ylmethanol hydrochloride (D115)

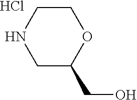

To a solution of (R)-tert-butyl 2-(hydroxymethyl)morpholine-4-carboxylate (500 mg, 2.30 mmol) was added HCl/dioxane (4 M, 10 mL) and stirred for 1 h at rt. TLC showed that the reaction was completed. The reaction was concentrated to give the title compound (420 mg, yield >100%) as a white solid.

1H NMR (300 MHz, DMSO-d6): δ 9.67 (s, 1H), 9.38 (s, 1H), 3.94-3.88 (m, 1H), 3.77-3.67 (m, 2H), 3.45-3.33 (m, 2H), 3.13 (t, J=12.6 Hz, 2H), 2.95-2.87 (m, 1H), 2.78-2.67 (m, 1H).

### Description 116

### (R)-(4-(6-Iodo-2-methylpyrimidin-4-yl)morpholin-2-yl)methanol (D116)

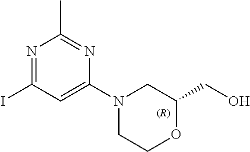

To a solution of (R)-morpholin-2-ylmethanol hydrochloride (423 mg crude, 2.30 mmol) in CH₃OH (10 mL) was added 4,6-diiodo-2-methylpyrimidine (954 mg, 2.75 mmol) and TEA (835 mg, 8.25 mmol). The resulting mixture was warmed to 70° C. and stirred for 2 hrs. LCMS showed that the reaction was completed. The reaction mixture was concentrated to remove solvent, poured into water (40 mL) and extracted with EtOAc (40 mL×2). The combined organic layers were washed with brine, dried over Na₂SO₄ and concentrated. The residue was purified by column (PE:EA=2:1) to give the title compound (639 mg, yield 83%) as a white solid.

1H NMR (300 MHz, CDCl₃): δ 6.79 (s, 1H), 4.22-4.01 (m, 3H), 3.79-3.56 (m, 4H), 3.08-2.98 (m, 1H), 2.88-2.84 (m, 1H), 2.46 (s, 3H), 2.09-2.04 (m, 1H).

### Description 117

### tert-butyl 6-(1-(tert-butoxycarbonyl)piperidin-4-yl)-5-chloro-1H-indazole-1-carboxy-late (D117)

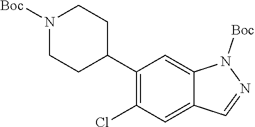 [SMILES: CC(C)(C)OC(=O)N1CCC(c2cc3c(cnn3C(=O)OC(C)(C)C)cc2Cl)CC1]

To a solution of 5-chloro-6-(piperidin-4-yl)-1H-indazole (D82, 2.00 g, 8.49 mmol) and triethylamine (1.72 g, 17.0 mmol) in CH₂Cl₂ (30 mL) was added (Boc)₂O (2.03 g, 9.33 mmol) at rt. The reaction mixture was stirred at rt for 3 h. LC-MS showed the reaction was completed. The reaction mixture was concentrated to dryness and the residue was purified by silica gel chromatography eluted with PE:EtOAc=5:1 to afford the title product as a white solid (1.50 g, yield: 40.0%).

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=2.07 min; MS Calcd: 457.8, MS Found: 458.2 [M+H]⁺.

### Description 118

### tert-butyl 4-(5-chloro-1H-indazol-6-yl)piperidine-1-carboxylate (D118)

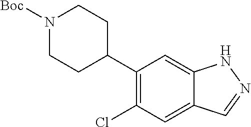 [SMILES: CC(C)(C)OC(=O)N1CCC(c2cc3[nH]ncc3cc2Cl)CC1]

To a solution of tert-butyl 6-(1-(tert-butoxycarbonyl)piperidin-4-yl)-5-chloro-1H-indazole-1-carboxylate (D117, 600 mg, 1.38 mmol) in MeOH (40 mL) was added aq. NaOH (1 M, 40 mL) at rt. The reaction mixture was stirred at rt overnight. LC-MS showed the reaction was completed. The reaction mixture was extracted with CH₂Cl₂ (2×50 mL). The combined organics were washed with brine (50 mL), dried over anhydrous Na₂SO₄ and filtered. The filtrate was concentrated to dryness and the residue was purified by silica gel chromatography eluted with PE:EtOAc (1:1) to afford the title product as a white solid (400 mg, yield: 86.0%).

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=1.57 min; MS Calcd: 335.8, MS Found: 336.1 [M+H]⁺.

### Example 1

### 2-(3-fluoro-4-(1-(2-methoxy-6-morpholinopyrimidin-4-yl)-5-methyl-1H-indazol-6-yl)piperidin-1-yl)ethanol (Single Unknown Isomer 1)

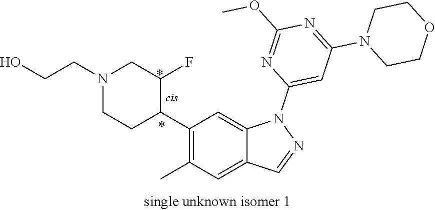 [SMILES: COc1nc(N2CCOCC2)cc(-n2ncc3cc(C)c(C4CCN(CCO)CC4F)cc32)n1]

To a solution of ethyl 2-(3-fluoro-4-(1-(2-methoxy-6-morpholinopyrimidin-4-yl)-5-methyl-1H-indazol-6-yl)piperidin-1-yl)acetate (D27, 90 mg, 0.18 mmol) in THF (2 mL) was added LiAlH₄ (33 mg, 0.88 mmol). The reaction mixture was stirred at RT for 30 min, then quenched with EtOAc and sat. NH₄Cl solution, filtered and concentrated. The purification by C₁₈ flash column (acetonitrile: water=5:95-90:10) to give the title product as a white solid (36 mg, yield: 44%).

¹H NMR (400 MHz, CDCl₃): δ 8.87 (s, 1H), 8.08 (s, 1H), 7.54 (s, 1H), 6.84 (s, 1H), 4.92 (td, J=9.2, 4.8 Hz, 0.5H), 4.80 (td, J=10.0, 4.8 Hz, 0.5H), 4.11 (s, 3H), 3.81˜3.67 (m, 10H), 3.42˜3.39 (m, 1H), 3.18˜3.09 (m, 1H), 3.03 (d, J=10.0 Hz, 1H), 2.74˜2.66 (m, 2H), 2.59˜2.55 (m, 1H), 2.49 (s, 3H), 2.37 (td, J=10.4, 4.4 Hz, 1H), 2.26 (t, J=12.0 Hz, 1H), 2.00˜1.96 (m, 1H), 1.90˜1.80 (m, 1H).

¹⁹F NMR (376 MHz, CDCl₃): δ 183.92 (s)

LC-MS [mobile phase: from 90% water (0.1% FA) and 10% CH₃CN (0.1% FA) to 5% water (0.1% FA) and 95% CH₃CN (0.1% FA) in 9 min, purity 97.59%]: Rt=3.56 min; MS Calcd: 470.2, MS Found: 471.3 [M+H]⁺.

Chiral HPLC (Column: AD Column size: 3×100 mm, 3 μm (Daicel) (UPC). Injection: 10 μl, Mobile phase: CO₂/MeOH/DEA=80/20/0.02, Flow rate: 1.8 ml/min, Wave length: UV 254 nm, Temperature: 35° C.): Rt=2.653 min, ee: 100%

### Example 2

### 2-(3-fluoro-4-(1-(2-methoxy-6-morpholinopyrimidin-4-yl)-5-methyl-1H-indazol-6-yl)piperidin-1-yl)ethanol (Single Unknown Isomer 2)

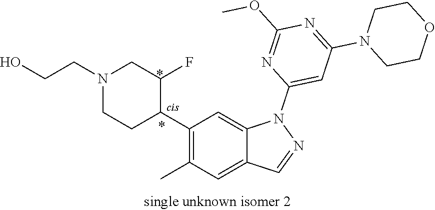 [SMILES: COc1nc(N2CCOCC2)cc(-n2ncc3cc(C)c(C4CCN(CCO)CC4F)cc32)n1]

The title compounds were prepared by a procedure similar to those described for E1 starting from LiAlH₄ and a solution of ethyl 2-(3-fluoro-4-(1-(2-methoxy-6-morpholinop-yrimidin-4-yl) 5-methyl-1H-indazol-6-yl)piperidin-1-yl)acetate in THF.

¹H NMR (400 MHz, CDCl₃): δ 8.87 (s, 1H), 8.07 (s, 1H), 7.54 (s, 1H), 6.85 (s, 1H), 4.92˜4.86 (m, 0.5H), 4.80 (m, 0.5H), 4.11 (s, 3H), 3.81˜3.67 (m, 10H), 3.42˜3.38 (m, 1H), 3.17˜3.08 (m, 1H), 3.03 (d, J=12.0 Hz, 1H), 2.73˜2.66 (m, 2H), 2.58˜2.55 (m, 1H), 2.49 (s, 3H), 2.37˜2.31 (m, 1H), 2.29˜2.23 (m, 1H), 2.01˜1.95 (m, 1H), 1.90˜1.82 (m, 1H).

¹⁹F NMR (376 MHz, CDCl₃): δ 183.92 (s)

LC-MS [mobile phase: from 90% water (0.1% FA) and 10% CH₃CN (0.1% FA) to 5% water (0.1% FA) and 95% CH₃CN (0.1% FA) in 9 min, purity 98.79%]: Rt=3.60 min; MS Calcd: 470.2, MS Found: 471.3 [M+H]⁺.

Chiral HPLC [Column: AD Column size: 3×100 mm, 3 μm (Daicel) (UPC). Injection: 10 μl, Mobile phase: CO₂/MeOH/DEA=80/20/0.02, Flow rate: 1.8 ml/min, Wave length: UV 254 nm, Temperature: 35° C.]: Rt=3.174 min, ee: 100%

### Example 3

### cis-2-(3-fluoro-4-(1-(2-methoxy-6-((R)-3-methylmorpholino)pyrimidin-4-yl)-5-methyl-1H-indazol-6-yl)piperidin-1-yl)ethanol (Single Unknown Isomer 1)

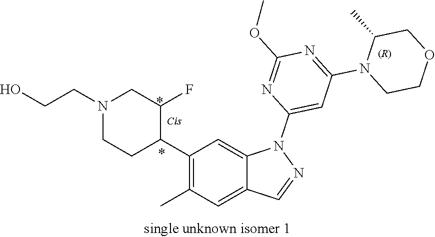

The title compounds were prepared by a procedure similar to those described for E1 starting from a mixture of Ethyl cis-2-(3-fluoro-4-(1-(2-methoxy-6-((R)-3-methylmorpholino) pyrimidin-4-yl)-5-methyl-1H-indazol-6-yl)piperidin-1-yl)acetate (D18) in dried THF, LiAlH₄.

¹H NMR (400 MHz, CDCl₃): δ 8.87 (s, 1H), 8.07 (s, 1H), 7.54 (s, 1H), 6.81 (s, 1H), 4.88˜4.76 (m, 1H), 4.46 (s, 1H), 4.10 (s, 3H), 4.02˜3.99 (d, J=12 Hz, 1H), 3.77˜3.68 (m, 4H), 3.58˜3.56 (m, 1H), 3.42˜3.33 (m, 2H), 3.05˜3.03 (m, 1H), 3.02˜2.99 (m, 1H), 2.69 (s, 2H), 2.56 (s, 1H), 2.48 (s, 3H), 2.02˜1.98 (m, 2H), 2.34˜2.26 (m, 2H), 2.00˜1.98 (m, 1H), 1.86˜1.82 (m, 1H), 1.35˜1.33 (d, J=8 Hz, 3H).

¹⁹F NMR (376.5 MHz, CDCl₃): δ 183.92 (s)

LC-MS [mobile phase: from 80% water (0.1% FA) and 20% CH₃CN (0.1% FA) to 5% water (0.1% FA) and 95% CH₃CN (0.1% FA) in 10.0 min]: Rt=3.96 min; MS Calcd.: 484.6, MS Found: 485.3 [M+H]⁺.

Chiral HPLC [AD 3.0×100 mm, 3 μm (Daicel) (UPC), Phase: CO₂/MeOH/DEA=85/15/0.15, flowrate: 2 mL/min, temperature: 35° C.]: Rt=1.235 min, 96.3% ee.

### Example 4

### cis-2-(3-fluoro-4-(1-(2-methoxy-6-((R)-3-methylmorpholino)pyrimidin-4-yl)-5-methyl-1H-indazol-6-yl)piperidin-1-yl)ethanol (Single Unknown Isomer 2)

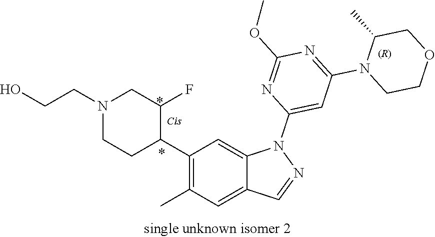

The title compounds were prepared by a procedure similar to those described for E1 starting from 2-(3-fluoro-4-(1-(2-methoxy-6-((R)-3-methylmorpholino) pyrimidin-4-yl)-5-methyl-1H-indazol-6-yl)piperidin-1-yl)acetate (D21).

¹H NMR (400 MHz, CDCl₃): δ 8.87 (s, 1H), 8.07 (s, 1H), 7.54 (s, 1H), 6.8 (s, 1H), 4.89˜4.76 (m, 1H), 4.46 (s, 1H), 4.10 (s, 3H), 4.02˜3.98 (d, J=16.0 Hz, 1H), 3.80˜3.74 (m, 4H), 3.68˜3.62 (m, 1H), 3.42˜3.39 (m, 2H), 3.145˜3.08 (m, 1H), 3.02˜2.99 (m, 1H), 2.71˜2.68 (m, 2H), 2.57 (s, 1H), 2.48 (s, 3H), 2.36˜2.25 (m, 2H), 2.23˜2.00 (m, 1H), 1.97˜1.89 (m, 2H), 1.35˜1.33 (d, J=8.0 Hz, 3H).

¹⁹F NMR (376.5 MHz, CDCl₃): δ 183.92 (s)

LC-MS [mobile phase: from 80% water (0.1% FA) and 20% CH₃CN (0.1% FA) to 5% water (0.1% FA) and 95% CH₃CN (0.1% FA) in 10.0 min]: Rt=4.10 min; MS Calcd.: 484.6, MS Found: 485.3 [M+H]⁺.

Chiral HPLC [AD 3.0×100 mm, 3 μm (Daicel) (UPC), Phase: CO₂/MeOH/DEA=85/15/0.15, flowrate: 2 mL/min, temperature: 35° C.]: Rt=1.381 min, 98.1% ee.

### Examples 5-12

Example 5 to 32 were prepared using a similar procedure of those described for E1.

| Entried | Structures | Analytical data |
|---|---|---|
| E5 | 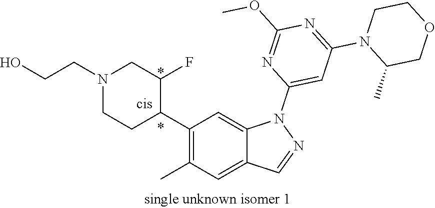 [SMILES: COc1nc(N2CCOC[C@@H]2C)cc(-n2ncc3cc(C)c(C4CCN(CCO)CC4F)cc32)n1] | ¹H NMR (400 MHz, CDCl₃): δ 8.87 (s, 1H), 8.08 (s, 1H), 7.54 (s, 1H), 6.91 (s, 1H), 4.93~4.86 (m, 0.5H), 4.80~4.73 (m, 0.5 H), 4.47~4.43 (m, 1H), 4.11(s,3H), 4.11~4.07 (m, 1H), 4.03 (dd, J = 11.6, 3.6 Hz, 1H), 3.81~3.71 (m, 4H), 3.62 (td, J = 12.0, 2.8 Hz, 1H), 3.42~3.39 (m, 1H), 3.37 (td, J = 13.6, 4.4 Hz, 1H), 3.18~3.10 (m, 1H), 3.03~2.98 (m, 1H), 2.72~2.68 (m, 2H), 2.58 (br, 1H), 2.49 (s, 3H), 2.37 (td, J = 10.4, 3.6 Hz, 1H), 2.30 (td, J = 11.2, 2.0 Hz, 1H), 2.01~1.79 (m, 2H), 1.35 (d, J= 6.8 Hz, 3H), ¹⁹F NMR (376 MHz, CDCl₃): δ 183.93 (s) LC-MS*: purity 100%, Rt = 4.01 min; MS Calcd: 484.3 MS Found: 485.3 [M + H]⁺. Chiral HPLC**: Rt = 2.443 min, ee: 99.16% |
| E6 | 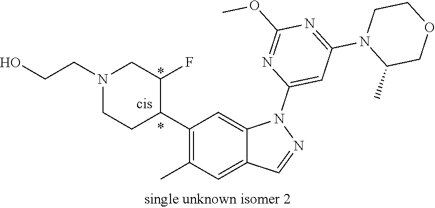 [SMILES: COc1nc(N2CCOC[C@@H]2C)cc(-n2ncc3cc(C)c(C4CCN(CCO)CC4F)cc32)n1] | ¹H NMR (400 MHz, CDCl₃): δ 8.87 (s, 1H), 8.08 (s, 1H), 7.54 (s, 1H), 6.11 (s, 1H), 4.92~4.86 (m, 0.5H), 4.80~4.74 (m, 0.5 H), 4.47~4.43 (m, 1H), 4.11(s,3H), 4.11~4.07 (m, 1H), 4.03 (dd, J = 11.6, 3.6 Hz, 1H), 3.81~3.71 (m, 4H), 3.62 (td, J = 12.4, 3.2 Hz, 1H), 3.42~3.39 (m, 1H), 3.37 (td, J = 12.8, 3.6 Hz, 1H), 3.18~3.10 (m, 1H), 3.03~2.98 (m, 1H), 2.72~2.68 (m, 2H), 2.58 (br, 1H), 2.49 (s, 3H), 2.37 (td, J = 10.0, 4.0 Hz, 1H), 2.30 (td, J = 12.4, 2.4 Hz, 1H), 2.01~1.79 (m, 2H), 1.35 (d, J = 6.8 Hz, 3H), ¹⁹F NMR (376 MHz, CDCl₃): δ 183.92 (s) LC-MS: Rt = 3.69 min; MS Calcd: 484.3, MS Found: 485.3 [M + H]⁺. Chiral HPLC: Rt = 3.143 min, ee: 98.42% |
| E7 | 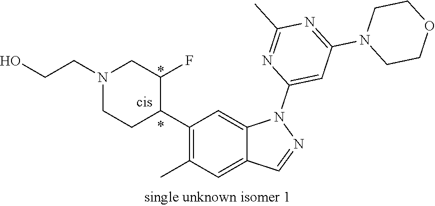 [SMILES: Cc1nc(N2CCOCC2)cc(-n2ncc3cc(C)c(C4CCN(CCO)CC4F)cc32)n1] | ¹H NMR (400 MHz, CDCl₃): δ 8.92 (s, 1H), 8.09 (s, 1H), 7.55 (s, 1H), 6.97 (s, 1H), 4.96~4.82 (m, 1H), 3.84~3.72 (m, 10H), 3.48~3.44 (m, 1H), 3.17~3.04 (m, 2H), 2.75~2.69 (m, 2H), 2.64 (s, 3H), 2.50 (s, 3H), 2.40~2.26 (m, 2H), 2.00~1.86 (m, 2H). ¹⁹F NMR (376 MHz, CDCl₃): δ 183.90 (s) LC-MS***: Rt = 5.20 min; MS Calcd: 454, MS Found: 455.3 [M + H]⁺. Chiral HPLC****: Rt = 7.075 min, ee: 100% |
| E8 | 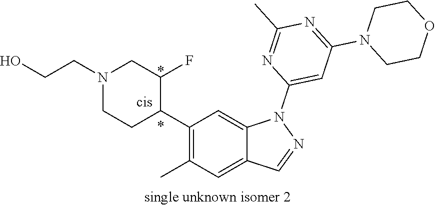 [SMILES: Cc1nc(N2CCOCC2)cc(-n2ncc3cc(C)c(C4CCN(CCO)CC4F)cc32)n1] | ¹H NMR (400 MHz, CDCl₃): δ 8.91 (s, 1H), 8.07 (s, 1H), 7.53 (s, 1H), 6.95 (s. 1H), 4.95~4.80 (m, 1H), 3.82~3.71 (m, 10H), 3.49~3.43 (m, 1H), 3.12~2.99 (m, 2H), 2.72~2.71 (m, 2H), 2.62 (s, 3H), 2.49 (s, 3H), 2.38~2.21 (m, 2H), 2.00~1.86 (m, 2H). ¹⁹F NMR (376 MHz, CDCl₃): δ 183.90 (s) LC-MS: mobile phase: from 80% water (0.1% FA) and 20% CH₃CN (0.1% FA) to 5% water (0.1% FA) and 95% CH3CN (0.1% FA) in 9 min, purity 100%, Rt = 3.25 min; MS Calcd: 454, MS Found: 455.3 [M + H]⁺. Chiral HPLC UPC, Column: AD, 5 μm 0.46 cm I.D. × 25 cm L(Daicel). Injection: 3 μl Mobile phase: CO2/EtOH/ACN/DEA 75/21/9/0.025, Flow rate: 3 mL/min, Wave length: UV 254 nm, Temperature: 35° C., Rt = 5.365 min, ee: 100% |
| E9 | 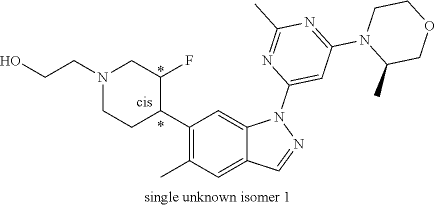 [SMILES: Cc1nc(N2CCOC[C@H]2C)cc(-n2ncc3cc(C)c(C4CCN(CCO)CC4F)cc32)n1] | ¹H NMR (400 MHz, CDCl₃): δ 8.91 (s, 1H), 8.06 (s, 1H), 7.53 (s, 1H), 6.91 (s, 1H), 4.94~4.91 (m, 1H), 4.48 (s, 1H), 4.16~4.13 (m, 1H), 4.02~4.00 (m, 1H), 3.78~3.57 (m, 4H), 3.45~3.44 (m, 1H), 3.32~3.26 (m, 1H), 3.12~3.11 (m, 1H), 3.05~3.03 (m, 1H), 2.71~2.61 (m, 2H), 2.48 (s, 2H), 2.35 (s, 3H), 2.27 (s, 3H), 2.02~1.98 (m, 2H), 2.01~1.88 (m, 3H), 1.35 (s, 3H). ¹⁹F NMR (376.5 MHz, CDCl₃): δ 183.90 (s). LC-MS [mobile phase: 80% water (0.1% FA) and 20% CH₃CN (0.1% FA) in 10.0 min]: Rt = 3.88 min; MS Calcd.:468.5, MS Found: 469.3 [M + H]⁺. Chiral HPLC: AD 4.6 × 250 mm, 3 μm (Daicel) (UPC), Phase: CO₂/MeOH/ACN/DEA = 70/18/12/0.03, flow rate: 2.5 mL/min, temperature: 35° C., Rt = 2.987 min, 99.5% ee. |
| E10 | 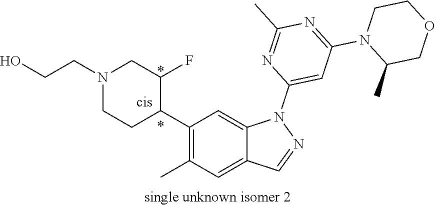 [SMILES: Cc1nc(N2CCOC[C@H]2C)cc(-n2ncc3cc(C)c(C4CCN(CCO)CC4F)cc32)n1] | ¹H NMR (400 MHz, CDCl₃): δ 8.90 (s, 1H), 8.06 (s, 1H), 7.53 (s, 1H), 6.90 (s, 1H), 4.93~4.80 (m, 1H), 4.46 (s, 1H), 4.17~4.10 (m, 1H), 3.78~3.70 (m, 4H), 3.62~3.56 (m, 1H), 3.45~3.42 (m, 1H), 3.32~3.26 (m, 1H), 3.12~3.11 (m, 1H), 3.04~3.02 (m, 1H), 2.71~2.69 (m, 2H), 2.61 (s, 3H), 2.48 (s, 3H), 2.36~2.24 (m, 3H), 1.97~1.88 (m, 2H), 1.33~1.31 (d, J = 8Hz, 3H). ¹⁹F NMR (376.5 MHz, CDCl₃): δ 183.90 (s). LC-MS [mobile phase: 80% water (0.1% FA) and 20% CH₃CN (0.1% FA) in 10.0 min]: Rt = 3.93 min; MS Calcd.:468.5, MS Found: 469.3 [M + H]⁺. Chiral HPLC [AD 4.6 × 250 mm, 3 μm (Daicel) (UPC), Phase: CO2/MeOH/ACN/DEA = 70/18/12/0.03, flow rate: 2.5 mL/min, temperature: 35° C.]: Rt = 3.544 min, 94.94% ee. |
| E11 | 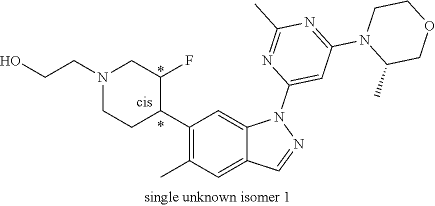 [SMILES: Cc1nc(N2CCOC[C@@H]2C)cc(-n2ncc3cc(C)c(C4CCN(CCO)CC4F)cc32)n1] | ¹H NMR (400 MHz, CDCl₃): δ 8.91 (s, 1H), 8.07 (s, 1H), 7.53 (s, 1H), 6.91 (s, 1H), 4.97~4.90 (m, 0.5H), 4.85~4.79 (m, 0.5 H), 4.47~4.45 (m, 1H), 4.19~4.14 (m, 1H), 4.04~4.00 (m, 1H), 3.81~3.71 (m, 4H), 3.63 (td, J= 11.6, 2.4 Hz, 1H), 3.46~3.43 (m, 1H), 3.33 (td, J = 12.8, 3.6 Hz, 1H), 3.18~3.03 (m, 2H), 2.73~2.72 (m, 2H), 2.62 (s, 3H), 2.49 (s, 3H), 2.38~2.25 (m, 2H), 1.98~1.86 (m, 2H), 1.33 (d, J = 6.8 Hz, 3H), ¹⁹F NMR (376 MHz, CDCl₃): δ 183.92 (s) LC-MS*: Rt = 4.02 min; MS Calcd: 468.3, MS Found: 469.3 [M + H]⁺. Chiral HPLC [Column: AD Column size: 4.6 × 250 mm, 5 μm (Daicel) (UPC). Injection: 10 μl Mobile phase: CO₂/MeOH/ACN/DEA = 75/15/10/0.025, Flow rate: 2.5 ml/min, Wave length: UV 254 nm, Temperature: 35° C.]: Rt = 4.510 min, ee: 100% |
| E12 | 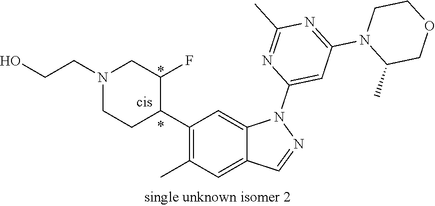 [SMILES: Cc1nc(N2CCOC[C@@H]2C)cc(-n2ncc3cc(C)c(C4CCN(CCO)CC4F)cc32)n1] | ¹H NMR (400 MHz, CDCl₃): δ 8.90 (s, 1H), 8.06 (s, 1H), 7.53 (s, 1H), 6.90 (s, 1H), 4.93~4.80 (m, 1H), 4.46 (s, 1H), 4.17~4.10 (m, 1H), 3.78~3.70 (m, 4H), 3.62~3.56 (m, 1H), 3.45~3.42 (m, 1H), 3.32~3.26 (m, 1H), 3.12~3.11 (m, 1H), 3.04~3.02 (m, 1H), 2.71~2.69 (m, 2H), 2.61 (s, 3H), 2.48 (s, 3H), 2.36~2.24 (m, 3H), 1.97~1.88 (m, 2H), 1.33~1.31 (d, J = 8Hz, 3H). ¹⁹F NMR (376.5 MHz, CDCl₃): δ 183.90 (s). LC-MS [mobile phase: 80% water (0.1% FA) and 20% CH₃CN (0.1% FA) in 10.0 min]: Rt = 3.93 min; MS Calcd.:468.5, MS Found: 469.3 [M + H]⁺. Chiral HPLC [AD 4.6 × 250 mm, 3 μm (Daicel) (UPC), Phase: CO₂/MeOH/ACN/DEA = 70/18/12/0.03, flow rate: 2.5 mL/min, temperature: 35° C.]: Rt = 3.544 min, 94.94% ee. |

*mobile phase: from 90% water (0.1% FA) and 10% CH₃CN (0.1% FA) to 5% water (0.1% FA) and 95% CH₃CN (0.1% FA) in 9 min,
**Chiral method: Column: AD Column size: 3 × 100 mm, 3 μm (Daicel) (UPC). Injection: 10 μl Mobile phase: CO/MeOH/DEA = 80/20/0.02, Flow rate: 1.8 ml/min, Wave length: UV 254 nm, Temperature: 35° C.,
***mobile phase: from 50% water (0.1% NH₃OH) and 20% CH₃CN (0.1% NH₃OH) to 5% water (0.1% NH₃OH) and 95% CH₃CN (0.1% NH₃OH) in 9 min.
****UPC, Column: AD, 5 μm, 0.46 cm I.D. × 25 cm L. Injection: 3 μl Mobile phase: CO₂/EtOH/ACN/DEA 75/21/9/0.025, Flow rate: 3 mL/min, Wave length: UV 254 nm, Temperature: 35° C.,

### Examples 13 to 16

### 1-(4-(6-(6-(1-(2-fluoroethyl)piperidin-4-yl)-5-methyl-1H-indazol-1-yl)-2-methoxypyrimidin-4-yl)morpholin-2-yl)ethanol (Single Unknown Isomer 1; Single Unknown Isomer 2; Single Unknown Isomer 3; Single Unknown Isomer 4)

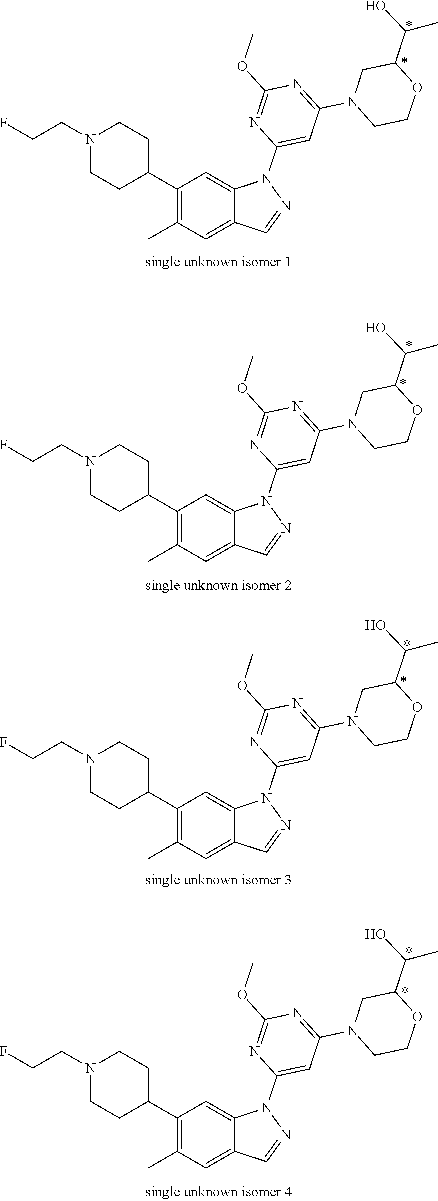 [SMILES: COc1nc(N2CCOC(C(C)O)C2)cc(-n2ncc3cc(C)c(C4CCN(CCF)CC4)cc32)n1 ; COc1nc(N2CCOC(C(C)O)C2)cc(-n2ncc3cc(C)c(C4CCN(CCF)CC4)cc32)n1 ; COc1nc(N2CCOC(C(C)O)C2)cc(-n2ncc3cc(C)c(C4CCN(CCF)CC4)cc32)n1 ; COc1nc(N2CCOC(C(C)O)C2)cc(-n2ncc3cc(C)c(C4CCN(CCF)CC4)cc32)n1]

To a solution of 1-(4-(6-(6-(1-(2-fluoroethyl)piperidin-4-yl)-5-methyl-1H-indazol-1-yl)-2-methoxypyrimidin-4-yl)morpholin-2-yl)ethanone (257 mg, 0.520 mmol) in MeOH (30 mL) was added NaBH₄ (39.0 mg, 1.04 mmol) at 0° C. The reaction mixture was stirred at rt for 3 h. LCMS showed reaction was completed. The reaction mixture was diluted with CH₂Cl₂ (20 mL) and H₂O (20 mL). The separated aqueous layer was extracted with CH₂Cl₂ (2×30 mL) and the combined organic layers were washed with water, brine, dried over anhydrous Na₂SO₄, filtered and concentrated to give the crude product as a white solid (254 mg, yield: 98%).

LC-MS [mobile phase: from 50% water (0.1% FA) and 50% CH₃CN (0.1% FA) to 5% water (0.1% FA) and 95% CH₃CN (0.1% FA) in 2.6 min]: Rt=0.79 min; MS Calcd: 498.6, MS Found: 499.2 [M+H]⁺.

### Chiral Separation:

### Method:

Column: AD-H; Column size: 0.46 cm I.D.×15 cm L; Injection: 2 ul; Mobile phase:CO₂:EtOH (0.05% NH₃.H₂O)=60:40; Flow rate: 0.5 ml; Wave length: UV 254 nm; Temperature: 25° C.; Sample solution in EtOH

### Single Unknown Isomer 1

¹H NMR (400 MHz, CDCl₃) δ 8.74 (s, 1H), 8.07 (s, 1H), 7.51 (s, 1H), 6.85 (s, 1H), 4.70˜4.68 (m, 1H), 4.58˜4.56 (m, 1H), 4.37˜4.24 (m, 2H), 4.14 (s, 3H), 4.02˜4.01 (d, J=2.0 Hz, 1H), 3.95˜3.94 (m, 1H), 3.69˜3.68 (m, 1H), 3.47˜3.42 (m, 1H), 3.15˜3.01 (m, 4H), 2.85˜2.74 (m, 3H), 2.46 (s, 3H), 2.32˜2.25 (m, 2H), 2.10 (s, 1H), 1.92˜1.90 (m, 4H), 1.30˜1.28 (d, J=8.0 Hz, 3H).

¹⁹F NMR (376 MHz, CDCl₃): δ 217.725 (s).

LC-MS [mobile phase: from 95% water (0.1% FA) and 5% CH₃CN (0.1% FA) to 5% water (0.1% FA) and 95% CH₃CN (0.1% FA) in 10 min]: Purity 100%; Rt=4.66 min; MS Calcd: 498.6, MS Found: 499.3 [M+H]⁺.

### Single Unknown Isomer 2

¹H NMR (400 MHz, CDCl₃) δ 8.74 (s, 1H), 8.07 (s, 1H), 7.51 (s, 1H), 6.85 (s, 1H), 4.70˜4.68 (m, 1H), 4.59˜4.56 (m, 1H), 4.37˜4.26 (m, 2H), 4.14 (s, 3H), 4.05˜4.02 (d, J=12.0 Hz, 1H), 3.97˜3.93 (m, 1H), 3.72˜3.66 (m, 1H), 3.46˜3.43 (m, 1H), 3.16˜3.02 (m, 4H), 2.83˜2.75 (m, 3H), 2.46 (s, 3H), 2.32˜2.26 (m, 2H), 2.10 (s, 1H), 1.92˜1.90 (m, 4H), 1.30˜1.28 (d, J=8.0 Hz, 3H).

¹⁹F NMR (376 MHz, CDCl₃): δ217.711 (s).

LC-MS [mobile phase: from 95% water (0.1% FA) and 5% CH₃CN (0.1% FA) to 5% water (0.1% FA) and 95% CH₃CN (0.1% FA) in 10 min]: Purity 98.59%; Rt=4.65 min; MS Calcd: 498.6, MS Found: 499.3 [M+H]⁺.

### Single Unknown Isomer 3

¹H NMR (400 MHz, CDCl₃) δ 8.75 (s, 1H), 8.07 (s, 1H), 7.51 (s, 1H), 6.84 (s, 1H), 4.70˜4.68 (m, 1H), 4.58˜4.56 (m, 1H), 4.34˜4.22 (m, 2H), 4.14 (s, 3H), 4.08˜4.06 (m, 1H), 3.79˜3.76 (m, 1H), 3.70˜3.65 (m, 1H), 3.36˜3.32 (m, 1H), 3.16˜3.09 (m, 3H), 2.94˜2.74 (m, 4H), 2.46 (s, 4H), 2.32˜2.26 (m, 2H), 1.92˜1.90 (m, 4H), 1.30˜1.28 (d, J=8.0 Hz, 3H).

¹⁹F NMR (376 MHz, CDCl₃):δ217.711 (s).

LC-MS [mobile phase: from 95% water (0.1% FA) and 5% CH₃CN (0.1% FA) to 5% water (0.1% FA) and 95% CH₃CN (0.1% FA) in 10 min]: Purity 97.14%; Rt=4.55 min; MS Calcd: 498.6, MS Found: 499.3 [M+H]⁺.

### Single Unknown Isomer 4

¹H NMR (400 MHz, CDCl₃) δ 8.75 (s, 1H), 8.07 (s, 1H), 7.51 (s, 1H), 6.84 (s, 1H), 4.70˜4.67 (m, 1H), 4.58˜4.56 (m, 1H), 4.34˜4.22 (m, 2H), 4.14 (s, 3H), 4.09˜4.06 (m, 1H), 3.79˜3.76 (m, 1H), 3.71˜3.64 (m, 1H), 3.36˜3.31 (m, 1H), 3.16˜3.08 (m, 3H), 2.83˜2.74 (m, 4H), 2.46 (s, 4H), 2.32˜2.26 (m, 2H), 1.92˜1.90 (m, 4H), 1.29˜1.28 (d, J=4.0 Hz, 3H).

¹⁹F NMR (376 MHz, CDCl₃):δ217.732 (s).

LC-MS [mobile phase: from 95% water (0.1% FA) and 5% CH₃CN (0.1% FA) to 5% water (0.1% FA) and 95% CH₃CN (0.1% FA) in 10 min]: Purity 98.59%; Rt=4.52 min; MS Calcd: 498.6, MS Found: 499.3 [M+H]⁺.

### Example 17

### 1-(6-(6-((3S,4R)-3-fluoro-1-(2-fluoroethyl)piperidin-4-yl)-5-methyl-1H-indazol-1-yl)-2-methylpyrimidin-4-yl)azetidin-3-ol (Single Cis Isomer 1)

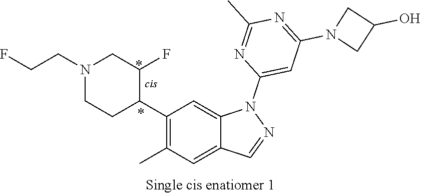 [SMILES: Cc1nc(N2CC(O)C2)cc(-n2ncc3cc(C)c(C4CCN(CCF)CC4F)cc32)n1]

To a solution of 6-((3S,4R)-3-fluoro-1-(2-fluoroethyl)piperidin-4-yl)-5-methyl-1-(2-methyl-6-(3-((tetrahydro-2H-pyran-2-yl)oxy)azetidin-1-yl)pyrimidin-4-yl)-1H-indazole (D62: 40 mg, 0.08 mmol) in DCM (6 mL) was added TFA (1 mL) at 0° C. The reaction mixture was warmed to room temperature and stirred overnight. The solvent and TFA was removed under vacuum and purified by pre-HPLC to give the title product (8.5 mg, yield 25.3%) as an ivory solid.

¹H NMR (400 MHz, CDCl₃): δ 8.90 (s, 1H), 8.07 (s, 1H), 7.52 (s, 1H), 6.60 (s, 1H), 4.84 (m, 2H), 4.69-4.71 (t, J=4.4 Hz, 1H), 4.57-4.59 (t, J=4.4 Hz, 1H), 4.40-4.44 (t, J=7.6 Hz, 2H), 4.00-4.02 (m, 1H), 3.44-3.48 (m, 1H), 3.05-3.07 (d, 2H), 2.83-2.90 (m, 2H), 2.64 (s, 1H), 2.48 (s, 1H), 2.30-2.37 (m, 2H), 1.93-1.95 (m, 2H). ¹⁹F NMR (400 MHz, CDCl₃): δ −181.7 (s, 1F), −216.8 (s, 1F).

LC-MS [mobile phase: from 90% water (0.1% TFA) and 10% CH₃CN (0.1% TFA) to 5% water (0.1% TFA) and 95% CH₃CN (0.1% TFA) in 10 min, purity 95.9%]: Rt=5.15 min; MS Calcd.: 442.23, MS Found: 443.7 [M+H]⁺.

### Example 18

1-(6-(6-((3S,4R)-3-fluoro-1-(2-fluoroethyl)piperidin-4-yl)-5-methyl-1H-indazol-1-yl)-2-methylpyrimidin-4-yl)azetidin-3-ol (Single Cis Enatiomer 2)

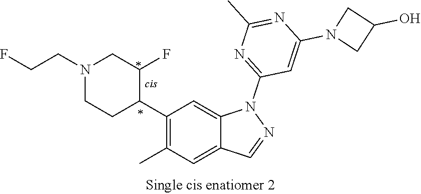 [SMILES: Cc1nc(N2CC(O)C2)cc(-n2ncc3cc(C)c(C4CCN(CCF)CC4F)cc32)n1]

The title compound was prepared by a procedure similar to those described for E17 starting from a solution of 6-((3R,4S)-3-fluoro-1-(2-fluoroethyl)piperidin-4-yl)-5-methyl-1-(2-methyl-6-(3-((tetrahydro-2H-pyran-2-yl)oxy)azetidin-1-yl)pyrimidin-4-yl)-1H-indazole (D63) in DCM and TFA.

¹H NMR (400 MHz, CDCl₃): δ 8.90 (s, 1H), 8.07 (s, 1H), 7.52 (s, 1H), 6.60 (s, 1H), 4.83-4.84 (m, 2H), 4.70 (m, 1H), 4.58 (m, 1H), 4.40-4.44 (t, J=7.6 Hz, 2H), 3.99-4.02 (m, 1H), 3.44-3.48 (m, 1H), 3.05-3.07 (d, 2H), 2.83-2.90 (m, 2H), 2.64 (s, 1H), 2.48 (s, 1H), 2.30-2.37 (m, 2H), 1.93-1.95 (m, 2H).

¹⁹F NMR (400 MHz, CDCl₃): δ −181.789 (s, 1F), −216.896 (d, J=6.0 Hz, 1F).

LC-MS [mobile phase: from 80% water (0.1% TFA) and 20% CH₃CN (0.1% TFA) to 5% water (0.1% TFA) and 95% CH₃CN (0.1% TFA) in 10 min, purity 100%]: Rt=2.62 min; MS Calcd.: 442.23, MS Found: 443.7 [M+H]⁺.

### Example 19

### (R)-2-(4-(5-methyl-1-(2-methyl-6-(3-methylmorpholino)pyrimidin-4-yl)-1H-indazol-6-yl)piperidin-1-yl)ethanol

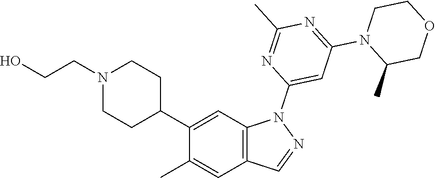 [SMILES: Cc1nc(N2CCOC[C@H]2C)cc(-n2ncc3cc(C)c(C4CCN(CCO)CC4)cc32)n1]

The title compound was prepared by a procedure similar to the described for E1 starting from a solution of (R)-ethyl 2-(4-(5-methyl-1-(2-methyl-6-(3-methylmorpholino)pyrimi-din-4-yl)-1H-indazol-6-yl)piperidin-1-yl)acetate in dry THF and LiAlH₄.

¹H NMR (400 MHz, CDCl₃): δ 8.81 (s, 1H), 8.04 (s, 1H), 7.50 (s, 1H), 6.90 (s, 1H), 4.47˜4.46 (m, 1H), 4.16˜4.13 (d, J=13.2 Hz, 1H), 4.02˜3.99 (m, 1H), 3.81˜3.73 (m, 2H), 3.68 (t, J=5.6 Hz, 2H), 3.63 (td, J=12.0, 2.8 Hz, 1H), 3.32 (td, J=12.8, 3.6 Hz, 1H), 3.14 (d, J=11.6 Hz, 2H), 2.88˜2.84 (m, 2H), 2.65˜2.64 (m, 1H), 2.63 (s, 3H), 2.46 (s, 3H), 2.29˜2.27 (m, 2H), 1.93˜1.72 (m, 4H), 1.33 (d, J=6.8 Hz, 3H).

LC-MS [mobile phase: 70% water (0.1% FA) and 30% CH₃CN (0.1% FA) in 10.0 min]: Rt=4.12 min; MS Calcd.: 450.6, MS Found: 451.4 [M+H]⁺.

### Example 20

### (R)-2-(4-(1-(2-methoxy-6-(3-methylmorpholino)pyrimidin-4-yl)-5-methyl-1H-indazol-6-yl)piperidin-1-yl)ethanol

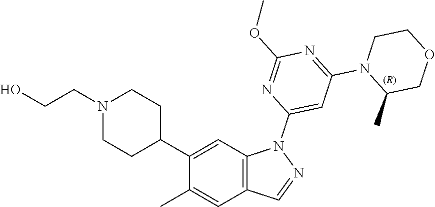

The title compound was prepared by a procedure similar to the described for E1 starting from LiAlH₄ and a solution of (R)-ethyl 2-(4-(1-(2-methoxy-6-(3-methylmor-pholino)pyrimidin-4-yl)-5-methyl-1H-indazol-6-yl)piperidin-1-yl)acetatein THF.

¹H NMR (400 MHz, CDCl₃): δ 8.77 (s, 1H), 8.06 (s, 1H), 7.51 (s, 1H), 7.81 (s, 1H), 4.47 (br, 1H), 4.14 (s, 3H), 4.14˜4.07 (m, 1H), 4.02˜3.99 (m, 1H), 3.78˜3.73 (m, 2HO, 3.68˜3.64 (m, 2H), 3.61˜3.55 (m, 1H), 3.37˜3.29 (m, 1H), 3.10 (d, J=11.6 Hz, 2H), 2.86 (m, 1H), 2.64˜2.60 (m, 2H), 2.46 (s, 3H), 2.28˜2.26 (m, 2H), 1.90˜1.82 (m, 4H), 1.34 (d, J=6.8 Hz, 3H).

LC-MS [mobile phase: from 90% water (0.1% FA) and 10% CH₃CN (0.1% FA) to 5% water (0.1% FA) and 95% CH₃CN (0.1% FA) in 10 min, purity 96.9%]: Rt=4.85 min; MS Calcd: 466.6, MS Found: 467.4 [M+H]⁺.

### Example 21

### (S)-2-(4-(1-(2-methoxy-6-(3-methylmorpholino)pyrimidin-4-yl)-5-methyl-1H-indazol-6-yl)piperidin-1-yl)ethanol

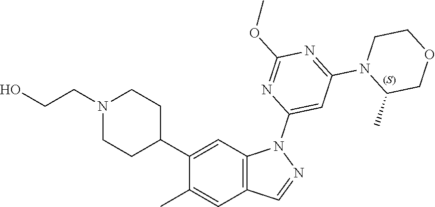

The title compound was prepared by a procedure similar to the described for E1 starting from a solution of (S)-ethyl 2-(4-(1-(2-methoxy-6-(3-methylmorpholino)pyrimidin-4-yl)-5-methyl-1H-indazol-6-yl)piperidin-1-yl)acetate in THF and LiAlH₄.

¹H NMR (400 MHz, CDCl₃): δ 8.77 (s, 1H), 8.06 (s, 1H), 7.51 (s, 1H), 6.81 (s, 1H), 4.47˜4.45 (m, 1H), 4.14 (s, 3H), 4.11 (d, J=13.6 Hz, 1H), 4.02 (dd, J=11.2, 3.2 Hz, 1H), 3.81˜3.71 (m, 2H), 3.65 (t, J=5.2 Hz, 2H), 3.62 (td, J=12.0, 2.8 Hz, 1H), 3.36 (td, J=13.2, 4.0 Hz, 1H), 3.11 (d, J=11.2 Hz, 2H), 2.89˜2.83 (m, 1H), 2.61 (t, J=5.2 Hz, 2H), 2.46 (s, 3H), 2.30˜2.24 (m, 2H), 1.93˜1.72 (m, 4H), 1.35 (d, J=6.8 Hz, 3H).

LC-MS [mobile phase: from 90% water (0.1% FA) and 10% CH₃CN (0.1% FA) to 5% water (0.1% FA) and 95% CH₃CN (0.1% FA) in 9 min, purity 98.9%]: Rt=4.96 min; MS Calcd: 466.3, MS Found: 467.4 [M+H]⁺.

### Example 22

### 2-(4-(1-(6-(azetidin-1-yl)-2-methoxypyrimidin-4-yl)-5-methyl-1H-indazol-6-yl)-3-fluoropiperidin-1-yl)ethanol (Single Unknown Isomer, Rt=3.381 Min)

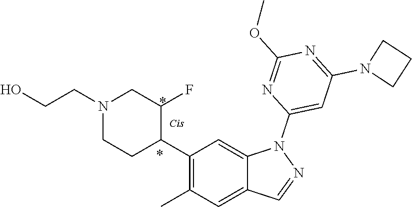 [SMILES: COc1nc(N2CCC2)cc(-n2ncc3cc(C)c(C4CCN(CCO)CC4F)cc32)n1]

To a solution of ethyl 2-(4-(1-(6-(azetidin-1-yl)-2-methoxypyrimidin-4-yl)-5-methyl-1H-indazol-6-yl)-3-fluoropiperidin-1-yl)acetate (D76, 49 mg, 0.10 mmol) in THF (5 mL) was added LiAlH₄ (19 mg, 0.51 mmol). The reaction mixture was stirred at rt for 60 min., quenched with EtOAc and sat. NH₄Cl and filtered. The filtrate was concentrated and purified by C₁₈ flash column (acetonitrile:water=5:95˜95:5) to give the title product (18.0 mg, yield: 40.0%) as a white solid.

¹H NMR (400 MHz, CDCl₃): δ 8.89 (s, 1H), 8.08 (s, 1H), 7.54 (s, 1H), 6.43 (s, 1H), 4.90 (td, J=9.6, 4.4 Hz, 0.5H), 4.78 (td, J=9.6, 4.4 Hz, 0.5H), 4.18 (t, J=7.6 Hz, 4H), 4.11 (s, 3H), 3.68 (s, 2H), 3.41˜3.38 (m, 1H), 3.18˜3.08 (m, 1H), 3.02 (d, J=11.6 Hz, 1H), 2.73˜2.64 (m, 2H), 2.54˜2.51 (m, 1H), 2.48 (s, 3H), 2.48˜2.39 (m, 2H), 2.37 (td, J=10.0, 4.4 Hz, 1H), 2.29 (td, J=11.6, 2.4 Hz, 1H), 2.02˜1.96 (m, 1H), 1.88˜1.77 (m, 1H).

¹⁹F NMR (376 MHz, CDCl₃): δ 183.92 (s)

LC-MS [mobile phase: from 90% water (0.1% TFA) and 10% CH₃CN (0.1% T FA) to 5% water (0.1% TFA) and 95% CH₃CN (0.1% TFA) in 9 min]: Rt=4.02 min; MS Calcd: 440.2, MS Found: 441.3 [M+H]⁺.

Chiral HPLC [method: Column: AD Column size: 3×100 mm, 3 μm (Daicel) (UPC). Injection: 10 μl, Mobile phase: CO₂/MeOH/DEA=80/20/0.02, Flow rate: 1.8 ml/min, Wave length: UV 254 nm, Temperature: 35° C.]: Rt=3.381 min, ee: 99.92%

### Example 23

### 2-(4-(1-(6-(azetidin-1-yl)-2-methoxypyrimidin-4-yl)-5-methyl-1H-indazol-6-yl)-3-fluoropiperidin-1-yl)ethanol (Single Unknown Isomer, Rt=3.381 Min)

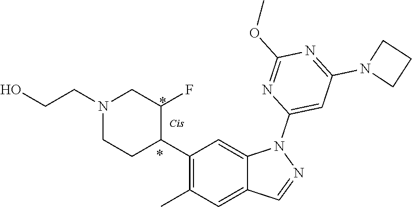 [SMILES: COc1nc(N2CCC2)cc(-n2ncc3cc(C)c(C4CCN(CCO)CC4F)cc32)n1]

The title compound was prepared by a procedure similar to that described for E22 starting from LiAlH₄ and a solution of ethyl 2-(4-(1-(6-(azetidin-1-yl)-2-methoxypyrimidin-4-yl)-5-methyl-1H-indazol-6-yl)-3-fluoropiperidin-1-yl)acetate (D79) in THF.

¹H NMR (400 MHz, CDCl₃): δ 8.89 (s, 1H), 8.08 (s, 1H), 7.54 (s, 1H), 6.44 (s, 1H), 4.90 (td, J=10.0, 4.4 Hz, 0.5H), 4.78 (td, J=10.0, 4.4 Hz, 0.5H), 4.18 (t, J=7.2 Hz, 4H), 4.11 (s, 3H), 3.68 (s, 2H), 3.41˜3.38 (m, 1H), 3.18˜3.09 (m, 1H), 3.02 (d, J=10.8 Hz, 1H), 2.74˜2.64 (m, 2H), 2.54˜2.51 (m, 1H), 2.48 (s, 3H), 2.48˜2.38 (m, 2H), 2.37 (td, J=10.4, 4.0 Hz, 1H), 2.29 (td, J=12.0, 2.4 Hz, 1H), 2.00˜1.94 (m, 1H), 1.87˜1.78 (m, 1H).

¹⁹F NMR (376 MHz, CDCl₃): δ 183.92 (s)

LC-MS [mobile phase: from 90% water (0.1% TFA) and 10% CH₃CN (0.1% TFA) to 5% water (0.1% TFA) and 95% CH₃CN (0.1% TFA) in 9 min]: Rt=4.00 min; MS Calcd: 440.2, MS Found: 441.3 [M+H]⁺.

Chiral HPLC [method: Column: AD Column size: 3 mm×100 mm, 3 μm (Daicel) (UPC). Injection: 10 μl, Mobile phase: CO₂/MeOH/DEA=80/20/0.02, Flow rate: 1.8 ml/min, Wave length: UV 254 nm, Temperature: 35° C.]: Rt=4.868 min, ee: 97.22%

### Examples 24 and 25

### 1-(4-(5-chloro-1-(6-((R)-2-(hydroxymethyl)morpholino)-2-methylpyrimidin-4-yl)-1H-indazol-6-yl)piperidin-1-yl)propan-2-ol (Single Unknown Isomer 1, Rt=4.323 Min; Single Unknown Isomer 2, Rt=4.550 Min)

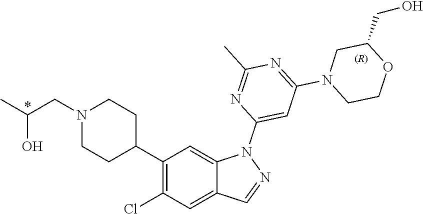

The mixture (D85) was chirally separated by SFC to give the below isomers.

### Chiral Separation:

Method: Column: AD-H; Column size: 0.46 cm×15 cm; Mobile phase: CO₂:IPA (0.1% NH₃:H₂O)=70:30; Flow rate: 0.5 ml; Wave length: UV 254 nm; Temperature: 25° C.; Sample in EtOH

### Peak 1 (E24): Single Unknown Isomer 1

¹H NMR (400 MHz, CDCl₃): δ 8.91 (s, 1H), 8.07 (s, 1H), 7.74 (s, 1H), 6.96 (s, 1H), 4.32˜4.28 (m, 2H), 4.08˜4.05 (m, 1H), 3.88 (s, 1H), 3.78˜3.74 (m, 1H), 3.71˜3.68 (m, 3H), 3.23˜3.09 (m, 3H), 3.01˜2.92 (m, 2H), 2.63 (s, 3H), 2.56˜2.50 (m, 1H), 2.41˜2.39 (m, 1H), 2.38˜2.34 (m, 1H), 2.16˜2.13 (m, 1H), 2.04˜2.01 (m, 2H), 1.91˜1.75 (m, 3H), 1.18 (s, 1H), 1.16 (s, 3H).

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=0.77 min; MS Calcd: 500.2, MS Found: 501.2 [M+H]⁺.

Chiral HPLC: Rt=4.323 min, ee: 100%

### Peak 2 (E25): Single Unknown Isomer 2

¹H NMR (400 MHz, CDCl₃): δ 8.90 (s, 1H), 8.07 (s, 1H), 7.74 (s, 1H), 6.96 (s, 1H), 4.30 (s, 2H), 4.09˜4.05 (m, 1H), 3.89 (s, 1H), 3.78˜3.74 (m, 1H), 3.71˜3.68 (m, 3H), 3.22˜3.11 (m, 3H), 2.98˜2.96 (m, 2H), 2.63 (s, 3H), 2.52 (s, 1H), 2.38˜2.37 (m, 1H), 2.30 (s, 1H), 2.15 (s, 1H), 2.04˜2.01 (m, 2H), 1.87˜1.78 (m, 3H), 1.17 (s, 1H), 1.16 (s, 3H).

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=0.78 min; MS Calcd: 500.2, MS Found: 501.2 [M+H]⁺.

Chiral HPLC: Rt=4.550 min, ee: 98.1%

### Examples 26 and 27

### 1-(4-(5-chloro-1-(6-((S)-2-(hydroxymethyl)morpholino)-2-methylpyrimidin-4-yl)-1H-indazol-6-yl)piperidin-1-yl)propan-2-ol (Single Unknown Isomer 1, Rt=4.203 Min; Single Unknown Isomer 2, Rt=4.413 Min)

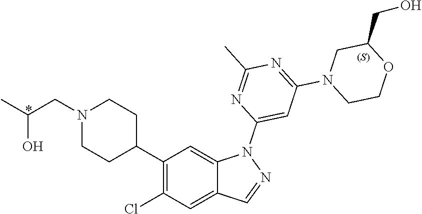

The title compounds were prepared by a procedure similar to that described for E24 and E25 starting from a solution of (S)-1-(4-(5-chloro-1-(6-(2-(hydroxymethyl)morpholino)-2-methylpyrimidin-4-yl)-1H-indazol-6-yl)piperidin-1-yl)propan-2-one (D86) in MeOH and NaBH₄ at 0° C. for 3 h.

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=0.77 min; MS Calcd: 500.2, MS Found: 501.2 [M+H]⁺.

### Chiral Separation:

Method: Column: AD-H; Column size: 0.46 cm×15 cm Mobile phase: CO₂:IPA (0.1% NH₃.H₂O)=70:30; Flow rate: 0.5 ml; Wave length: UV 254 nm; Temperature: 25° C.; Sample in EtOH

### Peak 1 (E26): Single Unknown Isomer 1

¹H NMR (400 MHz, CDCl₃): δ 8.90 (s, 1H), 8.07 (s, 1H), 7.74 (s, 1H), 6.96 (s, 1H), 4.30 (s, 2H), 4.08˜4.05 (m, 1H), 3.89 (s, 1H), 3.80˜3.74 (m, 1H), 3.71˜3.68 (m, 3H), 3.22˜3.11 (m, 3H), 3.00˜2.95 (m, 2H), 2.63 (s, 3H), 2.52 (s, 1H), 2.40˜2.37 (m, 1H), 2.33˜2.30 (m, 1H), 2.15 (s, 1H), 2.04˜2.01 (m, 2H), 1.88˜1.75 (m, 3H), 1.17 (s, 1H), 1.16 (s, 3H).

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=0.78 min; MS Calcd: 500.2, MS Found: 501.2 [M+H]⁺.

Chiral HPLC: Rt=4.203 min, ee: 100%

### Peak 2 (E27): Single Unknown Isomer 2

¹H NMR (400 MHz, CDCl3): δ 8.90 (s, 1H), 8.07 (s, 1H), 7.74 (s, 1H), 6.95 (s, 1H), 4.30 (s, 2H), 4.08˜4.05 (m, 1H), 3.89 (s, 1H), 3.80˜3.74 (m, 1H), 3.71˜3.67 (m, 3H), 3.22˜3.11 (m, 3H), 3.00˜2.95 (m, 2H), 2.63 (s, 3H), 2.52 (s, 1H), 2.41˜2.37 (m, 1H), 2.33˜2.30 (m, 1H), 2.15 (s, 1H), 2.04˜2.01 (m, 2H), 1.88˜1.75 (m, 3H), 1.17 (s, 1H), 1.16 (s, 3H).

LC-MS [mobile phase: from 50% water (0.1% TFA) and 50% CH₃CN (0.1% TFA) to 5% water (0.1% TFA) and 95% CH₃CN (0.1% TFA) in 2.6 min]: Rt=0.78 min; MS Calcd: 500.2, MS Found: 501.2 [M+H]⁺.

Chiral HPLC: Rt=4.413 min, ee: 99.7%

### Examples 28 and 29

### Cis-1-(3-fluoro-4-(1-(6-((R)-2-(hydroxymethyl)morpholino)-2-methylpyrimidin-4-yl)-5-methyl-1H-indazol-6-yl)piperidin-1-yl)propan-2-ol (Single Unknown Isomer 1, Rt=5.609 Min; Single Unknown Isomer 2, Rt=6.101 Min)

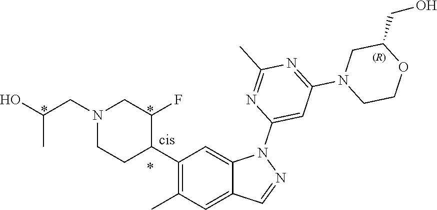

To a solution of 1-(cis-3-fluoro-4-(1-(6-((R)-2-(hydroxymethyl)morpholino)-2-methylp-yrimidin-4-yl)-5-methyl-1H-indazol-6-yl)piperidin-1-yl)propan-2-one (D90, 79 mg, 0.16 mmol, from peak 1) in MeOH (5 mL) was added NaBH₄ (12 mg, 0.32 mmol). The reaction mixture was stirred at rt for 1 h, quenched with water (0.5 mL) and diluted with DCM (30 mL). The separated organic layer was dried, filtered and concentrated to give the title product as a white solid (82 mg, crude)

LC-MS [mobile phase: from 95% water (0.1% TFA) and 5% CH₃CN (0.1% TFA) to 5% water (0.1% TFA) and 95% CH₃CN (0.1% TFA) in 2.0 min]: Rt=1.24 min; MS Calcd.: 498.3, MS Found: 499.4 [M+H]⁺.

### Chiral Separation:

Method: AD-H, Column size: 0.46 cm×15 cm, Mobile phase: CO₂/ETOH (0.1% NH₃H₂O)=60/40, Flow rate: 0.5 mL/min, Wave length: UV 254 nm, Temperature: 25° C.

### Peak 1 (E28): Single Unknown Isomer 1

¹H NMR (400 MHz, CDCl₃): δ 8.91 (s, 1H), 8.07 (s, 1H), 7.53 (s, 1H), 6.96 (s, 1H), 4.97˜4.79 (m, 1H), 4.31˜4.29 (m, 2H), 4.08˜4.05 (m, 1H), 3.92 (br, 1H), 3.81˜3.67 (m, 4H), 3.40˜3.34 (m, 2H), 3.14˜3.11 (m, 3H), 2.98˜2.91 (m, 1H), 2.61 (s, 3H), 2.62˜2.51 (m, 2H), 2.48 (s, 3H), 2.39˜2.33 (m, 1H), 2.15˜2.07 (m, 1H), 2.01˜1.97 (m, 2H), 1.90˜1.81 (m, 1H), 1.19 (d, J=6.0 Hz, 3H).

¹⁹F NMR (376 MHz, CDCl₃): δ 183.93 (s)

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=1.16 min; MS Calcd.: 498.3, MS Found: 499.4 [M+H]⁺.

Chiral HPLC: Rt=5.609 min, ee: 100%

### Peak 2 (E29): Single Unknown Isomer 2

¹H NMR (400 MHz, CDCl₃): δ 8.91 (s, 1H), 8.07 (s, 1H), 7.53 (s, 1H), 6.96 (s, 1H), 4.94˜4.76 (m, 1H), 4.33˜4.27 (m, 2H), 4.09˜4.05 (m, 1H), 3.93˜3.88 (m, 1H), 3.79˜3.67 (m, 4H), 3.52˜3.49 (m, 1H), 3.33 (brs, 1H), 3.15˜3.08 (m, 2H), 2.98˜2.91 (m, 2H), 2.62 (s, 3H), 2.50˜2.40 (m, 3H), 2.48 (s, 3H), 2.23˜2.20 (m, 1H), 2.01˜1.95 (m, 3H), 1.19 (d, J=6.0 Hz, 3H).

¹⁹F NMR (376 MHz, CDCl₃): δ 183.93 (s)

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=1.17 min; MS Calcd.: 498.3, MS Found: 499.4 [M+H]⁺.

Chiral HPLC: Rt=6.101 min, ee: 99.22%

### Examples 30 and 31

### 1-(cis-3-fluoro-4-(1-(6-((R)-2-(hydroxymethyl)morpholino)-2-methylpyrimidin-4-yl)-5-methyl-1H-indazol-6-yl)piperidin-1-yl)propan-2-ol (Single Unknown Isomer 1, Rt=4.967 Min; Single Unknown Isomer 2, Rt=5.706 Min)

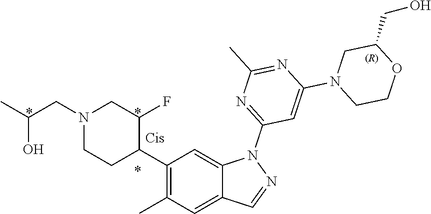

The title compounds were prepared by a procedure similar to that described for E28 and E29 starting from NaBH₄ and a solution of 1-(cis-3-fluoro-4-(1-(6-((R)-2-(hydroxy-methyl)morpholino)-2-methylpyrimidin-4-yl)-5-methyl-1H-indazol-6-yl)piperidin-1-yl)-propan-2-one (E95) in MeOH (5 mL) in one portion at rt for 1 hour.

LC-MS [mobile phase: from 95% water (0.1% TFA) and 5% CH₃CN (0.1% TFA) to 5% water (0.1% TFA) and 95% CH₃CN (0.1% TFA) in 2.0 min]: Rt=1.29 min; MS Calcd.: 498.28, MS Found: 499.4 [M+H]⁺.

### Chiral Separation:

Method: Column: AD-H; Column size: 0.46 cm×15 cm; Mobile phase: CO₂:EtOH (0.1% NH₃.H₂O)=60:40; Flow rate: 0.5 mL/min; Wave length: UV 254 nm; Temperature: 25° C.; Sample in EtOH

### Peak 1 (E30): Single Unknown Isomer 1

¹H NMR (400 MHz, CDCl₃) δ 8.91 (s, 1H), 8.07 (s, 1H), 7.53 (s, 1H), 6.96 (s, 1H), 4.93˜4.72 (m, 1H), 4.33˜4.27 (m, 2H), 4.09˜4.05 (m, 1H), 3.95˜3.90 (m, 1H), 3.80˜3.67 (m, 4H), 3.54˜3.48 (m, 1H), 3.33 (br, 1H), 3.15˜3.11 (m, 2H), 2.95˜2.91 (m, 2H), 2.62 (s, 3H), 2.47 (s, 3H), 2.51˜2.40 (m, 3H), 2.25˜2.18 (m, 1H), 1.98˜1.89 (m, 3H), 1.19 (d, J=6.0 Hz, 3H).

¹⁹F NMR (376 MHz, CDCl₃) δ 183.93 (s, 1F),

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=1.17 min; MS Calcd: 498.28, MS Found: 499.3 [M+H]⁺.

Chiral HPLC: Rt=4.967 min, ee 100%;

### Peak 2 (E31): Single Unknown Isomer 2

¹H NMR (400 MHz, CDCl₃) δ 8.92 (s, 1H), 8.07 (s, 1H), 7.53 (s, 1H), 6.96 (s, 1H), 4.98-4.86 (m, 1H), 4.33-4.28 (m, 2H), 4.08-4.05 (m, 1H), 3.96 (br, 1H), 3.78-3.67 (m, 4H), 3.39-3.36 (m, 2H), 3.15-3.11 (m, 3H), 2.97-2.91 (m, 1H), 2.62 (s, 3H), 2.47 (s, 3H), 2.60-2.37 (m, 3H), 2.16 (br, 1H), 2.02-1.97 (m, 3H), 1.19 (d, J=6.4 Hz, 3H).

¹⁹F NMR (376 MHz, CDCl₃) δ 184.01 (s, 1F)

LC-MS [mobile phase: from 90% water (0.1% TFA) and 10% CH₃CN (0.1% TFA) to 5% water (0.1% TFA) and 95% CH₃CN (0.1% TFA) in 2.0 min]: Rt=1.18 min; MS Calcd: 498.28, MS Found: 499.3 [M+H]⁺.

Chiral HPLC: Rt=5.706 min, ee 98.48%;

### Examples 32 and 33

### Cis-1-(3-fluoro-4-(1-(6-((S)-2-(hydroxymethyl)morpholino)-2-methylpyrimidin-4-yl)-5-methyl-1H-indazol-6-yl)piperidin-1-yl)propan-2-ol (Single Unknown Isomer 1, Rt=4.930 Min; Single Unknown Isomer 2, Rt=5.263 Min)

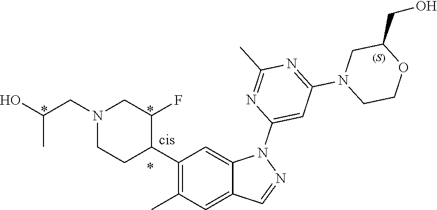

To a solution of 1-(cis-3-fluoro-4-(1-(6-((S)-2-(hydroxymethyl)morpholino)-2-methylpyr-imidin-4-yl)-5-methyl-1H-indazol-6-yl)piperidin-1-yl)propan-2-one (D91, 110 mg, 0.220 mmol, from peak 1) in MeOH (5 mL) was added NaBH₄ (42.0 mg, 1.10 mmol). The reaction mixture was stirred at rt for 1 h, diluted with sat. NH₄Cl (60 mL) and extracted with EtOAc (60 mL×3). The combined organic layers were dried and filtered. The filtrate was concentrated to give the title product (100 mg, crude) as a white solid.

LC-MS [mobile phase: from 95% water (0.1% TFA) and 5% CH₃CN (0.1% TFA) to 5% water (0.1% TFA) and 95% CH₃CN (0.1% TFA) in 10.0 min]: Rt=4.46 min; MS Calcd.: 498.3, MS Found: 499.4 [M+H]⁺.

### Chiral Separation:

Method: AD-H, Column size: 0.46 cm×15 cm, Mobile phase: CO₂:EtOH (0.1% NH₃.H₂O)=60:40, Flow rate: 0.5 mL/min, Wave length: UV 254 nm, Temperature: 25° C.

### Peak 1 (E32): Single Unknown Isomer 1

¹H NMR (400 MHz, CDCl₃): δ 8.91 (s, 1H), 8.07 (s, 1H), 7.53 (s, 1H), 6.96 (s, 1H), 4.94˜4.80 (m, 1H), 4.29˜4.27 (m, 2H), 4.06˜4.03 (m, 1H), 3.91 (br, 1H), 3.77˜3.75 (m, 1H), 3.71˜3.67 (m, 3H), 3.34˜3.32 (m, 2H), 3.14˜3.08 (m, 2H), 2.97˜2.95 (m, 1H), 2.84 (s, 1H), 2.61 (s, 3H), 2.53˜2.50 (m, 1H), 2.48 (s, 3H), 2.39˜2.33 (m, 2H), 2.11˜2.09 (m, 1H), 2.00˜1.98 (m, 2H), 1.97˜1.85 (m, 1H), 1.19˜1.18 (d, J=6.0 Hz, 3H).

¹⁹F NMR (376 MHz, CDCl₃): δ 183.391 (s)

LC-MS [mobile phase: from 95% water (0.1% TFA) and 5% CH₃CN (0.1% TFA) to 5% water (0.1% TFA) and 95% CH₃CN (0.1% TFA) in 9.0 min]: Rt=4.33 min; MS Calcd.: 498.3, MS Found: 499.3 [M+H]⁺.

Chiral HPLC: Rt=4.930 min, ee: 100%

### Peak 2 (E33): Single Unknown Isomer 2

¹H NMR (400 MHz, CDCl₃): δ 8.90 (s, 1H), 8.07 (s, 1H), 7.53 (s, 1H), 6.95 (s, 1H), 4.92˜4.79 (m, 1H), 4.30˜4.27 (m, 2H), 4.06˜4.05 (m, 1H), 3.92˜3.90 (m, 1H), 3.77˜3.72 (m, 1H), 3.69˜3.65 (m, 3H), 3.51˜3.50 (m, 1H), 3.33˜3.31 (m, 1H), 3.15˜3.08 (m, 2H), 2.94˜2.91 (m, 2H), 2.61 (s, 3H), 2.56 (s, 3H), 2.47˜2.40 (m, 2H), 2.22˜2.18 (s, 1H), 1.96˜1.94 (m, 3H), 1.61˜1.58 (m, 1H), 1.20˜1.18 (d, J=6.4 Hz, 3H).

¹⁹F NMR (376 MHz, CDCl₃): δ 183.917 (s)

LC-MS [mobile phase: from 95% water (0.1% TFA) and 5% CH₃CN (0.1% TFA) to 5% water (0.1% TFA) and 95% CH₃CN (0.1% TFA) in 9.0 min]: Rt=4.34 min; MS Calcd.: 498.3, MS Found: 499.4 [M+H]⁺.

Chiral HPLC: Rt=5.263 min, ee: 100%

### Examples 34 and 35

### Cis-1-(3-fluoro-4-(1-(6-((S)-2-(hydroxymethyl)morpholino)-2-methylpyrimidin-4-yl)-5-methyl-1H-indazol-6-yl)piperidin-1-yl)propan-2-ol (from Peak 2) (Single Unknown Isomer 1, Rt=4.861 Min; Single Unknown Isomer 2, Rt=5.947 Min)

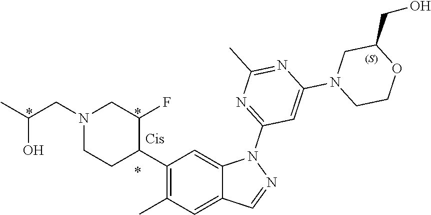

The title compounds were prepared by a procedure similar to that described for E27 and E28 starting from NaBH₄ and a solution of 1-(cis-3-fluoro-4-(1-(6-((S)-2-(hydroxymeth-yl)morpholino)-2-methylpyrimidin-4-yl)-5-methyl-1H-indazol-6-yl)piperidin-1-yl)propan-2-one (D96).

LC-MS [mobile phase: from 95% water (0.1% TFA) and 5% CH₃CN (0.1% TFA) to 5% water (0.1% TFA) and 95% CH₃CN (0.1% TFA) in 2.0 min]: Rt=1.30 min; MS Calcd.: 498.3, MS Found: 499.4 [M+H]⁺.

### Chiral Separation:

Method: AD-H, Column size: 0.46 cm×15 cm, Mobile phase: CO₂:EtOH (0.1% NH₃.H₂O)=60:40, Flow rate: 0.5 mL/min, Wave length: UV 254 nm, Temperature: 25° C.

### Peak 1 (E34): Single Unknown Isomer 1

¹H NMR (400 MHz, CDCl₃): δ 8.90 (s, 1H), 8.07 (s, 1H), 7.53 (s, 1H), 6.95 (s, 1H), 4.90˜4.77 (m, 1H), 4.32˜4.29 (m, 2H), 4.07˜4.05 (m, 1H), 3.92 (br, 1H), 3.79˜3.74 (m, 1H), 3.71˜3.67 (m, 3H), 3.52˜3.49 (m, 1H), 3.32 (br, 1H), 3.13˜3.11 (m, 2H), 2.94˜2.91 (m, 2H), 2.61 (s, 3H), 2.50 (s, 3H), 2.48˜2.40 (m, 2H), 2.18˜2.17 (m, 1H), 2.04 (br, 1H), 1.97˜1.94 (m, 2H), 1.19˜1.18 (d, J=6.0 Hz, 3H).

¹⁹F NMR (376 MHz, CDCl₃): δ 183.913 (s)

LC-MS [mobile phase: from 95% water (0.1% TFA) and 5% CH₃CN (0.1% TFA) to 5% water (0.1% TFA) and 95% CH₃CN (0.1% TFA) in 9.0 min]: Rt=4.33 min, MS Calcd.: 498.3, MS Found: 499.3 [M+H]⁺.

Chiral HPLC: Rt=4.861 min, ee: 100%

### Peak 2 (E35): Single Unknown Isomer 2

¹H NMR (400 MHz, CDCl₃): δ 8.91 (s, 1H), 8.07 (s, 1H), 7.53 (s, 1H), 6.95 (s, 1H), 4.97˜4.84 (m, 1H), 4.30˜4.28 (m, 2H), 4.09˜4.04 (m, 1H), 3.93 (br, 1H), 3.74˜3.70 (m, 1H), 3.69˜3.67 (m, 3H), 3.39˜3.34 (m, 1H), 3.14˜3.11 (m, 3H), 2.94˜2.90 (m, 1H), 2.84 (br, 1H), 2.62 (s, 3H), 2.54˜2.50 (m, 2H), 2.41 (s, 3H), 2.37˜2.34 (m, 1H), 2.16 (s, 1H), 2.16˜2.01 (m, 2H), 1.63˜1.62 (m, 1H), 1.20˜1.18 (d, J=6.4 Hz, 3H).

¹⁹F NMR (376 MHz, CDCl₃): δ 183.593 (s)

LC-MS [mobile phase: from 95% water (0.1% TFA) and 5% CH₃CN (0.1% TFA) to 5% water (0.1% TFA) and 95% CH₃CN (0.1% TFA) in 9.0 min]: Rt=4.34 min, MS Calcd.: 498.3, MS Found: 499.4 [M+H]⁺.

Chiral HPLC: Rt=5.947 min, ee: 100%

### Example 36

### 1-(2-methyl-6-(5-methyl-6-(1-(3,3,3-trifluoro-2-hydroxypropyl)piperidin-4-yl)-1H-indazol-1-yl)pyrimidin-4-yl)azetidin-3-ol

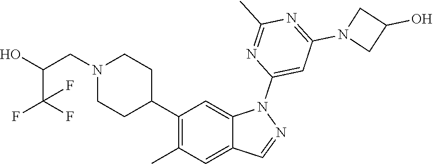 [SMILES: Cc1nc(N2CC(O)C2)cc(-n2ncc3cc(C)c(C4CCN(CC(O)C(F)(F)F)CC4)cc32)n1]

To a solution of 1-(2-methyl-6-(5-methyl-6-(piperidin-4-yl)-1H-indazol-1-yl)pyrimidin-4-yl)azetidin-3-ol (D98, 330 mg, 0.870 mmol) in acetonitrile (4 mL) was added 2-(trifluorom-ethyl)oxirane (489 mg, 4.40 mmol). The reaction mixture was stirred at 30° C. overnight and concentrated. The crude was purified by flash chromatography (DCM/MeOH=20:1) to give the title product (200 mg, 47%) as a white solid.

¹HNMR (400 MHz, DMSO-d₆): δ 8.74 (s, 1H), 8.30 (s, 1H), 7.61 (s, 1H), 6.54 (s, 1H), 6.13 (d, J=5.2 Hz, 1H), 5.79 (d, J=6.8 Hz, 1H), 4.63-4.61 (m, 1H), 4.31-4.27 (m, 2H), 4.19 (s, 1H), 3.83-3.79 (m, 2H), 3.31 (s, 9H), 3.10-3.07 (m, 2H), 2.33-2.21 (m, 2H), 1.83-1.67 (m, 4H).

LCMS [column: Waters X-bridge C18 5 um; column size: 4.6 mm×50 mm; mobile phase: B (CH₃CN), A (0.02% NH₄Ac in water); gradient (B %) in 6 mins]: Rt=2.966 min, MS Calcd.: 490, MS Found: 491 [M+H]⁺.

### Example 37

### 1-fluoro-3-(4-(1-(6-((S)-2-(hydroxymethyl)morpholino)-2-methylpyrimidin-4-yl)-5-methyl-1H-indazol-6-yl)piperidin-1-yl)propan-2-ol

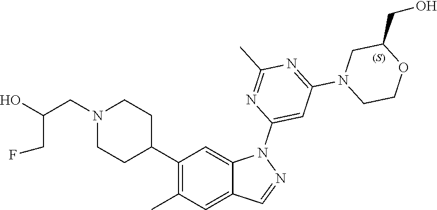

To a suspension of 1-fluoro-3-(4-(5-methyl-1H-indazol-6-yl)piperidin-1-yl)propan-2-ol (D100, 150 mg, 0.510 mmol), (S)-(4-(6-iodo-2-methylpyrimidin-4-yl)morpholin-2-yl)methanol (D113, 173 mg, 0.510 mmol), CuI (98.0 mg, 0.510 mmol) and K₃PO₄ (219 mg, 1.03 mmol) in toluene (10 mL) was added N₁,N₂-dimethylethane-1,2-diamine (91.0 mg, 1.03 mmol). The resulting mixture was degassed with N₂ three times, stirred at 80° C. for 2 h under N₂, diluted with EtOAc (30 mL), washed with sat. NH₄Cl (30 mL×2) and brine (30 mL), dried over anhydrous Na₂SO₄ and concentrated. The residue was purified by Prep-TLC (CH₂Cl₂: MeOH=10:1) followed by C₁₈ eluted with MeCN/H₂O (from 5/95 to 95/5) to give the title product (60 mg, yield: 23%) as a white solid.

¹H NMR (400 MHz, CDCl₃): δ 8.80 (s, 1H), 8.06 (s, 1H), 7.50 (s, 1H), 6.96 (s, 1H), 4.60˜4.57 (m, 0.5H), 4.49˜4.45 (m, 1H), 4.37˜4.27 (m, 2.5H), 4.08˜3.95 (m, 2H), 3.77˜3.65 (m, 5H), 3.20˜2.82 (m, 5H), 2.64 (s, 3H), 2.59˜2.49 (m, 3H), 2.46 (s, 3H), 2.23˜2.17 (m, 1H), 2.00˜1.81 (m, 5H).

¹⁹F NMR (376 MHz, CDCl₃): δ −231.972

LC-MS [mobile phase: from 90% water (0.1% TFA) and 10% CH₃CN (0.1% TFA) to 5% water (0.1% TFA) and 95% CH₃CN (0.1% TFA) in 9 min]: Rt=4.41 min; MS Calcd: 498.3, MS Found: 499.3 [M+H]⁺.

### Example 38

### 2-fluoro-3-(4-(1-(6-((S)-2-(hydroxymethyl)morpholino)-2-methylpyrimidin-4-yl)-5-methyl-1H-indazol-6-yl)piperidin-1-yl)propan-1-ol

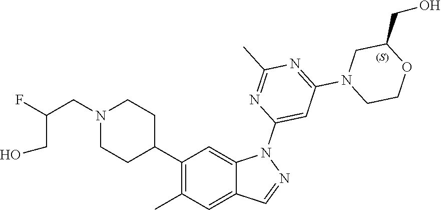

To a suspension of 2-fluoro-3-(4-(5-methyl-1H-indazol-6-yl)piperidin-1-yl)propan-1-ol (D100, 150 mg, 0.510 mmol), (S)-(4-(6-iodo-2-methylpyrimidin-4-yl)morpholin-2-yl)methanol (D113, 173 mg, 0.510 mmol), CuI (98.0 mg, 0.510 mmol) and K₃PO₄ (219 mg, 1.03 mmol) in toluene (10 mL) was added N₁,N₂-dimethylethane-1,2-diamine (91.0 mg, 1.03 mmol). The resulting mixture was degassed with N₂ three times, stirred at 80° C. for 3 h under N₂, diluted with DCM (50 mL), washed with sat. NH₄Cl (50 mL) and brine (50 mL). The organic solution was dried over anhydrous Na₂SO₄ and concentrated. The residue was purified by silica gel chromatography (CH₂Cl₂:MeOH=10:1) followed by C18 eluted with MeCN/H₂O (from 5/95 to 95/5) to give the title product (85.0 mg, yield: 33.0%) as a white solid.

¹H NMR (400 MHz, CDCl₃): δ 8.77 (s, 1H), 8.05 (s, 1H), 7.50 (s, 1H), 6.95 (s, 1H), 4.83˜4.78 (m, 0.5H), 4.71˜4.66 (m, 0.5H), 4.32˜4.28 (m, 2H), 4.08˜4.04 (m, 1H), 3.97˜3.95 (m, 1H), 3.92˜3.90 (m, 1H), 3.79˜3.65 (m, 4H), 3.32˜3.29 (m, 1H), 3.18˜3.07 (m, 2H), 2.98˜2.82 (m, 4H), 2.64 (s, 3H), 2.45 (s, 3H), 2.38˜2.29 (m, 2H), 1.94˜1.88 (m, 4).

¹⁹F NMR (376 MHz, CDCl₃): δ −190.88

LC-MS [mobile phase: from 80% water (0.1% TFA) and 20% CH₃CN (0.1% TFA) to 5% water (0.1% TFA) and 95% CH₃CN (0.1% TFA) in 9 min]: Rt=3.50 min; MS Calcd: 498.3, MS Found: 499.3 [M+H]⁺.

### Example 39

### (S)-(4-(2-methyl-6-(5-methyl-6-(1-(2-(methylsulfonyl)ethyl)piperidin-4-yl)-1H-indazol-1-yl)pyrimidin-4-yl)morpholin-2-yl)methanol

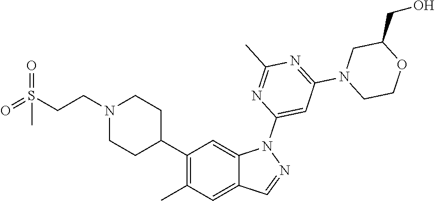 [SMILES: Cc1nc(N2CCO[C@H](CO)C2)cc(-n2ncc3cc(C)c(C4CCN(CCS(C)(=O)=O)CC4)cc32)n1]

To a mixture of (S)-(4-(2-methyl-6-(5-methyl-6-(piperidin-4-yl)-1H-indazol-1-yl)pyrimi-din-4-yl)morpholin-2-yl)methanol (D100, 120 mg, 0.284 mmol) and Na₂CO₃ (75.0 mg, 0.710 mmol) in THF (5 mL) was added (methylsulfonyl)ethene (66.0 mg, 0.625 mmol). The reaction mixture was stirred at 65° C. for 3 hours, diluted with H₂O (20 mL) and extracted with EtOAc (20 mL×2). The combined organic layers were concentrated and purified by preparative TLC (DCM/MeOH=20/1) to give the title product (19.0 mg, 13.0%) as a yellow solid.

¹HNMR (400 MHz, CDCl₃): δ 8.79 (s, 1H), 8.06 (s, 1H), 7.50 (s, 1H), 6.95 (s, 1H), 4.30 (t, J=11.2 Hz, 2H), 4.09˜4.05 (m, 1H), 3.81˜3.77 (m, 1H), 3.75˜3.65 (m, 3H), 3.22 (br s, 2H), 3.15˜3.08 (m, 6H), 2.98˜2.92 (m, 3H), 2.86 (t, J=11.2 Hz, 1H), 2.60 (s, 3H), 2.46 (s, 3H), 2.28 (t, J=10.4 Hz, 2H), 1.98˜1.82 (m, 5H).

LC-MS [column: C18; column size: 4.6×50 mm; mobile phase: B (MeCN), A (0.02% NH₄Ac in water); gradient (B %)]: Rt=3.637 min, MS Calcd.: 528, MS Found: 529 [M+H]⁺.

### Example 40

### 1-(6-(azetidin-1-yl)-2-methoxypyrimidin-4-yl)-5-methyl-6-(1-(2-(methylsulfonyl)eth-yl)piperidin-4-yl)-1H-indazole

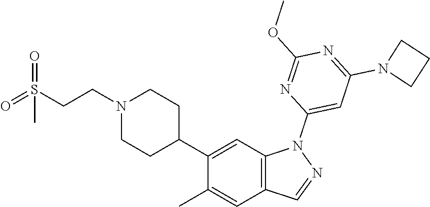 [SMILES: COc1nc(N2CCC2)cc(-n2ncc3cc(C)c(C4CCN(CCS(C)(=O)=O)CC4)cc32)n1]

To a mixture of 1-(6-(azetidin-1-yl)-2-methoxypyrimidin-4-yl)-5-methyl-6-(Piperidin-4-yl)-1H-indazole (D112,150 mg, 0.40 mmol) and Na₂CO₃ (110 mg, 1.00 mmol) in THF (5 mL) was added (methylsulfonyl)ethene (93.0 mg, 0.880 mmol). The reaction mixture was stirred at 65° C. for 2 hours, diluted with H₂O (20 mL) and extracted with EtOAc (20 mL×2). The combined organic layers were concentrated, triturated with MeOH (5 mL) and filtered. The cake was purified by preparative TLC (DCM/MeOH=20/1) to give the title product (27.0 mg, 14.0%) as a white solid.

¹HNMR (400 MHz, CDCl₃): δ 8.74 (s, 1H), 8.06 (s, 1H), 7.51 (s, 1H), 6.43 (s, 1H), 4.17 (t, J=7.2 Hz, 4H), 4.09 (s, 3H), 3.20 (t, J=6.0 Hz, 2H), 3.10-3.09 (m, 5H), 2.98-2.93 (m, 2H), 2.85 (t, J=11.2 Hz, 1H), 2.45-2.40 (m, 5H), 2.25 (t, J=11.2 Hz, 2H), 1.94-1.91 (m, 2H), 1.83-1.75 (m, 2H).

LC-MS [column: C₁₈; column size: 4.6×50 mm; mobile phase: B (CH₃CN), A (0.02% NH₄Ac in water); gradient (B %)]: Rt=3.817 min, MS Calcd.: 484, MS Found: 485 [M+H]⁺.

### Example 41

### (R)-(4-(2-methyl-6-(5-methyl-6-(1-(2-(methylsulfonyl)ethyl)piperidin-4-yl)-1H-indaz-ol-1-yl)pyrimidin-4-yl)morpholin-2-yl)methanol

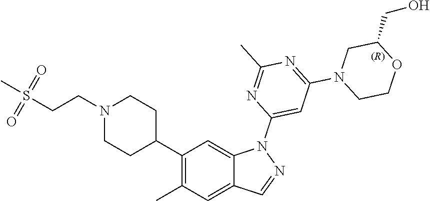

The title compound was prepared by a procedure similar to that described for E40 starting from a mixture of (R)-(4-(2-methyl-6-(5-methyl-6-(piperidin-4-yl)-1H-indazol-1-yl)pyri-midin-4-yl)morpholin-2-yl)methanol (D109), Na₂CO₃, (methylsulfonyl)ethene in THF at 65° C. for 3 hrs.

¹HNMR (400 MHz, CDCl₃): δ 8.79 (s, 1H), 8.06 (s, 1H), 7.50 (s, 1H), 6.59 (s, 1H), 4.33-4.26 (m, 2H), 4.09-4.05 (m, 1H), 3.80-3.65 (m, 4H), 3.23-3.19 (m, 2H), 3.14-3.11 (m, 6H), 2.98-2.95 (m, 3H), 2.89-2.83 (m, 1H), 2.60 (s, 3H), 2.46 (s, 3H), 2.30-2.25 (m, 2H), 1.99-1.84 (m, 5H).

LCMS [column: Waters X-bridge C₁₈ 5 μm; column size: 4.6 mm×50 mm; mobile phase: B (CH₃CN), A (0.02% NH₄Ac in water); gradient (B %) in 6 mins]: Rt=3.375 min, MS Calcd.: 528, MS Found: 529 [M+H]⁺.

### Example 42

### 1-(2-methoxy-6-(5-methyl-6-(1-(2-(methylsulfonyl)ethyl)piperidin-4-yl)-1H-indazol-1-yl)pyrimidin-4-yl)azetidin-3-ol

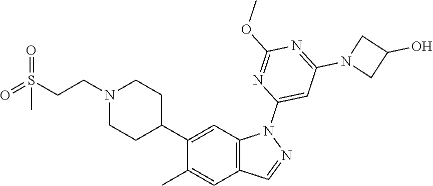 [SMILES: COc1nc(N2CC(O)C2)cc(-n2ncc3cc(C)c(C4CCN(CCS(C)(=O)=O)CC4)cc32)n1]

The title compound was prepared by a procedure similar to that described for E40 starting from a solution of 1-(2-methoxy-6-(5-methyl-6-(piperidin-4-yl)-1H-indazol-1-yl)pyrimi-din-4-yl)azetidin-3-ol and Na₂CO₃ in THF.

¹HNMR (400 MHz, CDCl₃): δ 8.73 (s, 1H), 8.07 (s, 1H), 7.51 (s, 1H), 6.47 (s, 1H), 4.83 (br s, 1H), 4.43-4.39 (m, 2H), 4.09 (s, 3H), 4.03-3.99 (m, 2H), 3.21-3.17 (m, 2H), 3.10-3.07 (m, 5H), 2.95-2.84 (m, 3H), 2.45 (s, 3H), 2.29-2.21 (m, 3H), 1.94-1.76 (m, 4H).

LC-MS [column: C₁₈; column size: 4.6 mm×50 mm; mobile phase: B (CH₃CN), A (0.02% NH₄Ac in water); gradient (B %)]: Rt=3.531 min, MS Calcd.: 500, MS Found: 501 [M+H]⁺.

### Example 43

### 1-(2-methyl-6-(5-methyl-6-(1-(2-(methylsulfonyl)ethyl)piperidin-4-yl)-1H-indazol-1-yl)pyrimidin-4-yl)azetidin-3-ol

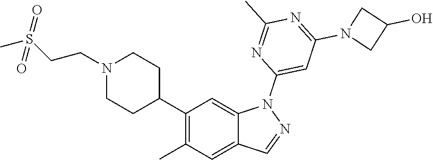 [SMILES: Cc1nc(N2CC(O)C2)cc(-n2ncc3cc(C)c(C4CCN(CCS(C)(=O)=O)CC4)cc32)n1]

To a mixture 1-(2-methyl-6-(5-methyl-6-(piperidin-4-yl)-1H-indazol-1-yl) pyrimidin-4-yl)azetidin-3-ol (D98,100 mg, 0.260 mmol) was in EtOH (5 mL) was added 1-bromo-2-(methylsulfonyl)ethane (35.0 mg, 0.310 mmol). The reaction mixture was stirred at rt for 2 hrs and concentrated. The residue was purified by prep-HPLC eluted with CH₃CN/H₂O (0.1% TFA, from 15/85 to 95/5) to give the title product as a white solid (18.0 mg, yield: 14.0%).

¹H NMR (400 MHz, CDCl₃): δ 8.73 (s, 1H), 8.01 (s, 1H), 7.46 (s, 1H), 6.53 (s, 1H), 5.58 (br, 1H), 4.75 (br, 1H), 4.39-4.34 (m, 2H), 3.96-3.95 (m, 2H), 3.57-3.51 (m, 3H), 3.35-3.34 (m, 2H), 3.01 (s, 3H), 2.71 (br, 1H), 2.56 (s, 3H), 2.40-2.37 (m, 4H), 2.16-2.13 (m, 2H), 2.04-2.00 (m, 2H), 1.99-1.97 (m, 2H).

LC-MS [mobile phase: from 80% water (0.1% TFA) and 20% CH₃CN (0.1% TFA) to 5% water (0.1% TFA) and 95% CH₃CN (0.1% TFA) in 9.0 min]: Rt=2.56 min, MS Calcd.: 484.6, MS Found: 485.3 [M+H]⁺.

### F. Assays and Data

As stated above, the compounds of present invention are LRRK2 kinase inhibitors, and may be useful in the treatment of diseases mediated by LRRK2. The biological activities and/or properties of the compounds of present invention can be determined using any suitable assay, including assays for determining the activity of a candidate compound as a LRRK2 kinase inhibitor, as well as tissue and in vivo models.

### 1. Assays

a. Full Length G2019 Human LRRK2 Inhibition Mass Spectrometry Assay

This assay for Leucine Rich Repeat Kinase 2 (LRRK2) inhibition is based on the direct measurement of the peptide ‘LRRKtide’ (LRRKtide: RLGRDKYKT*LRQIRQ and “*” refers to the site of phosphorylation) and phosphorylated ‘LRRKtide’ using a high throughput RapidFire mass spectrometry assay. Inhibitors are compounds that reduce the conversion of LRRKtide to phospho-LRRKtide.

### Human G2019 LRRK2 Plasmid Preparation

Primers used for PCR cloning:
pHTBV-F:SEQ ID No: 1
LRRK2 wt-F1:SEQ ID No: 2
LRRK2 wt-R1: SEQ ID No: 3
LRRK2 wt-F2: SEQ ID No: 4
LRRK2 wt-R2: SEQ ID No: 5
LRRK2 wt-F3:SEQ ID No: 6
pHTBV-R: SEQ ID No: 7

pHTBV1-N-Flag-hu LRRK2 was generated by PCR amplifying the full length LRRK2 sequence with N terminal Flag tag from pcDNA3.1(+)_Human_LRRK2 (NCBI Reference Sequence: NP_940980.3) with the primers described above, and cloned into pHTBV1mcs3 vector between BamHI and KpnI sites.

The G2019 full length Flag-LRRK2 coding sequence is SEQ ID No: 8.

The translated amino acid sequence for human G2019 full length N terminal flag tagged LRRK2 protein is SEQ ID No: 9.

### Insect Cell Cultures

Sf9 insect cells (Invitrogen Life Technologies, Carlsbad, Calif.) were maintained at 27° C. in SF 900 11 SFM in 500-ml shaker flasks (Erlenmeyer, Corning). The cells were maintained in exponential growth phase and subcultured twice per week. For larger volumes, cells were grown in 2-liter shaker flasks (Erlenmeyer, Corning) while being agitated with 120 rpm at 27° C. incubator shaker.

### Generation of the BacMam Virus

To generate the recombinant BacMam virus, DH10Bac competent cells (10361-012, Invitrogen) were transformed by the genotypically normal human LRRK2 BacMam plasmid to generate the recombinant baculovirus DNA. The Sf9 insect cells were co-transfected with the mixture of recombinant baculovirus DNA and cellfectin (10362-100, Invitrogen). After 4 h of incubation at 27° C., the transfection media was replaced with Sf-900 III SFM medium containing 5% HI FBS (Ser. No. 10/100,147, Invitrogen). The cells were further incubated for 4 days. The infected cell culture medium containing the baculovirus (P0 virus stock) was collected and amplified by further infecting the 200 ml Sf9 cells via 200-300 ul P0.

### Quantification of BacMam Viral Titre by BacPAKRapid Titer

The viral titre, measured as plaque forming unit (pfu)/ml was determined using BacPAK Papid Titer Kit (631406, Clontech) according to the manufacturer's protocol. The Sf9 cells seeded in 96-well plate with 3×10⁵ cells per well were incubated with serial dilution of the viral stocks for 1 h at 27° C., 50 μl methyl cellulose overlay was added to each well followed by 43-47 h incubation. The cells were then fixed in 4% paraformaldehyde (PFA). After blocking the cells with diluted normal goat serum, Mouse anti-gp64 antibody was added to the cells. After 30 min incubation, the cells were washed with phosphate buffered saline containing 0.2% Triton-X100 (PBST) and incubated for another 30 min with goat anti-mouse antibody/HRP conjugate. This was followed by blue peroxidase substrate which detects the single infected cells and foci of infected cells by their dark blue color.

### Protein Expression & Purification

a) Expression of Flag Tagged Full Length G2019 Human LRRK2

HEK293 6E cells were incubated in a 37° C. incubator with a humidified atmosphere of 5% CO₂ on an orbital shaker rotating at 110 rpm. On the day of transduction, the cell viability was higher than 98% and the cell density was in the range of 1×10⁶-1.5×10⁶ cells/ml. HEK293 6E cells were centrifuged at 1,000 rpm for 10 min, and then the cells were resuspended in fresh Freestyle 293 expression medium (Invitrogen:12338) with 0.1% F-68(Invitrogen:24040-032) but without antibiotics (G418) at density of 1×10⁶ cells/ml. BacMam virus with Flag-hu LRRK2 (genotypically normal) gene was centrifuged at 40,000 g for 2 hours, then resuspended in fresh Freestyle 293 expression medium. The resuspended virus was added into the cells in at MOI of 10. The cells were incubated in a 37° C. incubator with a humidified atmosphere of 5% CO₂ in air on an orbital shaker rotating at 110 rpm. Cultures were harvested at approximately 48 hours post-transduction by centrifugation at 4,000 rpm for 20 min and pellets were frozen for purification.

b) Purification of Flag Tagged Full Length G2019 Human LRRK2

The cell pellet was resuspended in (20 mL/liter cell culture) lysis buffer (50 mM TrisHCl pH7.5 at 4° C., 500 mM NaCl, 0.5 mM EDTA, 0.1% TritonX-100, 10% glycerol, freshly add 2 mM DTT), with protease inhibitors (Roche: 04693132001) and benzonase (Merck Millipore: 70746-3CN) at recommended concentration suggested by suppliers. The suspended cells were lysed by sonication on ice for 30 min (2 secs on/4 sec off, 20% amplitude), and centrifuged at 10,000 rpm for 30 minutes at 4° C. The supernatant was incubated with 1 mL per litre of cell culture of anti-Flag magnetic beads (Sigma-Aldrich: M8823) at 4° C. for 3 hours, then the beads were washed by 5 mL (5 column volume) binding buffer (50 mM Tris pH7.5@ 4 C, 500 mM NaCl, 0.5 mM EDTA, 0.1% TritonX-100, 10% glycerol, freshly add 2 mM DTT) for three times. The Flag tagged LRRK2 proteins were eluted by Elution buffer (50 mM Tris pH7.5@ 4 C, 500 mM NaCl, 0.5 mM EDTA, 0.1% TritonX-100, 10% glycerol, freshly add 2 mM DTT, 250 ug/ml Flag peptide (Sigma-Aldrich:F3290)) at 4° C. for 2 hours. Flag peptide was removed by Zeba Spin Desalting Columns, 7K MWCO (Thermo-Fisher: 89893) and the buffer of eluted LRRK2 proteins was exchanged into Storage Buffer (50 mM Tris pH7.5@4 C, 150 mM NaCl, 0.5 mM EDTA, 0.02% Triton X-100, 2 mM DTT and 50% Glycerol) using Amicon Ultra Centrifugal Filter Units (100 kD) (Merck: UFC910096). Fractions containing LRRK2 proteins were pooled, aliquoted and stored at −80° C. Protein concentration was determined by Bradford protein assay, and protein purity was analyzed by NuPAG Novex 4-12% Bis-Tris Protein Gels (Invitrogen: NP0322BOX).

### Assay Protocol

1) A 10 mM test compound was dissolved in 100% DMSO and serially diluted 1 in 4. 100 nL of this dilution series was then added to a 384 well, v bottom polypropylene plate, excluding columns 6 and 18. 100 nL of DMSO was added to columns 6 and 18 as controls wells. Assay dilution gave a top final assay concentration of test compound of 100 μM
2) 50 μl of 1% formic acid in laboratory grade water was added to column 18 using a multidrop combi dispenser to act as a pre stopped assay control.
3) 5 μl of ‘enzyme solution’ containing 50 nM of purified recombinant Full length Flag-LRRK2 in assay buffer (50 mM Hepes (pH 7.2), 10 mM MgCl2, 150 mM NaCl, 5% glycerol, 0.0025% triton X-100 and 1 mM DTT) was added to all wells using a multidrop combi dispenser, giving a final assay concentration of 25 nM LRRK2 enzyme. This resulted in column 6 (enzyme plus DMSO) giving 0% inhibition and column 18 giving 100% inhibition (pre stopped control). Test plates were then incubated for 30 minutes at room temperature.
4) 5 μl ‘substate solution’ containing 50 uM LRRKtide peptide substrate and 4 mM ATP was added to all wells of the plate using a multidrop combi dispenser giving a final assay concentration of 25 uM LRRKtide and 2 mM ATP. Test plates were then incubated for 1 hour at room temperature. (Incubation may vary depending on rate and linearity of reaction with different enzyme batches).
5) 50 μl of 1% formic acid in laboratory grade water was added to all wells (minus column 18) to quench the reaction, and plates were centrifuged at 3000 rpm for 10 minutes. Test plates were then analysed on an Agilent RapidFire High Throughput solid phase extraction system coupled to AB Sciex API 4000 triple quadropole mass spectrometer with the following setting:

RapidFire settings:
Sip Height=2 mm, Aspirate=500 ms, Load time=3000 ms, Elution time=3000 ms, Requilibration=500 ms.
Flow rates: pump 1=1.5 mL/min, pump 2 1.25 mL/min pump 3=0.8 mL/min Mass Spectrometer Settings:
LRRKtide Detection settings: Q1 mass 644.8 Da, Q3 mass 638.8, declustering potential 76 volts, collision energy 37 volts, CXP 34 volts.
Phospho-LRRKtide Detection settings: Q1 mass 671.4 Da, Q3 mass 638.8, Declustering potential 76 volts, Collision energy 37 volts, CXP 34 volts.
A C4 cartridge was used and running buffers were: A (aqueous) 0.1% formic acid in water B (organic) 0.1% formic acid, 80% acetonitrile, 20% water.
Collision gas: 12, Curtain gas: 25, Ion Source gas (1): 60, Ion Source gas (2): 60, Ion Spray Voltage: 5500, Temperature: 600, Interfaec Heater: ON.
Resolution Q1: low, Resolution Q3: low.
6) Data was analysed using ActivityBase software (IDBS). A percent conversion from LRRKtide to Phospho-LRRKtide was calculated using the following formula:

% conversion=(Phospho-LRRKtide product peak area/(Phospho-LRRKtide product peak area+LRRKtide substrate peak area))*100

b. Recombinant Cellular LRRK2 AlphaScreen Assay

To determine the activity of compounds against LRRK2 kinase activity in cells, the observed LRRK2 kinase-dependent modulation of LRRK2 Ser 935 phosphorylation (Dzamko et al., 2010, Biochem. J. 430: 405-413) was utilized to develop a quantitative 384 well plate-based immunoassay of LRRK2 Ser935 phosphorylation in the human neuroblastoma cell line SH-SY5Y, engineered to over-express recombinant full length LRRK2 protein.

A BacMam virus expressing full length recombinant LRRK2 was purchased from Invitrogen and amplified by inoculation of SF-9 cells at MOI 0.3 for 4-5 days in Sf-900 III SFM medium supplemented with 3% fetal bovine serum. Infected cell cultures were then centrifuged at 2000 g for 20 minutes, viral supernatant titer determined by anti-gp64 plaque assay and stored at 4° C.

Affinity-purified anti-phospho LRRK2 Ser935 sheep polyclonal antibody (Dzamko et al., 2010, Biochem. J. 430: 405-413) was biotinylated by standard methods (PerkinElmer). Anti-LRRK2 rabbit polyclonal antibody was purchased from Novus Biologicals. AlphaScreen Protein A IgG Kit (including acceptor and donor beads) was purchased from Perkin Elmer.

SH-SY5Y cells were grown in DMEM/F12 medium with 10% dialysed fetal bovine serum and harvested by treatment with 0.5% trypsin-EDTA for 5 minutes at 37° C. followed by centrifugation at 1000 rpm for 4 minutes. The cell pellet was resuspended in Opti-MEM reduced serum media (Invitrogen) at 200,000 cells/ml and mixed with the BacMam LRRK2 virus at M01=50. 50 μl cell solutions were then dispensed to each well of a 384-well plate and incubated at 37° C., 5% CO₂ for 24 hours.

Serial dilutions of test compounds were prepared in Opti-MEM reduced serum media (Invitrogen) and 5.6 ul transferred from compound plate to cell assay plate to achieve a top final assay concentration of 10 uM. DMSO was used in certain wells as controls. Cells were incubated at 37° C., 5% CO₂ for 60 minutes. The medium was then removed and cells lysed by addition of 20 ul cell lysis buffer (Cell Signaling Technology) and incubation at 4° C. for 20 minutes. 10 ul of antibody/acceptor bead mix [(1/1000 biotinylated-pS935 LRRK2 antibody, 1/1000 total-LRRK2 antibody, 1/100 Acceptor beads in AlphaScreen detection buffer (25 mM Hepes (pH 7.4), 0.5% Triton X-100, 1 mg/ml Dextran 500 and 0.1% BSA)] was then added to each well and plates incubated for 2 hours at ambient temperature in the dark. 10 μl of donor beads solution (1/33.3 donor beads in AlphaScreen detection buffer) was then added to each well. Following incubation for a further 2 hours at ambient temperature in the dark, plates were read on an EnVision™ plate reader at emission 520-620 nm with excitation 680 nm. Dose response curve data was based on sigmoidal dose-response model.

c. FASSIF Solubility Assay

Compound solubility may be evaluated in the fasted state simulated intestinal media (FaSSIF) at pH 6.5. Certain amount of test compound was admixed with certain volume of FaSSIF to prepare a suspension of about 1 mg/ml. The suspension was incubated at 37° C. in the water bath shaker for 24 hours. At the 4^{th} and 24^{th} hour, the suspension was centrifugated at 14K rpm for 15 minutes. 100 μl of the supernatant was withdrawn and diluted with the same volume of 50% acetonitrile water solution and analysed with UPLC (Ultra performance Liquid Chromatography). FaSSIF solubility was calculated based on the peak area of the test compound.

The FaSSIF (170 ml) preparation 100 mg of lecithin and 274 mg (anhyd equiv) of NaTaurocholate were dissolved in about 150 ml of pH 6.5 buffer. The solution was made to the volume of 170 ml with the pH 6.5 buffer.

The pH 6.5 buffer solution (1 L) preparation 4.083 g KH₂PO₄ and 7.456 g KCl were dissolved in 800 ml of water, with 100 ml 0.1 M NaOH subsequently added. The solution was made to the volume of 1 L with water. The pH value of the buffer solution was measured and adjusted to be 6.50±0.1.

Standard solutions for UPLC calibration and solubility calculation 2 μM, 20 μM and 200 μM DMSO (50% ACN water) solutions.

### UPLC Method and Parameter

Instrument: Waters ACQUITY UPLC System
Column: Waters ACQUITY UPLC BEH C18 (1.7 μm, 2.1×50 mm)
Mobile phase: A: 0.1% TFA in water/B: 0.1% TFA in CAN
Gradient: 0 min (A 95%/B 5%), 2 min (A 5%/B 95%), 2.5 min (A 5%/B 95%), 2.6 min (A 95%/B 5%), 3 min (A 95%/B 5%)
Flow rate: 0.8 mL/min; column temperature: 40° C.; injection volume: 1.0 μL; UV detection: 280 nm

d. CLND Solubility Assay

Kinetic solubility of a compound may be evaluated by the CLND (ChemiLuminescent Nitrogen Detection) solubility assay, based on known protocols (see, e.g., Bhattachar S. N.; Wesley J. A.; Seadeek C., Evaluation of the Chemiluminescent Nitrogen Detector for Solubility Determinations to Support Drug Discovery, J. Pharm. Biomed. Anal. 2006 (41):152-157; Kestranek A, Chervenek A, Logenberger J, Placko S. Chemiluminescent Nitrogen Detection (CLND) to Measure Kinetic Aqueous Solubility, Curr Protoc Chem Biol., 2013, 5(4):269-80). Typically, 5 μl of 10 mM DMSO stock solution of the test compound was diluted to 100 μl with pH7.4 phosphate buffered saline, equilibrated for 1 hour at room temperature, filtered through Millipore MultiscreenHTS-PCF filter plates (MSSL BPC). The filtrate is quantified by suitably calibrated flow injection Chemi-Luminescent Nitrogen Detection.

### 2. Assay Data

Compounds of Examples E1-E21, E25-E28, E30, E31, E34, and E37-E41 were tested in the recombinant cellular LRRK2 AlphaScreen assay and exhibited a pIC50 of ≥6.5.

Compounds of Examples E1-E9, E11, E13-E17, E25-E28, E30, E31, E34, and E37-E41 were tested in the recombinant cellular LRRK2 AlphaScreen assay and exhibited a pIC50 of ≥7.

Example 1 exhibited an pIC50 of 8.3 in the the recombinant cellular LRRK2 AlphaScreen assay. In additiona, E29, E32, E33, and E42 exhibited an pIC50 of 8.2, 8.0, 8.0 and 8.7, respectively.

Compounds of Examples E3-E6, E10-E12, E17-E21, and E25-E31 were tested in the full length G2019 human LRRK2 Inhibition Mass Spectrometry assay and exhibited a pIC50 of ≥6.5. Example 32 exhibited exhibited an pIC50 of 8.0.

3. Sequence listing

SEQ ID NO: 1 Primers used for PCR cloning of Human G2019 LRRK2 plasmids preparation: pHTBV-F
5′-GATCTCGACGGGCGCGGATCCACCATGGATTACAAGGATGACGACGAT-3′
SEQ ID NO: 2 Primers used for PCR cloning of Human G2019 LRRK2 plasmids preparation: LRRK2 wt-F1
5′-CATGGATTACAAGGATGACGACGATAAGATGGCTAGTGGCAGCTGTCAG-3′
SEQ ID NO: 3 Primers used for PCR cloning of Human G2019 LRRK2 plasmids preparation: LRRK2 wt-R1
5′-GTTCACGAGATCCACTATTCAGTAAGAGTTCCACCAATTTGGGACTG-3′
SEQ ID NO: 4 Primers used for PCR cloning of Human G2019 LRRK2 plasmids preparation: LRRK2 wt-F2
5′- GAATAGTGGATCTCGTGAACAAG-3′
SEQ ID NO: 5 Primers used for PCR cloning of Human G2019 LRRK2 plasmids preparation: LRRK2 wt-R2
5′- GTCAGACAAACTGCTTGGAACCAGC-3′
SEQ ID NO: 6 Primers used for PCR cloning of Human G2019 LRRK2 plasmids preparation: LRRK2 wt-F3
5′-CTGGTTCCAAGCAGTTTGTCTGACCACAGGCCTGTGATAG-3′
SEQ ID NO: 7 Primers used for PCR cloning of Human G2019 LRRK2 plasmids preparation: pHTBV-R
5′- GTTCTAGCCAAGCTTGGTACCCTATTACTCAACAGATGTTCGTCTC-3′
SEQ ID NO: 8 G2019 Full length Flag-LRRK2 coding sequence
atggattacaaggatgacgacgataagATGGCTAGTGGCAGCTGTCAGGGGTGCGAAGAGGACGAGGAAACTCTGAAGAAGTTGATAGTCAGGCTG
AACAATGTCCAGGAAGGAAAACAGATAGAAACGCTGGTCCAAATCCTGGAGGATCTGCTGGTGTTCACGTACTCCGAGCACGCCTCCAAGTTATTT
CAAGGCAAAAATATCCATGTGCCTCTGTTGATCGTCTTGGACTCCTATATGAGAGTCGCGAGTGTGCAGCAGGTGGGTTGGTCACTTCTGTGCAAA
TTAATAGAAGTCTGTCCAGGTACAATGCAAAGCTTAATGGGACCCCAGGATGTTGGAAATGATTGGGAAGTCCTTGGTGTTCACCAATTGATTCTT
AAAATGCTAACAGTTCATAATGCCAGTGTAAACTTGTCAGTGATTGGACTGAAGACCTTAGATCTCCTCCTAACTTCAGGTAAAATCACCTTGCTG
ATACTGGATGAAGAAAGTGATATTTTCATGTTAATTTTTGATGCCATGCACTCATTTCCAGCCAATGATGAAGTCCAGAAACTTGGATGCAAAGCT
TTACATGTGACTGTTTGGAGAGTCTCAGAGGAGCAACTGACTGAATTTGTTGAGAACAAAGATTATATGATATTGTTAAGTGCGTTAACAAATTTT
AAAGATGAAGAGGAAATTGTGCTTCATGTGCTGCATTGTTTACATTCCCTAGCGATTCCTTGCAATAATGTGGAAGTCCTCATGAGTGGCAATGTC
AGGTGTTATAATATTGTGGTGGAAGCTATGAAAGCATTCCCTATGAGTGAAAGAATTCAAGAAGTGAGTTGCTGTTTGCTCCATAGGCTTACATTA
GGTAATTTTTTCAATATCCTGGTATTAAACGAAGTCCATGAGTTTGTGGTGAAAGCTGTGCAGCAGTACCCAGAGAATGCAGCATTGCAGATCTCA
GCGCTCAGCTGTTTGGCCCTCCTCACTGAGACTATTTTCTTAAATCAAGATTTAGAGGAAAAGAATGAGAATCAAGAGAATGATGATGAGGGGGAA
GAAGATAAATTGTTTTGGCTGGAAGCCTGTTACAAAGCATTAACGTGGCATAGAAAGAACAAGCACGTGCAGGAGGCCGCATGCTGGGCACTAAAT
AATCTCCTTATGTACCAAAACAGTTTACATGAGAAGATTGGAGATGAAGATGGCCATTTCCCAGCTCATAGGGAAGTGATGCTCTCCATGCTGATG
CATTCTTCATCAAAGGAAGTTTTCCAGGCATCTGCGAATGCATTGTCAACTCTCTTAGAACAAAATGTTAATTTCAGAAAAATACTGTTATCAAAA
GGAATACACCTGAATGTTTTGGGAGTTAATGCAAAGCATATACATTCTCCTGAAGTGGCTGAAAGTGGCTGTAAAATGCTAAATCATCTTTTTGAA
GGAAGCAACACTTCCCTGGATATAATGGCAGCAGTGGTCCCCAAAATACTAACAGTTATGAAACGTCATGAGACATCATTACCAGTGCAGCTGGAG
GCGCTTCGAGCTATTTTACATTTTATAGTGCCTGGCATGCCAGAAGAATCCAGGGAGGATACAGAATTTCATCATAAGCTAAATATGGTTAAAAAA
CAGTGTTTCAAGAATGATATTCACAAACTGGTCCTAGCAGCTTTGAACAGGTTCATTGGAAATCCTGGGATTCAGAAATGTGGATTAAAAGTAATT
TCTTCTATTGTACATTTTCCTGATGCATTAGAGATGTTATCCCTGGAAGGTGCTATGGATTCAGTGCTTCACACACTGCAGATGTATCCAGATGAC
CAAGAAATTCAGTGTCTGGGTTTAAGTCTTATAGGATACTTGATTACAAAGAAGAATGTGTTCATAGGAACTGGACATCTGCTGGCAAAAATTCTG
GTTTCCAGCTTATACCGATTTAAGGATGTTGCTGAAATACAGACTAAAGGATTTCAGACAATCTTAGCAATCCTCAAATTGTCAGCATCTTTTTCT
AAGCTGCTGGTGCATCATTCATTTGACTTAGTAATATTCCATCAAATGTCTTCCAATATCATGGAACAAAAGGATCAACAGTTTCTAAACCTCTGT
TGCAAGTGTTTTGCAAAAGTAGCTATGGATGATTACTTAAAAAATGTGATGCTAGAGAGAGCGTGTGATCAGAATAACAGCATCATGGTTGAATGC
TTGCTTCTATTGGGAGCAGATGCCAATCAAGCAAAGGAGGGATCTTCTTTAATTTGTCAGGTATGTGAGAAAGAGAGCAGTCCCAAATTGGTGGAA
CTCTTACTGAATAGTGGATCTCGTGAACAAGATGTACGAAAAGCGTTGACGATAAGCATTGGGAAAGGTGACAGCCAGATCATCAGCTTGCTCTTA
AGGAGGCTGGCCCTGGATGTGGCCAACAATAGCATTTGCCTTGGAGGATTTTGTATAGGAAAAGTTGAACCTTCTTGGCTTGGTCCTTTATTTCCA
GATAAGACTTCTAATTTAAGGAAACAAACAAATATAGCATCTACACTAGCAAGAATGGTGATCAGATATCAGATGAAAAGTGCTGTGGAAGAAGGA
ACAGCCTCAGGCAGCGATGGAAATTTTTCTGAAGATGTGCTGTCTAAATTTGATGAATGGACCTTTATTCCTGACTCTTCTATGGACAGTGTGTTT
GCTCAAAGTGATGACCTGGATAGTGAAGGAAGTGAAGGCTCATTTCTTGTGAAAAAGAAATCTAATTCAATTAGTGTAGGAGAATTTTACCGAGAT
GCCGTATTACAGCGTTGCTCACCAAATTTGCAAAGACATTCCAATTCCTTGGGGCCCATTTTTGATCATGAAGATTTACTGAAGCGAAAAAGAAAA
ATATTATCTTCAGATGATTCACTCAGGTCATCAAAACTTCAATCCCATATGAGGCATTCAGACAGCATTTCTTCTCTGGCTTCTGAGAGAGAATAT
ATTACATCACTAGACCTTTCAGCAAATGAACTAAGAGATATTGATGCCCTAAGCCAGAAATGCTGTATAAGTGTTCATTTGGAGCATCTTGAAAAG
CTGGAGCTTCACCAGAATGCACTCACGAGCTTTCCACAACAGCTATGTGAAACTCTGAAGAGTTTGACACATTTGGACTTGCACAGTAATAAATTT
ACATCATTTCCTTCTTATTTGTTGAAAATGAGTTGTATTGCTAATCTTGATGTCTCTCGAAATGACATTGGACCCTCAGTGGTTTTAGATCCTACA
GTGAAATGTCCAACTCTGAAACAGTTTAACCTGTCATATAACCAGCTGTCTTTTGTACCTGAGAACCTCACTGATGTGGTAGAGAAACTGGAGCAG
CTCATTTTAGAAGGAAATAAAATATCAGGGATATGCTCCCCCTTGAGACTGAAGGAACTGAAGATTTTAAACCTTAGTAAGAACCACATTTCATCC
CTATCAGAGAACTTTCTTGAGGCTTGTCCTAAAGTGGAGAGTTTCAGTGCCAGAATGAATTTTCTTGCTGCTATGCCTTTCTTGCCTCCTTCTATG
ACAATCCTAAAATTATCTCAGAACAAATTTTCCTGTATTCCAGAAGCAATTTTAAATCTTCCACACTTGCGGTCTTTAGATATGAGCAGCAATGAT
ATTCAGTACCTACCAGGTCCCGCACACTGGAAATCTTTGAACTTAAGGGAACTCTTATTTAGCCATAATCAGATCAGCATCTTGGACTTGAGTGAA
AAAGCATATTTATGGTCTAGAGTAGAGAAACTGCATCTTTCTCACAATAAACTGAAAGAGATTCCTCCTGAGATTGGCTGTCTTGAAAATCTGACA
TCTCTGGATGTCAGTTACAACTTGGAACTAAGATCCTTTCCCAATGAAATGGGGAAATTAAGCAAAATATGGGATCTTCCTTTGGATGAACTGCAT
CTTAACTTTGATTTTAAACATATAGGATGTAAAGCCAAAGACATCATAAGGTTTCTTCAACAGCGATTAAAAAAGGCTGTGCCTTATAACCGAATG
AAACTTATGATTGTGGGAAATACTGGGAGTGGTAAAACCACCTTATTGCAGCAATTAATGAAAACCAAGAAATCAGATCTTGGAATGCAAAGTGCC
ACAGTTGGCATAGATGTGAAAGACTGGCCTATCCAAATAAGAGACAAAAGAAAGAGAGATCTCGTCCTAAATGTGTGGGATTTTGCAGGTCGTGAG
GAATTCTATAGTACTCATCCCCATTTTATGACGCAGCGAGCATTGTACCTTGCTGTCTATGACCTCAGCAAGGGACAGGCTGAAGTTGATGCCATG
AAGCCTTGGCTCTTCAATATAAAGGCTCGCGCTTCTTCTTCCCCTGTGATTCTCGTTGGCACACATTTGGATGTTTCTGATGAGAAGCAACGCAAA
GCCTGCATGAGTAAAATCACCAAGGAACTCCTGAATAAGCGAGGGTTCCCTGCCATACGAGATTACCACTTTGTGAATGCCACCGAGGAATCTGAT
GCTTTGGCAAAACTTCGGAAAACCATCATAAACGAGAGCCTTAATTTCAAGATCCGAGATCAGCTTGTTGTTGGACAGCTGATTCCAGACTGCTAT
GTAGAACTTGAAAAAATCATTTTATCGGAGCGTAAAAATGTGCCAATTGAATTTCCCGTAATTGACCGGAAACGATTATTACAACTAGTGAGAGAA
AATCAGCTGCAGTTAGATGAAAATGAGCTTCCTCACGCAGTTCACTTTCTAAATGAATCAGGAGTCCTTCTTCATTTTCAAGACCCAGCACTGCAG
TTAAGTGACTTGTACTTTGTGGAACCCAAGTGGCTTTGTAAAATCATGGCACAGATTTTGACAGTGAAAGTGGAAGGTTGTCCAAAACACCCTAAG
GGAATTATTTCGCGTAGAGATGTGGAAAAATTTCTTTCAAAGAAAAGGAAATTTCCAAAGAACTACATGTCACAGTATTTTAAGCTCCTAGAAAAA
TTCCAGATTGCTTTGCCAATAGGAGAAGAATATTTGCTGGTTCCAAGCAGTTTGTCTGACCACAGGCCTGTGATAGAGCTTCCCCATTGTGAGAAC
TCTGAAATTATCATCCGACTATATGAAATGCCTTATTTTCCAATGGGATTTTGGTCAAGATTAATCAATCGATTACTTGAGATTTCACCTTACATG
CTTTCAGGGAGAGAACGAGCACTTCGCCCAAACAGAATGTATTGGCGACAAGGCATTTACTTAAATTGGTCTCCTGAAGCTTATTGTCTGGTAGGA
TCTGAAGTCTTAGACAATCATCCAGAGAGTTTCTTAAAAATTACAGTTCCTTCTTGTAGAAAAGGCTGTATTCTTTTGGGCCAAGTTGTGGACCAC
ATTGATTCTCTCATGGAAGAATGGTTTCCTGGGTTGCTGGAGATTGATATTTGTGGTGAAGGAGAAACTCTGTTGAAGAAATGGGCATTATATAGT
TTTAATGATGGTGAAGAACATCAAAAAATCTTACTTGATGACTTGATGAAGAAAGCAGAGGAAGGAGATCTCTTAGTAAATCCAGATCAACCAAGG
CTCACCATTCCAATATCTCAGATTGCCCCTGACTTGATTTTGGCTGACCTGCCTAGAAATATTATGTTGAATAATGATGAGTTGGAATTTGAACAA
GCTCCAGAGTTTCTCCTAGGTGATGGCAGTTTTGGATCAGTTTACCGAGCAGCCTATGAAGGAGAAGAAGTGGCTGTGAAGATTTTTAATAAACAT
ACATCACTCAGGCTGTTAAGACAAGAGCTTGTGGTGCTTTGCCACCTCCACCACCCCAGTTTGATATCTTTGCTGGCAGCTGGGATTCGTCCCCGG
ATGTTGGTGATGGAGTTAGCCTCCAAGGGTTCCTTGGATCGCCTGCTTCAGCAGGACAAAGCCAGCCTCACTAGAACCCTACAGCACAGGATTGCA
CTCCACGTAGCTGATGGTTTGAGATACCTCCACTCAGCCATGATTATATACCGAGACCTGAAACCCCACAATGTGCTGCTTTTCACACTGTATCCC
AATGCTGCCATCATTGCAAAGATTGCTGACTACGGCATTGCTCAGTACTGCTGTAGAATGGGGATAAAAACATCAGAGGGCACACCAGGGTTTCGT
GCACCTGAAGTTGCCAGAGGAAATGTCATTTATAACCAACAGGCTGATGTTTATTCATTTGGTTTACTACTCTATGACATTTTGACAACTGGAGGT
AGAATAGTAGAGGGTTTGAAGTTTCCAAATGAGTTTGATGAATTAGAAATACAAGGAAAATTACCTGATCCAGTTAAAGAATATGGTTGTGCCCCA
TGGCCTATGGTTGAGAAATTAATTAAACAGTGTTTGAAAGAAAATCCTCAAGAAAGGCCTACTTCTGCCCAGGTCTTTGACATTTTGAATTCAGCT
GAATTAGTCTGTCTGACGAGACGCATTTTATTACCTAAAAACGTAATTGTTGAATGCATGGTTGCTACACATCACAACAGCAGGAATGCAAGCATT
TGGCTGGGCTGTGGGCACACCGACAGAGGACAGCTCTCATTTCTTGACTTAAATACTGAAGGATACACTTCTGAGGAAGTTGCTGATAGTAGAATA
TTGTGCTTAGCCTTGGTGCATCTTCCTGTTGAAAAGGAAAGCTGGATTGTGTCTGGGACACAGTCTGGTACTCTCCTGGTCATCAATACCGAAGAT
GGGAAAAAGAGACATACCCTAGAAAAGATGACTGATTCTGTCACTTGTTTGTATTGCAATTCCTTTTCCAAGCAAAGCAAACAAAAAAATTTTCTT
TTGGTTGGAACCGCTGATGGCAAGTTAGCAATTTTTGAAGATAAGACTGTTAAGCTTAAAGGAGCTGCTCCTTTGAAGATACTAAATATAGGAAAT
GTCAGTACTCCATTGATGTGTTTGAGTGAATCCACAAATTCAACGGAAAGAAATGTAATGTGGGGAGGATGTGGCACAAAGATTTTCTCCTTTTCT
AATGATTTCACCATTCAGAAACTCATTGAGACAAGAACAAGCCAACTGTTTTCTTATGCAGCTTTCAGTGATTCCAACATCATAACAGTGGTGGTA
GACACTGCTCTCTATATTGCTAAGCAAAATAGCCCTGTTGTGGAAGTGTGGGATAAGAAAACTGAAAAACTCTGTGGACTAATAGACTGCGTGCAC
TTTTTAAGGGAGGTAATGGTAAAAGAAAACAAGGAATCAAAACACAAAATGTCTTATTCTGGGAGAGTGAAAACCCTCTGCCTTCAGAAGAACACT
GCTCTTTGGATAGGAACTGGAGGAGGCCATATTTTACTCCTGGATCTTTCAACTCGTCGACTTATACGTGTAATTTACAACTTTTGTAATTCGGTC
AGAGTCATGATGACAGCACAGCTAGGAAGCCTTAAAAATGTCATGCTGGTATTGGGCTACAACCGGAAAAATACTGAAGGTACACAAAAGCAGAAA
GAGATACAATCTTGCTTGACCGTTTGGGACATCAATCTTCCACATGAAGTGCAAAATTTAGAAAAACACATTGAAGTGAGAAAAGAATTAGCTGAA
AAAATGAGACGAACATCTGTTGAGTAA
SEQ ID NO: 9 Translated protein sequence for human G2019 Full length LRRK2 flag tagged protein
MDYKDDDDKMASGSCQGCEEDEETLKKLIVRLNNVQEGKQIETLVQILEDLLVFTYSEHASKLFQGKNIHVPLLIVLDSYMRVASVQQVGWSLLCK
LIEVCPGTMQSLMGPQDVGNDWEVLGVHQLILKMLTVHNASVNLSVIGLKTLDLLLTSGKITLLILDEESDIFMLIFDAMHSFPANDEVQKLGCKA
LHVLFERVSEEQLTEFVENKDYMILLSALTNFKDEEEIVLHVLHCLHSLAIPCNNVEVLMSGNVRCYNIVVEAMKAFPMSERIQEVSCCLLHRLTL
GNFFNILVLNEVHEFVVKAVQQYPENAALQISALSCLALLTETIFLNQDLEEKNENQENDDEGEEDKLFWLEACYKALTWHRKNKHVQEAACWALN
NLLMYQNSLHEKIGDEDGHFPAHREVMLSMLMHSSSKEVFQASANALSTLLEQNVNFRKILLSKGIHLNVLELMQKHIHSPEVAESGCKMLNHLFE
GSNTSLDIMAAVVPKILTVMKRHETSLPVQLEALRAILHFIVPGMPEESREDTEFHHKLNMVKKQCFKNDIHKLVLAALNRFIGNPGIQKCGLKVI
SSIVHFPDALEMLSLEGAMDSVLHTLQMYPDDQEIQCLGLSLIGYLITKKNVFIGTGHLLAKILVSSLYRFKDVAEIQTKGFQTILAILKLSASFS
KLLVHHSFDLVIFHQMSSNIMEQKDQQFLNLCCKCFAKVAMDDYLKNVMLERACDQNNSIMVECLLLLGADANQAKEGSSLICQVCEKESSPKLVE
LLLNSGSREQDVRKALTISIGKGDSQIISLLLRRLALDVANNSICLGGFCIGKVEPSWLGPLFPDKTSNLRKQTNIASTLARMVIRYQMKSAVEEG
TASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRK
ILSSDDSLRSSKLQSHMRHSDSISSLASEREYITSLDLSANELRDIDALSQKCCISVHLEHLEKLELHQNALTSFPQQLCETLKSLTHLDLHSNKF
TSFPSYLLKMSCIANLDVSRNDIGPSVVLDPTVKCPTLKQFNLSYNQLSFVPENLTDVVEKLEQLILEGNKISGICSPLRLKELKILNLSKNHISS
LSENFLEACPKVESFSARMNFLAAMPFLPPSMTILKLSQNKFSCIPEAILNLPHLRSLDMSSNDIQYLPGPAHWKSLNLRELLFSHNQISILDLSE
KAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRM
KLMIVGNIGSGKTILLQQLMKTKKSDLGMQSATVGIDVKDWPIQIRDKRKRDLVLNVVVDFAGREEFYSTHPHFMTQRALYLAVYDLSKGQAEVDA
MKPWLFNIKARASSSPVILVGTHLDVSDEKQRKACMSKITKELLNKRGFPAIRDYHFVNATEESDALAKLRKTIINESLNFKIRDQLVVGQLIPDC
YVELEKIILSERKNVPIEFPVIDRKRLLQLVRENQLQLDENELPHAVHFLNESGVLLHFQDPALQLSDLYFVEPKWLCKIMAQILTVKVEGCPKHP
KGIISRRDVEKFLSKKRKFPKNYMSQYFKLLEKFQIALPIGEEYLLVPSSLSDHRPVIELPHCENSEIIIRLYEMPYFPMGFWSRLINRLLEISPY
MLSGRERALRPNRMYWRQGIYLNWSPEAYCLVGSEVLDNHPESFLKITVPSCRKGCILLGQVVDHIDSLMEEWFPGLLEIDICGEGETLLKKWALY
SFNDGEEHQKILLDDLMKKAEEGDLLVNPDQPRLTIPISQIAPDLILADLPRNIMLNNDELEFEQAPEFLLGDGSFGSVYRAAYEGEEVAVKIFNK
HTSLRLLRQELVVLCHLHHPSLISLLAAGIRPRMLVMELASKGSLDRLLQQDKASLTRTLQHRIALHVADGLRYLHSAMIIYRDLKPHNVLLFTLY
PNAAIIAKIADYGIAQYCCRMGIKTSEGTPGFRAPEVARGNVIYNQQADVYSFGLLLYDILTTGGRIVEGLKFPNEFDELEIQGKLPDPVKEYGCA
PWPMVEKLIKQCLKENPQERPTSAQVFDILNSAELVCLTRRILLPKNVIVECMVATHHNSRNASIWLGCGHTDRGQLSFLDLNTEGYTSEEVADSR
ILCLALVHLPVEKESWIVSGTQSGTLLVINTEDGKKRHTLEKMTDSVTCLYCNSFSKQSKQKNFLLVGTADGKLAIFEDKTVKLKGAAPLKILNIG
NVSTPLMCLSESTNSTERNVMWGGCGTKIFSFSNDFTIQKLIETRTSQLFSYAAFSDSNIITVVVDTALYIAKQNSPVVEVWDKKTEKLCGLIDCV
HFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQ
KEIQSCLTVWDINLPHEVQNLEKHIEVRKELAEKMRRTSVE
SEQ ID NO: 10: ‘LRRKtide’ peptide
H-RLGRDKYKTLRQIRQ-OH

1-20. (canceled)

21. A compound of Formula (I): 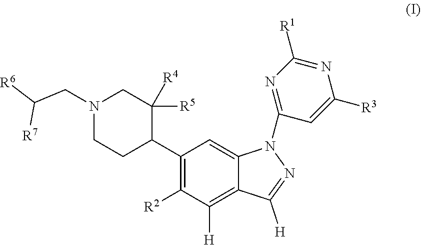
wherein
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃ haloalkyl;
R³ is selected from the group consisting of:
a) an N-linked 4-6 membered heterocyclyl ring optionally substituted with one or two substituents independently selected from the group consisting of halo, hydroxyl, C₁₋₆alkyl and C₁₋₆ alkoxyl,
wherein:
the C₁₋₆alkyl group is optionally substituted with one or two substituents independently selected from the group consisting of: halo, hydroxyl, C₁₋₃alkoxy and cyclopropyl, and
the C₁₋₆ alkoxyl group is optionally substituted with one or two substitutents independently selected from the group consisting of halo, hydroxyl and C₁₋₃ alkoxyl; and
when the N-linked 4-6 membered heterocyclyl ring contains a substitutable nitrogen atom, the N-linked 4-6 membered heterocyclyl ring optionally is substituted by a 4-6 membered heterocyclyl ring;
wherein:
the 4-6 membered heterocyclyl ring optionally is substituted with one, two or three substitutents independently selected from halo, hydroxyl, and C₁₋₃ alkoxyl,
provided that:
the 4-6 membered heterocyclyl ring is attached to said substitutable nitrogen atom of the N-linked 4-6 membered heterocyclyl ring;
b) NHR⁸; and
c) OR⁸;
R⁴ and R⁵ are independently selected from the group consisting of H, hydroxyl and halo;
R⁶ is halo, hydroxyl or —(CH₂)ₙSO₂C₁₋₃alkyl;
wherein:
n is 0, 1, 2 or 3;
R⁷ is selected from the group consisting of H, Cyclopropyl, C₁₋₃ alkyl, —CH₂CH₂— and CH₂CH₂CH₂—;
wherein:
the C₁₋₃alkyl optionally is substituted with one, two or three substitutents independently selected from the group consisting of halo, hydroxyl, and C₁₋₃ alkoxyl;
in the —CH₂CH₂CH₂—, one terminal carbon joins with the carbon atom to which another terminal carbon atom is attached to form a ring;
R⁸ is independently selected from the group consisting of a C₄₋₆ cycloalkyl and a 4-6 membered heterocyclyl that contains nitrogen or oxygen
wherein:
the C₄₋₆ cycloalkyl group optionally substituted with one, two or three substituents independently selected from the group consisting of halo, hydroxyl, C₁₋₃ alkoxyl and C₁₋₃ alkyl;
the 4-6 membered heterocyclyl that contains nitrogen or oxygen optionally is substituted with one or more substitutents independently selected from the group consisting of halo, hydroxyl, C₁₋₃ alkoxyl and C₁₋₃ alkyl,
wherein:
the C₁₋₃ alkyl group of the C₄₋₆ cycloalkyl group and the 4-6 membered heterocyclyl group, respectively, optionally is substituted with one two or three substituents independently selected from halo and hydroxyl; or
a pharmaceutically acceptable salt thereof.

22. The compound of Formula (I) or pharmaceutically acceptable salt according to claim 21, wherein R¹ is selected from the group consisting of C₁₋₃ alkyl and C₁₋₃ alkoxyl.

23. The compound of Formula (I) or pharmaceutically acceptable salt according to claim 21, wherein R² is selected from the group consisting of H, halo and C₁₋₃alkyl.

24. The compound of Formula (I) or pharmaceutically acceptable salt according to claim 21, wherein R⁴ and R⁵ are independently selected from the group consisting of H and fluoro.

25. The compound of Formula (I) or pharmaceutically acceptable salt according to claim 24, wherein R⁴ and R⁵ are both H.

26. The compound of Formula (I) or pharmaceutically acceptable salt according to claim 21, wherein:
R³ is an N-linked 4-6 membered heterocyclyl ring optionally substituted with one or two substituents independently selected from the group consisting of halo, hydroxyl, C₁₋₃alkyl and C₁₋₃ alkoxyl;
wherein:
the C₁₋₃ alkyl group optionally is substituted with one or two substituents independently selected from the group consisting of halo, hydroxyl and C₁₋₃alkoxy, and
the C₁₋₃ alkoxyl group is optionally substituted with one or two substitutents independently selected from halo, hydroxyl and C₁₋₃ alkoxyl.

27. The compound of Formula (I) or pharmaceutically acceptable salt according to claim 21, wherein R⁶ is fluoro or hydroxyl.

28. The compound of Formula (I) or pharmaceutically acceptable salt according to claim 21, wherein R⁷ is H.

29. A compound which is 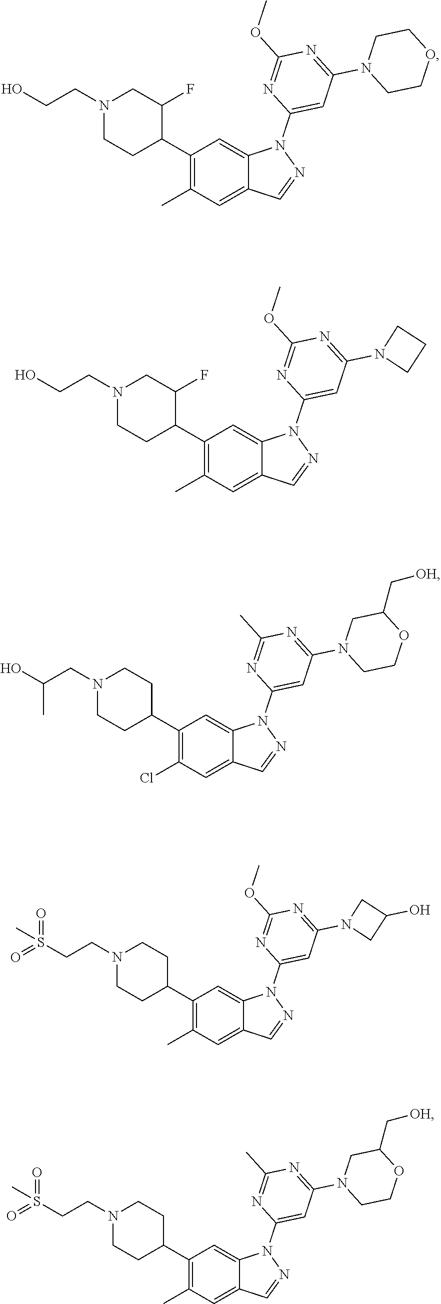 [SMILES: COc1nc(N2CCOCC2)cc(-n2ncc3cc(C)c(C4CCN(CCO)CC4F)cc32)n1 ; COc1nc(N2CCC2)cc(-n2ncc3cc(C)c(C4CCN(CCO)CC4F)cc32)n1 ; Cc1nc(N2CCOC(CO)C2)cc(-n2ncc3cc(Cl)c(C4CCN(CC(C)O)CC4)cc32)n1 ; COc1nc(N2CC(O)C2)cc(-n2ncc3cc(C)c(C4CCN(CCS(C)(=O)=O)CC4)cc32)n1 ; Cc1nc(N2CCOC(CO)C2)cc(-n2ncc3cc(C)c(C4CCN(CCS(C)(=O)=O)CC4)cc32)n1] 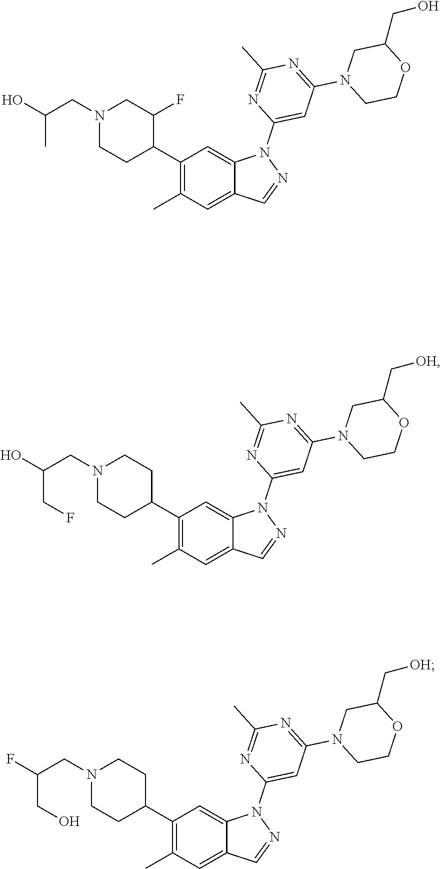 [SMILES: Cc1nc(N2CCOC(CO)C2)cc(-n2ncc3cc(C)c(C4CCN(CC(C)O)CC4F)cc32)n1 ; Cc1nc(N2CCOC(CO)C2)cc(-n2ncc3cc(C)c(C4CCN(CC(O)CF)CC4)cc32)n1 ; Cc1nc(N2CCOC(CO)C2)cc(-n2ncc3cc(C)c(C4CCN(CC(F)CO)CC4)cc32)n1]
or a pharmaceutically acceptable salt thereof.

30. A pharmaceutical composition comprising a compound of Formula (I) or a pharmaceutically acceptable salt thereof according to claim 21 and at least one pharmaceutically acceptable excipient(s).

31. A method for treating a neurodegenerative disease, which comprises administering to a subject in need thereof a therapeutically effective amount of a compound of Formula (I) or a pharmaceutically acceptable salt according to claim 21.

32. The method for treating a neurodegenerative disease according to claim 31, wherein the neurodegenerative disease is Parkinson's disease.

33. The method for treating a neurodegenerative disease according to claim 32, wherein the subject is a human.

34. The method for treating a neurodegenerative disease according to claim 33, wherein the subject is a human expressing the G2019S mutation in the LRRK2 kinase.


SEQUENCE LISTING (10 sequences)

SEQ ID NO: 1 - DNA, 48 bp, Artificial
  Primers used for PCR cloning of Human G2019 LRRK2 plasmids preparation pHTBV-F
gatctcgacgggcgcggatccaccatggattacaaggatgacgacgat

SEQ ID NO: 2 - DNA, 49 bp, Artificial
  Primers used for PCR cloning of Human G2019 LRRK2 plasmids preparation LRRK2 wt-F1
catggattacaaggatgacgacgataagatggctagtggcagctgtcag

SEQ ID NO: 3 - DNA, 47 bp, Artificial
  Primers used for PCR cloning of Human G2019 LRRK2 plasmids preparation LRRK2 wt-R1
gttcacgagatccactattcagtaagagttccaccaatttgggactg

SEQ ID NO: 4 - DNA, 23 bp, Artificial
  Primers used for PCR cloning of Human G2019 LRRK2 plasmids preparation LRRK2 wt-F2
gaatagtggatctcgtgaacaag

SEQ ID NO: 5 - DNA, 25 bp, Artificial
  Primers used for PCR cloning of Human G2019 LRRK2 plasmids preparation LRRK2 wt-R2
gtcagacaaactgcttggaaccagc

SEQ ID NO: 6 - DNA, 40 bp, Artificial
  Primers used for PCR cloning of Human G2019 LRRK2 plasmids preparation LRRK2 wt-F3
ctggttccaagcagtttgtctgaccacaggcctgtgatag

SEQ ID NO: 7 - DNA, 46 bp, Artificial
  Primers used for PCR cloning of Human G2019 LRRK2 plasmids preparation pHTBV-R
gttctagccaagcttggtaccctattactcaacagatgttcgtctc

SEQ ID NO: 8 - DNA, 7611 bp, Artificial
  G2019  Full length Flag-LRRK2 coding sequence
atggattacaaggatgacgacgataagatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcacgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatactggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggaattatttcgcgtagagatgtggaaaaatttctttcaaagaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaa

SEQ ID NO: 9 - protein, 2536 aa, Artificial
  Translated protein sequence for human G2019 Full length LRRK2 flag tagged protein
MDYKDDDDKMASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 10 - protein, 15 aa, Artificial
  ′LRRKtide′ peptide
RLGRDKYKTLRQIRQ